(19) United States
(12) Patent Application Publication
(10) Pub. No.: US 2025/0298013 A1
(43) Pub. Date: Sep. 25, 2025

(54) SURFACE ACTIVATION OF MATERIALS AND MICROARRAY PRINTING FOR USE IN BIOLOGICAL ANALYSIS

(71) Applicant: Luna Labs USA, LLC, Charlottesville, VA (US)

(72) Inventors: Judy M. OBLIOSCA, Charlottesville, VA (US); Dimpal PATEL, Charlottesville, VA (US); Zhiguo ZHOU, Charlottesville, VA (US); Yang XU, Charlottesville, VA (US); Christopher K. TISON, Charlottesville, VA (US); Kate JOHNSON, Charlottesville, VA (US); Tammy FERGUSON, Charlottesville, VA (US); Victoria STAGNARO, Charlottesville, VA (US)

(21) Appl. No.: 18/862,398

(22) PCT Filed: Mar. 3, 2023
(86) PCT No.: PCT/US2023/014485

§ 371 Date: Nov. 1, 2024

Related U.S. Application Data
(60) Provisional application No. 63/337,698, filed on May 3, 2022.

(51) Int. Cl. G01N 33/543; G01N 33/531; G01N 33/552; G01N 33/553; G01N 33/68

(52) U.S. Cl. CPC G01N 33/54366; G01N 33/531; G01N 33/54393; G01N 33/552; G01N 33/553; G01N 33/68

(57) ABSTRACT

Provided herein are methods of surface functionalization, array construction, and/or blocking as well as substrates that are surface functionalized and blocked from non-specific bindings. Also provided herein are blocking solutions, blocking agents, and kits comprising the same. Further provided are methods of using functionalized and blocked surfaces for analyzing samples, such as in an SPR analysis.

### GOVERNMENT'S INTERESTS

The invention was made with government support under the following contract numbers: Contract No. 140D6318C0012 awarded by DARPA, Contract No. HHSN272201800026C awarded by NIH, Contract Nos. W81XWH-20-P-0023, W81XWH22C0024, and W81XWH-14-C-0146 awarded by USAMRAA, and Contract Nos. 75D30119P06302 and 75D30120C09984 awarded by CDC. The U.S. government has certain rights in the invention.

### BACKGROUND

### Field of the Disclosure

In various embodiments, the present disclosure generally relates to surface functionalization, array constructions, and/or blocking, and uses thereof, such as for surface plasmon resonance (SPR) analysis.

### Background

In the analysis of biomarkers present in a wide variety of biofluids (e.g., serum, plasma, whole blood, urine, cerebrospinal fluid, etc.), prevention of non-specific binding of biomolecules is a challenge. Biofluids contain unwanted proteins that can adsorb onto the assay's solid substrate or membrane and form non-specific interactions with biomolecules on the surface, resulting in high background signal and poor detection of target markers present in low concentrations. To produce highly sensitive and specific biomarker assays, surface functionalization of materials, construction of sensing probes, and blocking are essential to specifically capture the biomolecules of interest, prevent interfering biomolecules from interacting with the desired binding sites, and accurately detect target biomarkers or indicators in biofluids (1-4).

### BRIEF SUMMARY

The present disclosure is based in part on Applicant's discovery of a surface activation system protocol (termed SAS) designed to prevent non-specific binding of biomolecules during surface plasmon resonance (SPR)-based assays. However, this protocol isn't limited in applicability to SPR sensing chip surfaces, but can be extended for use on any surfaces such as metals, glass, ceramics and polymers. In embodiments herein, SPR imaging (SPRi) is used as a representative method of use of the present disclosure.

As an advanced optics-based quantitative detection method, SPR sensing has been extended to the SPRi technology for high-throughput probing of biomolecular interactions. SPRi stands out as a powerful detection tool as it rapidly detects biomarkers without the need of fluorophores and enzymes, can be used with very small samples (40 μL), and measures binding kinetics in real time and in the microarray format (5-7). The overall performance of SPRi-based assays is highly dependent on the quality of the surface functionalization and proper anchoring of the biorecognition probes onto the surfaces. In order to detect target markers at high selectivity and sensitivity, improved functionalization, printing (or spotting), and blocking strategies on the chip surface are highly desired. However, such a demand has not been properly addressed by the diagnostic field.

As discussed herein, in the absence of an efficiently activated (i.e., functionalized, printed, and/or blocked) sensing surface, non-specific binding of proteins is too high and interferes with the detection of trace amounts of target biomarkers, making it difficult to assess the status of the biomarkers and ultimately the corresponding diseases. In various embodiments, the present disclosure provides novel surface functionalization, printing, and/or blocking which can effectively eliminate unwanted non-specific binding of proteins and obtain workable detection signal, for example, in a SPR analysis.

As discussed in details below, embodiments of the present disclosure generally relate to (1) surface functionalization of a surface using a surface agent (e.g., thiolated protein A), (2) construction of sensing arrays (simultaneous printing of capture probes such as antibodies, antigens, and aptamers), and/or (3) surface treatment using blocking agents.

In a typical embodiment, the surface functionalization of (1) can include functionalizing an inert metal surface (e.g., a gold surface) with a bifunctional surface agent (e.g., a thiolated protein A solution) to provide a surface-agent-functionalized surface. The surface agent typically is bifunctional, which can be immobilized on the surface, e.g., through a thiol group, and upon immobilization, can bind to a capture molecule of interest, such as to a capture antibody through the Fc region.

For example, in some embodiments, the entire inert metal surface (such as a gold chip surface) is coated with the surface agent (e.g., a thiolated protein A solution). In some embodiments, the inert metal surface is treated with the surface agent in the presence of a press load (of appropriate force and resultant stress). In some embodiments, the inert metal surface can also be treated with the surface agent without using a press load.

In some embodiments, the surface functionalization can also be at specified locations, in other words, only certain areas of the inert metal surface are treated with the surface agent. For example, in some embodiments, the surface agent can be printed or spotted in a particular location on the inert metal surface by using a fully automated printer, such as a continuous flow microspotter (CFM). Continuous flow microspotters suitable for embodiments of the present application are not particularly limited, which include but are not limited to those exemplified herein. Surface functionalization with the surface agent printed or spotted on a surface can be particularly useful in certain cases, such as for microarray constructions.

Various surface agents are suitable for the surface functionalization. In some embodiments, the surface agent can be a protein, such as protein A, protein G, protein L, avidin, or streptavidin, or a fragment or functional variant thereof. In some embodiments, the surface agent is modified such that it can be covalently bonded to the inert surface. In some embodiments, the modified surface agent is a protein such as protein A, protein G, protein L, avidin, or streptavidin, or a fragment or functional variant thereof. In further embodiments, the modified surface agent is a protein modified with amine, carboxyl, hydroxyl, and/or thiol, which can bind to the inert metal surface through direct binding or through other types of modifications (such as carbodiimide-based reaction, siloxane network formation, etc.). In some embodiments, the surface agent can be protein A, which is modified such that protein A can be covalently bonded to the inert surface. For example, the protein A, can be modified with amine, carboxyl, hydroxyl, and/or thiol, which can bind to the inert metal surface through direct binding or through other types of modifications (such as carbodiimide-based reaction, siloxane network formation, etc.). In some specific embodiments, the surface agent is a thiolated protein A. i.e., the protein A is modified with a thiol moiety.

The inert metal surface, including inert metal film or layer, is not particularly limited. In some specific embodiments, the inert metal surface is a gold, silver, or gold/silver alloy film, preferably gold film, which can be coated on various substrates, such as metals, glass, nitrocellulose filters, ceramics, or polymers.

Typically, subsequent to the surface functionalization, construction of sensing arrays (2) is implemented. For example, in some embodiments, the sensing arrays can be carried out by simultaneously spotting of multiple arrays (e.g., capture antibodies, antigens, and/or capture aptamers) on the functionalized chip surface using a MS (multiple spotting) technique known in the art (e.g., the Luna Labs MultiSpot technology) with the aid of a CFM, for example, using a CFM available through HORIBA Scientific as described in the Examples section herein. As used herein, the term tMS” or “multiple spotting” refers to a simultaneous printing/stacking of reagents on specific spot locations of the sensing chip to effectively capture target biomarkers and indicators. Exemplified MS methods are described herein. e.g., in the Examples section. In some embodiments, the capture molecule can be surface-agent dependent, i.e., the capture molecule can bind to the inert metal surface through specific binding directly or indirectly to the surface agent. For example, a capture antibody that binds to the inert metal surface through specific binding to Protein A can be characterized as a surface-agent-dependent capture molecule. In some embodiments, the capture molecule can be surface-agent-independent, i.e., the capture molecule is capable of binding to the inert metal surface without binding to the surface agent directly or indirectly. In further embodiments, the capture molecule can be an antibody modified with amine, carboxyl, hydroxyl, and/or thiol. The sensing arrays that can be implemented are not particularly limited, which can be extended for optimal detection of a wide variety of pathology- and disease-associated biomarkers/biomacromolecules.

Typically, in surface blocking (3), blocking solutions/agents are used to reduce or prevent or eliminate (>95%) non-specific binding events, e.g., those from proteins in different sample matrices (e.g., clinical samples, animal samples, supernatants), to achieve a low background and high biomarker signal detection.

The present disclosure provides the following numbered exemplary embodiments 1-102:

Embodiment 1. A method of preparing a substrate having an inert metal surface, comprising:
a) functionalizing the inert metal surface with a surface agent to produce a surface-agent-functionalized surface; and
b) blocking the inert metal surface to reduce or prevent non-specific binding.

Embodiment 2. The method of Embodiment 1, wherein the inert metal surface is a gold, silver, or gold/silver alloy surface coated on the substrate, preferably, a gold surface.

Embodiment 3. The method of Embodiment 1 or 2, wherein the surface agent is a protein, preferably, protein A, protein G, protein L, avidin, or streptavidin.

Embodiment 4. The method of Embodiment 1 or 2, wherein the surface agent is protein A modified with amine, carboxyl, hydroxyl, and/or thiol.

Embodiment 5. The method of Embodiment 1 or 2, wherein the surface agent is a thiolated protein A, wherein the protein A is modified with a thiol.

Embodiment 6. The method of any of Embodiments 1-5, wherein the functionalizing step a) comprises treating the inert metal surface with a solution containing the surface agent at a concentration of about 1 μg/mL to about 50 μg/mL with a press load, preferably, the press load is a ceramic, glass or polymer load, which is applied across the whole substrate to result a stress from about 3-30 Pa. For example, the substrate can have a dimension of 100 mm in diameter, preferably 12.4 mm×25 mm.

Embodiment 7. The method of any of Embodiments 1-5, wherein the functionalizing step a) comprises treating a first area of the inert metal surface with a solution containing the surface agent at a concentration of about 0.1 μg/mL to about 15 μg/mL, such as using a CFM.

Embodiment 8. The method of Embodiment 7, further comprising treating a second area of the inert metal surface with a surface-agent-independent capture molecule, wherein the second area is different from the first area, wherein the surface-agent-independent capture molecule is capable of directly or indirectly binding to the inert metal surface without binding to the surface agent.

Embodiment 9. The method of any of Embodiments 1-8, wherein the blocking step b) comprises treating the surface-agent-functionalized surface with a first blocking solution comprising a modified polyethylene glycol (PEG), wherein the modified PEG is modified with an amine, carboxyl, or thiol at one end.

Embodiment 10. The method of Embodiment 9, wherein the modified PEG is a thiolated PEG, wherein the PEG is modified with a thiol at one end.

Embodiment 11. The method of Embodiment 10, wherein the thiolated PEG is capped with an alkoxy having 1-20 carbon atoms such as a methoxy at the other end.

Embodiment 12. The method of any of Embodiments 9-11, wherein the modified PEG has a number or weight average molecular weight, preferably, a number average molecular weight, of about 1000-5000 g/mol, about 1500-3000 g/mol, about 1750-2500 g/mol, preferably about 2000 g/mol.

Embodiment 13. The method of any of Embodiments 9-12, wherein the first blocking solution comprises the modified PEG at a concentration about 0.1-10 mM.

Embodiment 14. The method of any of Embodiments 9-13, wherein the blocking step b) further comprises treating the surface-agent-functionalized surface with a second blocking solution comprising a serum protein, wherein the second blocking solution is different from the first blocking solution, and the treatment with the second blocking solution occurs after the treatment with the first blocking solution.

Embodiment 15. The method of Embodiment 14, wherein the serum protein is albumin and/or fibrinogen, preferably, albumin.

Embodiment 16. The method of Embodiment 14, wherein the serum protein is albumin, such as bovine serum albumin.

Embodiment 17. The method of any of Embodiments 14-16, wherein the second blocking solution comprises the serum protein at a concentration of about 0.1% to about 5% (weight to volume, or “w/v”), such as about 1% (w/v).

Embodiment 18. The method of any of Embodiments 9-17, wherein the blocking step b) further comprises treating the surface-agent-functionalized surface with a third blocking solution comprising an antibody, preferably, the antibody is of the IgG isotype, wherein the third blocking solution is different from the first or second blocking solution, and the treatment with the third blocking solution occurs between the treatment with the first and second blocking solutions.

Embodiment 19. The method of Embodiment 18, wherein the antibody is a human antibody, a mouse antibody, and/or a rabbit antibody, for example, the third blocking solution comprises a mixture of human IgG and rabbit IgG.

Embodiment 20. The method of Embodiment 18, wherein the third blocking solution comprises a human IgG antibody at a concentration of about 10-300 μg/mL and a rabbit IgG antibody at a concentration of about 10-300 μg/mL, preferably, the molar ratio of the human IgG to the rabbit IgG ranges from about 0.1:10 to about 10:0.1, or any range or ration therein between, such as about 0.5:10, about 1:10, about 3:10, about 5:10, about 8:10, about 1:1, about 10:0.5, about 10:1, about 10:3, about 10:5, and about 10:8.

Embodiment 21. The method of any of Embodiments 9-20, wherein the blocking step b) further comprises treating the surface-agent-functionalized surface with a fourth blocking solution comprising a fragment crystallizable (Fc) region of an IgG antibody, such as a human, rabbit, or mouse IgG antibody, preferably, rabbit Fc region, for example, at a concentration of about 0.1 μg/mL to about 10 μg/mL.

Embodiment 22. The method of any of Embodiments 1-8, wherein the blocking step b) comprises treating the surface-agent-functionalized surface with a combined blocking solution comprising (i) a modified polyethylene glycol (PEG), wherein the modified PEG is modified with an amine, carboxyl, or thiol at one end; and (ii) a serum protein.

Embodiment 23. The method of Embodiment 22, wherein the modified PEG is a thiolated PEG, wherein the PEG is modified with a thiol at one end.

Embodiment 24. The method of Embodiment 23, wherein the thiolated PEG is capped with an alkoxy group having 1-20 carbon atoms, preferably 1 carbon atom (i.e., methoxy), at the other end.

Embodiment 25. The method of any of Embodiments 22-24, wherein the modified PEG has a number or weight average molecular weight, preferably, a number average molecular weight, of about 1000-5000 g/mol.

Embodiment 26. The method of any of Embodiments 22-25, wherein the combined blocking solution comprises the modified PEG at a concentration about 0.1-10 mM.

Embodiment 27. The method of any of Embodiments 22-26, wherein the serum protein is albumin and/or fibrinogen, preferably, albumin.

Embodiment 28. The method of any of Embodiments 22-26, wherein the serum protein is albumin, such as bovine serum albumin.

Embodiment 29. The method of any of Embodiments 22-28, wherein the combined blocking solution comprises the serum protein at a concentration of about 0.1% to about 5% (w/v), such as about 1% (w/v).

Embodiment 30. The method of any of Embodiments 22-29, wherein the combined blocking solution further comprises an antibody, preferably, the antibody is of the IgG isotype.

Embodiment 31. The method of Embodiment 30, wherein the antibody is a human antibody, a mouse antibody, and/or a rabbit antibody, for example, the combined blocking solution comprises a mixture of human IgG and rabbit IgG.

Embodiment 32. The method of Embodiment 30 or 31, wherein the combined blocking solution comprises a human IgG antibody at a concentration of about 10-300 μg/mL and a rabbit IgG antibody at a concentration of about 10-300 μg/mL, preferably, the molar ratio of the human IgG to the rabbit IgG ranges from about 0.1:10 to about 10:0.1.

Embodiment 33. The method of any of Embodiments 22-32, wherein the combined blocking solution further comprises a fragment crystallizable (Fc) region of an IgG antibody, such as a human, rabbit, or mouse IgG antibody, preferably, rabbit Fc region, for example, at a concentration of about 0.1 μg/mL to about 10 μg/mL.

Embodiment 34. The method of any of Embodiments 1-33, wherein the blocking step b) further comprising treating the surface-agent-functionalized surface with one or more ingredients selected from a buffer (e.g., phosphate buffered saline), a silane (e.g., decafluoro-1,1,2,2,-tetrahydrooctyl trichlorosilane (FOTS)), a surfactant (e.g., egg phosphatidylcholine, palmitoyl-oleoylphosphatidylcholine (POPC), Triton X. Tween 20, etc.), and a thiol (e.g., mercaptopropanol (MPO)).

Embodiment 35. The method of Embodiment 34, wherein the blocking step b) further comprising treating the surface-agent-functionalized surface with the buffer, preferably phosphate-buffered saline (PBS), sodium chloride-sodium phosphate-EDTA, or HEPES (4-(2-hydroxyethyl)-1-piperazineethanesulfonic acid), preferably, the buffer has a pH of about 6-8.

Embodiment 36. The method of Embodiment 34 or 35, wherein the blocking step b) further comprising treating the surface-agent-functionalized surface with the surfactant, such as Tween 20.

Embodiment 37. The method of any of Embodiments 1-36, further comprising treating the surface-agent-functionalized surface with a salt (e.g., sodium chloride) and/or a chelating agent (e.g., ethylenediaminetetraacetic acid (EDTA)).

Embodiment 38. The method of any of Embodiments 1-37, further comprising immobilizing a surface-agent-dependent capture molecule on the surface-agent-functionalized surface prior to the blocking step b), wherein the immobilizing comprises specifically binding the surface-agent-dependent capture molecule to the surface agent directly or indirectly.

Embodiment 39. The method of any of Embodiments 1-38, wherein the substrate is a glass, metal, ceramic, or polymer substrate, preferably a glass substrate.

Embodiment 40. The method of any of Embodiments 1-38, wherein the substrate is a glass substrate suitable for use in a surface plasmon resonance imaging analysis.

Embodiment 41. The substrate having the inert metal surface prepared by the method of any of Embodiments 1-40.

Embodiment 42. A combined blocking solution comprising: (a) a thiolated PEG, wherein the PEG is modified with a thiol at one end; (b) a serum protein; and optionally (c) an antibody, such as a human antibody, a mouse antibody, and/or a rabbit antibody.

Embodiment 43. The combined blocking solution of Embodiment 42, wherein the thiolated PEG is capped with an alkoxy having 1-20 carbon atoms such as a methoxy at the other end.

Embodiment 44. The combined blocking solution of Embodiment 42 or 43, wherein the thiolated PEG has a number or weight average molecular weight, preferably, a number average molecular weight, of about 1000-5000 g/mol.

Embodiment 45. The combined blocking solution of any of Embodiments 42-44, wherein the thiolated PEG is at a concentration about 0.1-10 mM.

Embodiment 46. The combined blocking solution of any of Embodiments 42-45, wherein the serum protein is albumin, such as bovine serum albumin.

Embodiment 47. The combined blocking solution of any of Embodiments 42-46, wherein the serum protein is bovine serum albumin, and the combined blocking solution comprises the bovine serum albumin at a concentration of about 0.1% to about 5% (w/v), such as about 1% (w/v).

Embodiment 48. The combined blocking solution of any of Embodiments 42-47, wherein the combined blocking solution comprises the antibody, such as a human antibody, a mouse antibody, and/or a rabbit antibody, for example, the combined blocking solution comprises a mixture of human IgG and rabbit IgG.

Embodiment 49. The combined blocking solution of Embodiment 48, wherein the combined blocking solution comprises a human IgG antibody at a concentration of about 10-300 μg/mL and a rabbit IgG antibody at a concentration of about 10-300 μg/mL, preferably, the molar ratio of human IgG to the rabbit IgG ranges from about 0.1:10 to about 10:0.1.

Embodiment 50. The combined blocking solution of any of Embodiments 42-49, further comprising a fragment crystallizable (Fc) region of an IgG antibody, such as a human, rabbit, or mouse IgG antibody, preferably, rabbit Fc region, for example, at a concentration of about 0.1 μg/mL to about 10 μg/mL.

Embodiment 51. The combined blocking solution of any of Embodiments 42-50, further comprising a buffer, such as phosphate-buffered saline (PBS), sodium chloride-sodium phosphate-EDTA, or HEPES (4-(2-hydroxyethyl)-1-piperazineethanesulfonic acid), at a pH of about 6-8.

Embodiment 52. A combination of blocking agents comprising (a) a first solution comprising a thiolated PEG, wherein the PEG is modified with a thiol at one end; (b) a second solution comprising a serum protein; and optionally (c) a third solution comprising an antibody, such as a human antibody, a mouse antibody, and/or a rabbit antibody, wherein the first, second, and third solution do not contain the same blocking agent(s).

Embodiment 53. The combination of Embodiment 52, wherein the thiolated PEG is capped with an alkoxy group having 1-20 carbon atoms, preferably, methoxy, at the other end.

Embodiment 54. The combination of Embodiment 52 or 53, wherein the thiolated PEG has a number or weight average molecular weight, preferably, a number average molecular weight, of about 1000-5000 g/mol.

Embodiment 55. The combination of any of Embodiments 52-54, wherein the first solution comprises the thiolated PEG at a concentration about 0.1-10 mM.

Embodiment 56. The combination of any of Embodiments 52-55, wherein the serum protein is albumin, such as bovine serum albumin.

Embodiment 57. The combination of any of Embodiments 52-55, wherein the serum protein is bovine serum albumin, and the second solution comprises the bovine serum albumin at a concentration of about 0.1% to about 5% (w/v), such as about 1% (w/v).

Embodiment 58. The combination of any of Embodiments 52-57, comprising the third solution, wherein the third solution comprises a human antibody, a mouse antibody, and/or a rabbit antibody, for example, the third solution comprises a mixture of human IgG and rabbit IgG.

Embodiment 59. The combination of Embodiment 58, wherein the third solution comprises a human IgG antibody at a concentration of about 10-300 μg/mL and a rabbit IgG antibody at a concentration of about 10-300 μg/mL, preferably, the molar ratio of human IgG to the rabbit IgG ranges from about 0.1:10 to about 10:0.1.

Embodiment 60. The combination of any of Embodiments 52-59 further comprising a fourth solution comprising a fragment crystallizable (Fc) region of an IgG antibody, such as a human, rabbit, or mouse IgG antibody, preferably, rabbit Fc region, for example, at a concentration of about 0.1 μg/mL to about 10 μg/mL.

Embodiment 61. The combination of any of Embodiments 52-60, wherein as applicable, the first, second, third, and fourth solution comprise a buffer, such as phosphate-buffered saline (PBS), sodium chloride-sodium phosphate-EDTA, or HEPES (4-(2-hydroxyethyl)-1-piperazineethanesulfonic acid), at a pH of about 6-8.

Embodiment 62. A substrate having an inert metal surface, wherein the inert metal surface is treated with any of the combined blocking solution of Embodiments 42-51 or any of the combination of any of Embodiments 52-61.

Embodiment 63. The substrate of Embodiment 62, wherein the inert metal surface is a gold surface, a silver surface, or a gold/silver alloy surface, preferably, a gold surface coated on the substrate.

Embodiment 64. A kit comprising (i) a substrate having an inert metal surface; and (ii) any of the combined blocking solution of Embodiments 42-51 or any of the combination of any of Embodiments 52-61.

Embodiment 65. The kit of Embodiment 64, wherein the inert metal surface is a gold, a silver surface, or a gold/silver alloy surface, preferably, a gold surface surface coated on the substrate.

Embodiment 66. The kit of Embodiment 64 or 65, further comprising a surface agent.

Embodiment 67. The kit of any of Embodiments 64-66, wherein the inert metal surface is functionalized with a surface agent.

Embodiment 68. The kit of Embodiment 66 or 67, further comprising a surface-agent-dependent capture molecule, wherein the surface agent is capable of binding to the inert metal surface and specifically binding to the surface-agent-dependent capture molecule, wherein the surface-agent-dependent capture molecule is capable of specifically binding to one or more analytes.

Embodiment 69. The kit of any of Embodiments 64-68, further comprising a surface-agent-independent capture molecule, wherein the surface-agent-independent capture molecule is capable of directly or indirectly binding to the inert surface without binding to a surface agent, and the surface-agent-independent capture molecule is capable of specifically binding to one or more analytes.

Embodiment 70. The kit of any of Embodiments 66-69, wherein the surface agent is a thiolated protein A, wherein the protein A is modified with a thiol.

Embodiment 71. The kit of any of Embodiments 64-70, comprising one or more ingredients selected from a buffer (e.g., phosphate buffered saline), a silane (e.g., decafluoro-1,1,2,2,-tetrahydrooctyl trichlorosilane (FOTS)), a surfactant (e.g., egg phosphatidylcholine, palmitoyl-oleoylphosphatidylcholine (POPC), Triton X. Tween 20, etc.), and a thiol (e.g., mercaptopropanol (MPO)).

Embodiment 72. The kit of any of Embodiments 64-71, comprising a buffer, preferably phosphate-buffered saline (PBS), sodium chloride-sodium phosphate-EDTA, or HEPES (4-(2-hydroxyethyl)-1-piperazineethanesulfonic acid), preferably, the buffer has a pH of about 6-8.

Embodiment 73. The kit of any of Embodiments 64-72, comprising a surfactant, such as Tween 20.

Embodiment 74. The kit of any of Embodiments 64-73, comprising a salt (e.g., sodium chloride) and/or a chelating agent (e.g., ethylenediaminetetraacetic acid (EDTA)).

Embodiment 75. The kit of any of Embodiments 64-74, further comprising one or more components selected from antigens, serum samples, inhibitors that stabilize a pathogen-related DNA or RNA target in the serum samples, detection molecules such as detection aptamers and detection antibodies, and nanoenhancers.

Embodiment 76. The kit of any of Embodiments 64-75, wherein the substrate is a glass substrate.

Embodiment 77. The kit of Embodiment 76, wherein the glass substrate is suitable for use in a surface plasmon resonance imaging analysis.

Embodiment 78. A substrate having an inert metal surface, wherein the inert metal surface comprises:
a) a surface-agent-dependent capture molecule, which is immobilized on the inert metal surface through specific binding to a surface agent bound to the inert metal surface; and
b) a plurality of blocking agents, which are bound to the inert metal surface directly or indirectly,
wherein the surface-agent-dependent capture molecule is capable of specifically binding to one or more analytes, wherein the plurality of blocking agents is capable of reducing (preferably substantially reducing) or preventing the inert metal surface from non-specific binding.

Embodiment 79. The substrate of Embodiment 78, wherein the surface agent is a protein, preferably, protein A, protein G, protein L, avidin, or streptavidin.

Embodiment 80. The substrate of Embodiment 79, wherein the surface agent is protein A modified with amine, carboxyl, hydroxyl, and/or thiol.

Embodiment 81. The substrate of Embodiment 79, wherein the surface agent is a thiolated protein A, wherein the protein A is modified with a thiol.

Embodiment 82. The substrate of any one of Embodiments 78-81, wherein the surface-agent-dependent capture molecule is a capture antibody.

Embodiment 83. The substrate of Embodiment 82, wherein the capture antibody is an IgG isotype antibody, and the surface agent is a thiolated protein A.

Embodiment 84. The substrate of any one of Embodiments 78-83, wherein the plurality of blocking agents comprise a modified polyethylene glycol (PEG), wherein the modified PEG is modified with an amine, carboxyl, or thiol at one end.

Embodiment 85. The substrate of Embodiment 84, wherein the modified PEG is a thiolated PEG, wherein the PEG is modified with a thiol at one end.

Embodiment 86. The substrate of Embodiment 85, wherein the thiolated PEG is capped with an alkoxy having 1-20 carbon atoms, preferably, methoxy, at the other end.

Embodiment 87. The substrate of any one of Embodiments 84-86, wherein the modified PEG has a number or weight average molecular weight, preferably, a number average molecular weight, of about 1000-5000 g/mol.

Embodiment 88. The substrate of any one of Embodiments 84-87, wherein the plurality of blocking agents further comprise a serum protein.

Embodiment 89. The substrate of Embodiment 88, wherein the serum protein is albumin and/or fibrinogen, preferably, albumin.

Embodiment 90. The substrate of Embodiment 89, wherein the serum protein is bovine serum albumin.

Embodiment 91. The substrate of any one of Embodiments 84-90, wherein the plurality of blocking agents further comprise an antibody, such as a human antibody, a mouse antibody, and/or a rabbit antibody, preferably, the antibody is of the IgG isotype.

Embodiment 92. The substrate of any one of Embodiments 84-90, wherein the plurality of blocking agents further comprise a mixture of human IgG and rabbit IgG.

Embodiment 93. The substrate of any one of Embodiments 84-92, wherein the plurality of blocking agents further comprise a fragment crystallizable (Fc) region of an IgG antibody, such as a human, rabbit, or mouse IgG antibody, preferably, rabbit Fc region.

Embodiment 94. The substrate of any one of Embodiments 84-93, wherein the plurality of blocking agents further comprise one or more ingredients selected from a buffer (e.g., phosphate buffered saline), a silane (e.g., decafluoro-1,1,2,2,-tetrahydrooctyl trichlorosilane (FOTS)), a surfactant (e.g., egg phosphatidylcholine, palmitoyl-oleoylphosphatidylcholine (POPC), Triton X, Tween 20, etc.), and a thiol (e.g., mercaptopropanol (MPO)).

Embodiment 95. The substrate of any one of Embodiments 78-94, wherein the surface agent is uniformly bound to the inert metal surface.

Embodiment 96. The substrate of any one of Embodiments 74-94, wherein the surface agent is bound to the inert metal surface at a predefined area.

Embodiment 97. The substrate of Embodiment 96, further comprising a surface-agent-independent capture molecule, directly or indirectly bound to the inert metal surface without binding to the surface agent, for example, the surface-agent-independent capture molecule is a capture aptamer.

Embodiment 98. The substrate of any one of Embodiments 74-97, wherein the inert metal surface is a gold surface, a silver surface, or a gold/silver alloy surface, preferably, a gold surface coated on the substrate.

Embodiment 99. The substrate of any one of Embodiments 74-98, which is a glass, metal, ceramic, or polymer substrate, preferably a glass substrate.

Embodiment 100. The substrate of any one of Embodiments 74-99, which is a glass substrate suitable for use in a surface plasmon resonance imaging analysis.

Embodiment 101. A method of analyzing a sample, comprising (a) providing the substrate of any one of Embodiments 41, 62, 63, and 78-100, wherein the substrate comprises at least one capture molecule on the inert metal surface that is capable of specifically binding to an analyte; (b) incubating the sample with the substrate under a condition suitable for the at least one capture molecule to specifically bind to the analyte; and (c) determining whether the sample specifically binds the substrate, thereby determining whether the analyte is present in the sample.

Embodiment 102. The method of Embodiment 101, wherein the determining step c) comprises comparing surface plasmon resonance reflectivity of the substrate incubated with the sample or a control.

It is to be understood that both the foregoing summary and the following detailed description are exemplary and explanatory only, and are not restrictive of the invention herein.

### BRIEF DESCRIPTION OF THE FIGURES

FIG. 1 shows a SPRi-based assay for detection of a biomarker IL4 in serum sample. The assay is run in the flow cell of SPRi instrument OpenPlex (inset: 48×49×30.4 cm, 29 lbs, 40 μL unpurified serum, <1 hr run time) from HORIBA Scientific. Each biomarker in serum is captured on the specific spot array and the concentration and the binding kinetics to its corresponding pair are determined from the reflectivity changes in the flow.

FIG. 2 shows Protein A treatments on the surface of a sensing chip. Left image shows a non-uniform protein A treatment without the coverslip while right image presents uniform treatment with the use of a coverslip since uniform force and resultant stress are exerted across the chip.

FIG. 3 shows percent reflectivity vs. time profiles (signal profiles) after treating three different sensing chips with blocking agents. The blocking agent signals monitor background, capture antibody and control antibody spots (n=5, each) on different sensing chips. The sensing chips are functionalized with Protein A with the use of a press load (resultant stress of 3 Pa). Points 1, 2, and 3 in each of A, B, and C are the maximum signals obtained after thiolated PEG, rabbit IgG/human IgG mix and BSA injections, respectively.

FIG. 4 shows signal profiles of sensing surface using chips: (A-B) functionalized with a press load and blocked, (C-D) functionalized without a press load and blocked. (E-F) functionalized with a heavy press load and blocked, (G) functionalized with a press load, (H-I) functionalized with a press load and partially blocked. Capture antibody=300 μg/mL; [PBS running buffer]=1×; [IL4]=10 ng/mL spiked in 10% human serum; [Biotinylated detection antibody]=10 nM; [streptavidin-coated QD]=10 nM.

FIG. 5 shows additional signal profiles of sensing surface with blocking agent signal monitoring of background, capture antibody and control antibody spots (n=5, each) on different sensing chips. The sensing chips are functionalized with Protein A without the use of a press load. Points 1, 2, and 3 in each of A and B are the maximum signals obtained after thiolated PEG, rabbit IgG/human IgG mix and BSA injections, respectively.

FIG. 6 shows a bar graph demonstrating reproducibility of the different conditions used to optimize the surface activation system herein on the SPRi chip surface. Delta value is a control-subtracted % reflectivity signal. S/B means signal to background ratio. For each condition, a cytokine IL4 (10 ng/mL) spiked in 10% human serum was used.

FIG. 7 shows a picture of different sample spotting techniques using a (A) manual Arrayer and a (B) continuous flow microspotter, CFM.

FIG. 8 shows a comparison of sensing array construction using (A) an Arrayer and (B) a CFM.

FIG. 9 presents a scheme of a SPRi-based assay for multiplex detection of ZIKV indicators RNA, IgM, and IgG. Surface functionalization and sensing array construction were done using Luna Labs' MultiSpot® MSprinting technology.

FIG. 10 shows a diagram of a SPRi-based assay for a sequential detection of ZIKV indicators IgM and IgG. These detections follow after ZIKV RNA identification.

FIG. 11 shows sensorgrams of ZIKV RNA, IgM, and IgG spiked in 10% human serum sample using sensing chips (A) without blocking. (B) with partial blocking. (C) with sequential blocking, and (D) with mixed blocking. Each line represents the average control-subtracted reflectivity (delta) value from 4 spot replicates. Letters indicate injection points of reagents (see Table 7) during multiplex detection of indicators.

FIG. 12 show spot images taken after QD addition for chips (A) without blocking and (B) with mixed blocking. Spots after each detection are indicated by arrows. ZCAp means ZIKV capture aptamer; C4 indicates control capture aptamer; 117 signifies ZIKV capture antibody; RIgG means control capture antibody (rabbit IgG).

FIG. 13 presents (A) K_{D} values of antibody/cytokine interactions using SAS in a SPRi platform. Binding affinity trend among cytokines analyzed: MIP4>IL13>IL18. FIG. 13 also presents (B) Multiplex detection of MIP4, IL13 and IL8 in buffer.

FIG. 14 shows difference images taken from the analyses of 50 ng/mL 23-nt long DNA (DmiR122) and 5 ng/mL protein (IL4) biomarkers in 10% serum using SAS on a single chip. *DmiR122 is a DNA counterpart of microRNA122, a biomarker for liver injury. LNA aptamers were used to capture and detect DmiR122

### DETAILED DESCRIPTION

The present disclosure generally relates to the preparation of functionalized surfaces, which can be useful for the analysis of biomarkers present in a wide variety of biofluids (e.g., serum, plasma, whole blood, urine, cerebrospinal fluid, etc.).

For all types of ligand binding assays for biomarker or indicator detection (i.e., biomarker assays), the accuracy and reliability of analysis depends largely on the signal-to-noise (S/N) ratio of the results. To achieve optimized detection signals, the surface functionalization, protein and DNA printing, and blocking can all be critical steps. If these are not implemented, the risk of compromising the reliability of assay results is high.

Functionalization is the foundation of the sensing assay construct. Without wishing to be bound by theories, the functionalization agent or surface agent (e.g., a thiolated protein A) effectively holds the capture antibody in proper orientation so it can readily and effectively bind to the antigen which in turn takes the indicator (e.g., target antibody) of interest for its subsequent detection.

Proper construction of sensing microarrays is of also high importance. The high-throughput capability of microarrays is very useful to examine a large repertoire of proteins and DNA of interest. Antibody and antigen arrays are used for disease biomarker identification and quantification, protein function determination, autoantibody detection, biomarker profiling, and characterization of protein-molecule interactions while DNA microarrays are used to simultaneously measure the expression levels of large numbers of genes associated to pathogens or diseases. However, achieving highly uniform spots from low concentrations of capture proteins and aptamer is difficult. Moreover, the introduction of multiple antigens in SPRi flow across the chip surface may cause cross-contamination of the immobilized antibodies (i.e., capture antibodies from different pathogens of interest) resulting in unreliable target indicator signals. To overcome this issue, spotting of antigen (antigen microarray) directly on top of the capture antibody instead of injecting antigens into the microfluidic chamber is favorable. With a MS (multiple spotting), the printing of high-quality spots from low-concentration solutions, and the prevention of cross-contamination among antigens during biomarker detection are feasible. MS technologies have the capability to construct combinatorial microarrays as they can be designed to concurrently print both nucleic acids (RNA/DNA) and proteins.

Surface blocking is equal in importance to sensing array construction. Blocking agents block all unoccupied sites on the surface to prevent the non-specific binding events especially when the biomarker/indicator analysis involves complex solutions or suspensions such as clinical serum and plasma samples. Human serum and plasma are heterogeneous samples that consist of abundant proteins, electrolytes, antibodies, antigens, hormones, and exogenous substances (e.g., drugs and microorganisms). Without blocking the assay surface, non-specific binding from these serum or plasma proteins can be too high and significantly hinder the detection of low abundant target biomarkers, making it difficult to assess the status of the diseases. Thus, by using the appropriate blocking agents, the S/N ratio can be improved while background signal can be significantly reduced, resulting in better detection sensitivity and precision.

In a variety of biological binding and detection applications, there is a need to modify sensing surfaces to ensure that only the desired biomacromolecules are bound and subsequently detected with high (S/N) ratio. The present disclosure describes a general surface activation, which includes for example, functionalization, printing or array construction, and blocking, which can be used for target biomacromolecule detection in biological samples such as human serum samples.

### Method of Preparing Surfaces

Some embodiments of the present disclosure are directed to methods of preparing surfaces. In some embodiments, a method of preparing a substrate having an inert metal surface is provided. In some embodiments, the method comprises (a) functionalizing the inert metal surface with a surface agent to produce a surface-agent-functionalized surface; and (b) blocking the inert metal surface to reduce or prevent non-specific binding. Typically, the blocking step (b) is subsequent to the functionalizing step (a). Typically, the inert metal surface is a gold surface coated on the substrate. In some embodiments, the inert metal surface can also be a silver surface or a gold/silver alloy surface. Suitable substrates are not particularly limited, which can be a glass, metal, ceramic, and/or polymer substrate, preferably a glass substrate. For example, in some preferred embodiments, the substrate can be a glass substrate having a gold surface, which is suitable for use in a SPR analysis. While many of the embodiments described herein are directed to preparing an inert metal surface, particularly gold surfaces, the present disclosure is not so limited. In particular, the present disclosure also provides methods for preparing other surfaces such as metals, glass, ceramics and polymers, wherein the methods can include the surface functionalization, array construction, and/or blocking described herein, except with the inert metal surface replaced with another surface. It should also be noted that the substrate having the inert metal surface prepared by any of the methods herein are also novel substrates of the present disclosure.

In some embodiments, the entire inert metal surface is treated with the surface agent, for example, in the presence of a press load or without a press load. As described herein, data shown herein suggests that surface functionalization using a press load can achieve a more uniform functionalization with the surface agent (e.g., thiolated protein A) across the surface (e.g., a gold surface) compared to those prepared without a press load. Useful press loads are not particularly limited, which for example can be a-ceramic, glass or polymer load, or a combination thereof. Typically, the press load is applied to the substrate having the inert metal surface to result a stress of about 3 Pa to about 30 Pa, such as about 3 Pa, about 5 Pa, about 15 Pa, about 20 Pa, about 30 Pa, or any ranges between the recited values. In some preferred embodiments, the press load can be a glass press load, which is applied to achieve a resultant stress from about 3 Pa to about 30 Pa, such as about 3 Pa, about 5 Pa, about 15 Pa, about 20 Pa, about 30 Pa, or any ranges between the recited value, more preferably about 3 Pa. The dimensions of the glass press load are also not limited. In some preferred embodiments, the press load is a glass cover slip having a dimension of 1″×1″ (25 mm×25 mm) and 0.5 mm in thickness.

In some embodiments, the inert metal surface is treated with the surface agent only at certain specified areas. For example, in some embodiments, the inert metal surface can be spotted with the surface agent. e.g., using an automated printer CFM to spot thiolated protein A on specific area of the inert metal surface (e.g., gold surface). Inert metal surfaces spotted with the surface agent may have different uses from those prepared by treating the entire surface with the surface agent. For example, in some embodiments, an inert metal surface spotted with the surface agent at different specific areas of the surface can be used for simultaneous antigen and DNA array printing, such as spot-on-spot printing, which can be used for microarray construction and analysis of multiple biomarkers on the same chip surface. To be clear, in such embodiments, the surface treatment is carried out without using a press load.

Suitable surface agents for functionalization are not particularly limited. The surface agent can be typically characterized as being capable of covalently binding to the inert metal surface, for example, the surface agent can contain a functional group such as an amine, carboxyl, hydroxyl, and/or thiol. In preferred embodiments, the surface agent can be a protein, preferably, protein A, protein G, protein L, avidin, or streptavidin or a fragment or functional variant thereof. For example, in some preferred embodiments, the surface agent can be protein A modified with amine, carboxyl, hydroxyl, and/or thiol. In any of the embodiments described herein, unless otherwise specified or contrary from context, the surface agent can be thiolated protein A, wherein the protein A is modified with a thiol.

In some embodiments, the surface agent specifically binds a protein containing an Fc region of an immunoglobulin. In some embodiments, the surface agent is a protein. In further embodiments, the surface agent is a protein selected from Protein A, Protein G, Protein L. Protein Z, Protein LG, Protein LA, Protein AG, or a fragment or functional variant thereof that is characterized by specifically binding to an Fc region of an immunoglobulin. In further embodiments, the surface agent is modified with amine, carboxyl, hydroxyl, and/or thiol.

In some embodiments, the surface agent is Protein A. In some embodiments, the surface agent comprises the sequence of SEQ ID NO: 1. In further embodiments, the surface agent is modified with amine, carboxyl, hydroxyl, and/or thiol.

In some embodiments, the surface agent is Protein A, or a fragment or functional variant thereof that is characterized by specifically binding to an Fc region of an immunoglobulin and comprising one or more Protein A homologous immunoglobulin binding domains selected from Protein A domain E, domain D, domain A, domain B, domain Z, and/or domain C. In some embodiments, the surface agent comprises 1, 2, 3, 4, 5, 6, 7, 8, 9. 10, 1-25, 1-15, or 2-10 Protein A homologous immunoglobulin binding domains. In further embodiments, the surface agent is modified with amine, carboxyl, hydroxyl, and/or thiol.

In some embodiments, the surface agent is Protein A, or a fragment or functional variant thereof that is characterized by specifically binding to an Fc region of an immunoglobulin and that comprises at least one amino acid sequence selected from: AQHDEAQQNAFYQVLNMPNLNADQRNGFIQSLKDDPSQSANVLGEAQKLNDSQAP (SEQ ID NO:2), ADAFNKDQQSAFYEILNMPNLNEAQRNGFIQSLKDDPSQSTNVLGE AKKLNESQAP (SEQ ID NO:3), ADNNFNKEQQNAFYEILNMPNLNEEQRNGFIQSLKD DPSQSANLLSEAKKLNESQAP (SEQ ID NO:4), ADNKFNKEQQNAFYEILHLPNLNE EQRNGFIQS LKDDPSQSANLLAEAKKLNDAQAP (SEQ ID NO:5), ADNKENKEQ QNAFYEILHLPNLTEEQRNGFIQSLKDDPSVSKEILAEAKKLNDAQAP (SEQ ID NO: 6), and VDNKFNKEQQNAFYEILHLPNLNEEQRNAFIQSLKDDPSQSANLLAE AKKLNDAQAP (SEQ ID NO: 7). In some embodiments, the surface agent comprises the amino acid sequence FNMQQQRRFYEALHDPNLNEEQRNAKIKSIRDD (SEQ ID NO: 8). In further embodiments, the surface agent is modified with amine, carboxyl, hydroxyl, and/or thiol.

In some embodiments, the surface agent is a Protein A fragment or functional variant that is characterized by specifically binding to an Fc region of an immunoglobulin and that contains an amino acid sequence having at least 80%, 85%, 90%, 95%, 97%, 98% or 99% sequence identity to the amino acid sequence of SEQ ID NO:1. In further embodiments, the surface agent is modified with amine, carboxyl, hydroxyl, and/or thiol.

In some embodiments, the surface agent is a Protein A fragment or functional variant that is characterized by specifically binding to an Fc region of an immunoglobulin and that has an amino acid sequence that contains 1, 2, 3, 4, 5, 6, 7, 8, 9, 10, 1-25, 1-15, or 2-10, additions, substitutions, and/or deletions compared to a reference amino acid sequence of SEQ ID NO: 1. In some embodiments, the Protein A fragment or functional variant has an amino acid sequence that contains 1, 2, 3, 4, 5, 6, 7, 8, 9, 10, 1-25, 1-15, or 2-10, additions compared to a reference amino acid sequence of SEQ ID NO: 1. In some embodiments, the Protein A fragment or functional variant has an amino acid sequence that contains 1, 2, 3, 4, 5, 6, 7, 8, 9, 10, 1-25. 1-15, or 2-10, substitutions compared to a reference amino acid sequence of SEQ ID NO: 1. In some embodiments, the Protein A fragment or functional variant has an amino acid sequence that contains 1, 2, 3, 4, 5, 6, 7, 8, 9, 10, 1-25, 1-15, or 2-10 deletions compared to a reference amino acid sequence of SEQ ID NO: 1. In further embodiments, the surface agent is modified with amine, carboxyl, hydroxyl, and/or thiol.

In some embodiments, the surface agent is Protein G. In some embodiments, the surface agent comprises the sequence of SEQ ID NO: 9. In further embodiments, the surface agent is modified with amine, carboxyl, hydroxyl, and/or thiol.

In some embodiments, the surface agent is a Protein G, or a fragment or functional variant that is characterized by specifically binding to an Fc region of an immunoglobulin and comprising one or more Protein G homologous immunoglobulin binding domains selected from protein G domain 1, domain 2, and/or domain 3. In some embodiments, the surface agent comprises 1, 2, 3, 4, 5, 6, 7, 8, 9, 10, 1-25, 1-15, or 2-10 Protein G homologous immunoglobulin binding domains. In further embodiments, the surface agent is modified with amine, carboxyl, hydroxyl, and/or thiol.

In some embodiments, the surface agent is Protein G, or a fragment or functional variant thereof that is characterized by specifically binding to an Fc region of an immunoglobulin and that comprises at least one amino acid sequence selected from: YKLILNGKTLKGETTTEAVDAATAEKVFKQYANDNGVDGEWTYDDATKTFTVTE (SEQ ID NO:10), TYKLVINGKTLKGETTTEAVDAATAEKVFKQYANDNGVDGEWT YDDATKTFTVTE (SEQ ID NO:11), and TYKLVINGKTLKGETTTKAVDAETAEKAF KQYANDNGVDGVWTYDDATKTFTVTE (SEQ ID NO:12). In further embodiments, the surface agent is modified with amine, carboxyl, hydroxyl, and/or thiol.

In some embodiments, the surface agent is a Protein G fragment or functional variant that is characterized by specifically binding to an Fc region of an immunoglobulin and that contains an amino acid sequence that has at least 80%, 85%, 90%, 95%, 97%, 98% or 99% sequence identity to the amino acid sequence of SEQ ID NO: 9. In further embodiments, the surface agent is modified with amine, carboxyl, hydroxyl, and/or thiol.

In some embodiments, the surface agent is a Protein G fragment or functional variant that is characterized by specifically binding to an Fc region of an immunoglobulin and that has an amino acid sequence that contains 1, 2, 3, 4, 5, 6, 7, 8, 9, 10, 1-25, 1-15, 2-10, additions, substitutions, and/or deletions compared to a reference amino acid sequence of SEQ ID NO:9. In some embodiments, the Protein G fragment or functional variant has an amino acid sequence that contains 1, 2, 3, 4, 5, 6, 7, 8, 9, 10, 1-25, 1-15, or 2-10, additions compared to a reference amino acid sequence of SEQ ID NO:9. In some embodiments, the Protein G fragment or functional variant has an amino acid sequence that contains 1, 2, 3, 4, 5, 6. 7. 8, 9, 10, 1-25. 1-15, or 2-10, substitutions compared to a reference amino acid sequence of SEQ ID NO: 9. In some embodiments, the Protein G fragment or functional variant has an amino acid sequence that contains 1, 2, 3, 4, 5, 6, 7, 8, 9, 10, 1-25, 1-15, or 2-10 deletions compared to a reference amino acid sequence of SEQ ID NO:9. In further embodiments, the surface agent is modified with amine, carboxyl, hydroxyl, and/or thiol.

In some embodiments, the surface agent is Protein L. In some embodiments, the surface agent comprises the sequence of SEQ ID NO: 13. In some embodiments, the surface agent is Protein L and comprises the sequence of SEQ ID NO: 13. In further embodiments, the surface agent is modified with amine, carboxyl, hydroxyl, and/or thiol.

In some embodiments, the surface agent is Protein L, or a fragment or functional variant thereof that is characterized by specifically binding to an Fc region of an immunoglobulin and comprising one or more Protein L homologous immunoglobulin binding domains selected from Protein L immunoglobulin binding domain B1, B2, B3, B4, and/or B5. In some embodiments, the surface agent comprises 1, 2, 3, 4, 5, 6. 7, 8, 9, 10, 1-25, 1-15, or 2-10 Protein L homologous immunoglobulin binding domains. In further embodiments, the surface agent is modified with amine, carboxyl, hydroxyl, and/or thiol.

In some embodiments, the surface agent is Protein L or a fragment or functional variant thereof that is characterized by specifically binding to an Fc region of an immunoglobulin and that comprises at least one Protein L amino acid sequence selected from: KEETPETPETDSEEEVTIKANLIFANGSTQTAEFKGTFEKATSEAYAYADTLKK DNGEYTVDVADKGYTLNIKFAG (SEQ ID NO:14); KEKTPEEPKEEVTIKANLIYADG KTQTAEFKGTFEEATAEAYRYADALKKDNGEYTVDVADKGYTLNIKFAG (SEQ ID NO: 15); KEKTPEEPKEEVTIKANLIYADGKTQTAEFKGTFEEATAEAYRYADLLA KENGKYTVDVADKGYTLNIKFAG (SEQ ID NO:16); and KEKTPEEPKEEVTIKA NLIYADGKTQTAEFKGTFAEATAEAYRYADLLAKENGKYTADLEDGGYTINIRFAG (SEQ ID NO:17). In further embodiments, the surface agent is modified with amine, carboxyl, hydroxyl, and/or thiol.

In some embodiments, the surface agent is a Protein L fragment or functional variant that is characterized by specifically binding to an Fc region of an immunoglobulin and that contains an amino acid sequence that has at least 80%, 85%, 90%, 95%, 97%, 98% or 99% sequence identity to the amino acid sequence of SEQ ID NO: 13. In further embodiments, the surface agent is modified with amine, carboxyl, hydroxyl, and/or thiol.

In some embodiments, the surface agent is a Protein L fragment or functional variant that is characterized by specifically binding to an Fc region of an immunoglobulin and that has an amino acid sequence that contains 1, 2, 3, 4, 5, 6, 7, 8, 9, 10, 1-25, 1-15, or 2-10, additions, substitutions, and/or deletions compared to a reference amino acid sequence of SEQ ID NO: 13. In some embodiments, the Protein L fragment or functional variant has an amino acid sequence that contains 1, 2, 3, 4, 5, 6, 7, 8, 9, 10, 1-25, 1-15, or 2-10, additions compared to a reference amino acid sequence of SEQ ID NO: 13. In some embodiments, the Protein L fragment or functional variant has an amino acid sequence that contains 1, 2, 3, 4, 5, 6, 7, 8, 9, 10, 1-25, 1-15, or 2-10, substitutions compared to a reference amino acid sequence of SEQ ID NO: 13. In some embodiments, the Protein L fragment or functional variant has an amino acid sequence that contains 1, 2, 3, 4, 5, 6, 7, 8, 9, 10, 1-25, 1-15, or 2-10 deletions compared to a reference amino acid sequence of SEQ ID NO: 13. In further embodiments, the surface agent is modified with amine, carboxyl, hydroxyl, and/or thiol.

In some embodiments, the surface agent specifically binds a protein containing an Fc region of an immunoglobulin and comprises at least one amino acid sequence selected from: WQRHGI (SEQ ID NO:18), MWRGWQ (SEQ ID NO:19), RHLGWF (SEQ ID NO:20), GWLHQR (SEQ ID NO:21), EPIHRSTLTALL (SEQ ID NO:22), HWRGWV (SEQ ID NO: 23), HYFKFD (SEQ ID NO:24), HFRRHL (SEQ ID NO:25), HWCGWV (SEQ ID NO: 26), RWHYFK (SEQ ID NO:27), WFRHYK (SEQ ID NO:28), NKFRGKYK, (SEQ ID NO: 29), NARKFYKG, (SEQ ID NO:30), FYWHCLDE (SEQ ID NO:31), FYCHWALE, (SEQ ID NO:32), FYCHTIDE (SEQ ID NO:33), RRGW (SEQ ID NO:34). KHRFNKD, (SEQ ID NO: 35), RWHYFK (SEQ ID NO:36), DCAWHLGELVWCT (SEQ ID NO:37) and TWKTSRISIF (SEQ ID NO:38). In further embodiments, the surface agent is modified with amine, carboxyl, hydroxyl, and/or thiol.

In some embodiments, the surface agent specifically binds a protein containing an Fc region of an immunoglobulin and comprises at least one of: D2AAG; DAAG; PAM; d-PAM; d-PAM-Φ; TWKTSRISIF 3 or 9; Fc-III; Fc-III, 3.5; Fc-III-4C, 3.5; FcRM, FGRLVSSIRY, Fc-III, FcBP-2, and Fc-III-4C. In further embodiments, the surface agent is modified with amine, carboxyl, hydroxyl, and/or thiol.

In some embodiments, the surface agent specifically binds a protein containing an Fc region of an immunoglobulin and comprises at least one of: D2AAG; DAAG; PAM; d-PAM; d-PAM-¢; TWKTSRISIF 3 or 9; Fc-III, 3.5; Fc-III-4C, 3.5: FcRM, FGRLVSSIRY, and Fc-III. In further embodiments, the surface agent is modified with amine, carboxyl, hydroxyl, and/or thiol.

In some embodiments, the surface agent specifically binds to biotin or a biotin compound. In some embodiments, the surface agent is a protein. In further embodiments, the surface agent is a protein selected from avidin, streptavidin, traptavidin, tamavidin, extravidin, and neutravidin, or a fragment or functional variant thereof that is characterized by specifically binding to biotin or a biotin compound. In further embodiments, the surface agent is modified with amine, carboxyl, hydroxyl, and/or thiol.

In some embodiments, the surface agent is streptavidin. In further embodiments, the surface agent comprises the sequence of SEQ ID NO: 39. In further embodiments, the surface agent is modified with amine, carboxyl, hydroxyl, and/or thiol.

In some embodiments, the surface agent is streptavidin, or a fragment or functional variant thereof that is characterized by specifically binding to biotin or a biotin compound and comprises the amino acid sequence of SEQ ID NO:40. In further embodiments, the surface agent is modified with amine, carboxyl, hydroxyl, and/or thiol.

In some embodiments, the surface agent is streptavidin, or a fragment or functional variant thereof that is characterized by specifically binding to biotin or a biotin compound and that contains an amino acid sequence having at least 80%, 85%, 90%, 95%, 97%, 98% or 99% sequence identity to the amino acid sequence of SEQ ID NO: 40. In further embodiments, the surface agent is modified with amine, carboxyl, hydroxyl, and/or thiol.

In some embodiments, the surface agent is streptavidin, or a fragment or functional variant thereof that is characterized by specifically binding to biotin or a biotin compound and that has an amino acid sequence that contains 1, 2, 3, 4, 5, 6, 7, 8, 9, 10, 1-25, 1-15, or 2-10, additions, substitutions, and/or deletions compared to a reference amino acid sequence of SEQ ID NO: 40. In some embodiments, the streptavidin, or fragment or functional variant thereof has an amino acid sequence that contains 1, 2, 3, 4, 5, 6, 7, 8. 9, 10, 1-25, 1-15, or 2-10, additions compared to a reference amino acid sequence of SEQ ID NO: 40. In some embodiments, the streptavidin, or fragment or functional variant thereof has an amino acid sequence that contains 1, 2, 3, 4, 5, 6, 7, 8, 9, 10. 1-25, 1-15, or 2-10, substitutions compared to a reference amino acid sequence of SEQ ID NO:40. In some embodiments, the streptavidin, or fragment or functional variant thereof has an amino acid sequence that contains 1, 2, 3, 4, 5, 6, 7, 8, 9, 10, 1-25, 1-15, or 2-10 deletions compared to a reference amino acid sequence of SEQ ID NO:40. In further embodiments, the surface agent is modified with amine, carboxyl, hydroxyl, and/or thiol.

In some embodiments, the surface agent is avidin. In further embodiments, the surface agent comprises the sequence of SEQ ID NO:41. In some embodiments, the surface agent is nonglycosylated and/or contains an N-Acyl moiety (e.g., an N-acetyl, N-phthalyl and N-succinyl moiety). In further embodiments, the surface agent is modified with amine, carboxyl, hydroxyl, and/or thiol.

In some embodiments, the surface agent is avidin, or a fragment or functional variant thereof that is characterized by specifically binding to biotin or a biotin compound and comprises the amino acid sequence of SEQ ID NO:41. In some embodiments, the surface agent is nonglycosylated and/or contains an N-Acyl moiety (e.g., an N-acetyl. N-phthalyl and N-succinyl moiety). In further embodiments, the surface agent is modified with amine, carboxyl, hydroxyl, and/or thiol.

In some embodiments, the surface agent is avidin, or a fragment or functional variant thereof that is characterized by specifically binding to biotin or a biotin compound and that contains an amino acid sequence having at least 80%, 85%, 90%, 95%, 97%, 98% or 99% sequence identity to the amino acid sequence of SEQ ID NO:41. In some embodiments, the surface agent is nonglycosylated and/or contains an N-Acyl moiety (e.g., an N-acetyl, N-phthalyl and N-succinyl moiety). In further embodiments, the surface agent is modified with amine, carboxyl, hydroxyl, and/or thiol.

In some embodiments, the surface agent is avidin, or a fragment or functional variant thereof that is characterized by specifically binding to biotin or a biotin compound and that has an amino acid sequence that contains 1, 2, 3, 4, 5, 6, 7, 8, 9, 10, 1-25, 1-15, or 2-10, additions, substitutions, and/or deletions compared to a reference amino acid sequence of SEQ ID NO:41. In some embodiments, the avidin, or fragment or functional variant thereof has an amino acid sequence that contains 1, 2, 3, 4, 5, 6, 7, 8, 9, 10, 1-25, 1-15, or 2-10, additions compared to a reference amino acid sequence of SEQ ID NO:41. In some embodiments, the avidin, or fragment or functional variant thereof has an amino acid sequence that contains 1, 2, 3, 4, 5, 6, 7, 8, 9, 10. 1-25, 1-15, or 2-10, substitutions compared to a reference amino acid sequence of SEQ ID NO:41. In some embodiments, the avidin, or fragment or functional variant thereof has an amino acid sequence that contains 1, 2, 3, 4, 5, 6, 7, 8. 9, 10, 1-25, 1-15, or 2-10 deletions compared to a reference amino acid sequence of SEQ ID NO:41. In some embodiments, the surface agent is nonglycosylated and/or contains an N-Acyl moiety (e.g., an N-acetyl, N-phthalyl and N-succinyl moiety). In further embodiments, the surface agent is modified with amine, carboxyl, hydroxyl, and/or thiol

In some embodiments, the surface agent comprises the amino acid sequence of SEQ ID NO: 42. In some embodiments, the surface agent is nonglycosylated and/or contains an N-Acyl moiety (e.g., an N-acetyl, N-phthalyl and N-succinyl moiety). In further embodiments, the surface agent is modified with amine, carboxyl, hydroxyl, and/or thiol.

In some embodiments, the surface agent comprises the amino acid sequence of SEQ ID NO: 43. In some embodiments, the surface agent is nonglycosylated and/or contains an N-Acyl moiety (e.g., an N-acetyl, N-phthalyl and N-succinyl moiety). In further embodiments, the surface agent is modified with amine, carboxyl, hydroxyl, and/or thiol.

Typically, for the methods herein, the inert metal surface is treated with a solution containing the surface agent. The concentration of the surface agent in the solution can vary. For example, in some embodiments, the entire inert metal surface is treated with the solution comprising the surface agent at a concentration ranging from about 5 μg/mL to about 50 μg/mL (e.g., about 5 μg/mL, about 15 μg/mL, about 25 μg/mL, about 35 μg/mL, about 50 μg/mL, or any range between the recited values) with a press load (e.g., described herein).

In some embodiments, a first area of the inert metal surface is treated with the solution comprising the surface agent at a concentration ranging from about 0.1 μg/mL to about 15 μg/mL (e.g., about 0.1 μg/mL, about 0.5 μg/mL, about 1 μg/mL, about 2 μg/mL, about 5 μg/mL, about 10 μg/mL, about 15 μg/mL, or any range between the recited values). In some embodiments, the inert metal surface is spotted with the solution comprising the surface agent at a concentration ranging from about 0.1 μg/mL to about 15 μg/mL (e.g., about 0.1 μg/mL, about 0.5 μg/mL, about 1 μg/mL, about 2 μg/mL, about 5 μg/mL, about 10 μg/mL, about 15 μg/mL, or any range between the recited values). The size of the first area is not particularly limited, which can be a typical spot size or a larger area. Typically, when the treatment of the surface is spotting the surface agent on particular positions, such as spotting protein A on a gold sensing chip surface, the spot size can range from smaller spots such as those having a dimension of 100 μm by 100 μm to larger spots with a dimension of 500 μm to 800 μm. In some embodiments, the spot size can have a dimension around 350 μm by 500 μm.

In some embodiments, the inert metal surface can be treated with a capture molecule prior to the treatment with the surface agent. For example, in some embodiments, the inert metal surface can be treated with a surface-agent-independent capture molecule, which is then followed by treating the entire inert metal surface with the surface agent as described herein, e.g., with a press load herein.

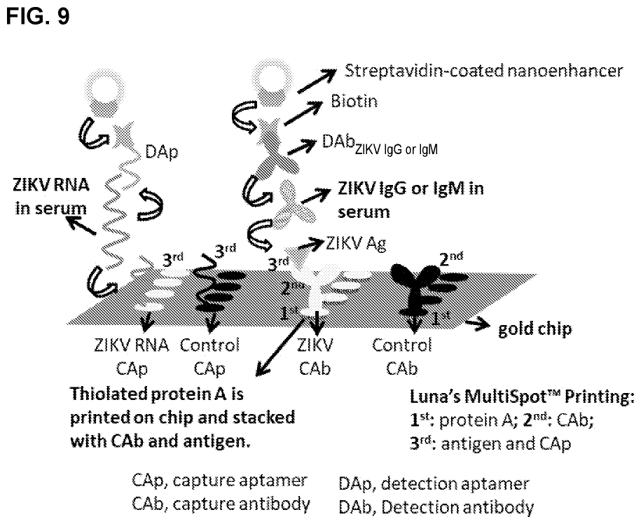

However, in preferred embodiments where a surface-agent-independent capture molecule is desired, the inert metal surface can be prepared by treating a first area of the inert metal surface with the surface agent as described herein, and treating a second area of the inert metal surface with a surface-agent-independent capture molecule, wherein the second area is different from the first area, wherein the surface-agent-independent capture molecule is capable of directly or indirectly binding to the inert metal surface without binding to the surface agent. As shown in FIG. 9, such methods can be advantageous at least in that they provide faster sample spotting, cleaner surface (no surface agents, e.g., protein A, over the surface-agent-independent capture molecule, e.g., capture aptamer), which can be fully automated and can have high quality of spot immobilization.

Suitable surface-agent-independent capture molecules for the methods herein are not particularly limited. For example, in some embodiments, the surface-agent-independent capture molecule can be a capture aptamer, preferably nucleic acids specific to biomarker targets associated with pathogens, organ injuries, skin diseases, and/or psychiatric disorder, preferably capture aptamers specific to DNA/RNA targets associated with Zika, Dengue, and/or Chikungunya viruses, or liver, kidney, brain, lung, or post-traumatic stress disorder (PTSD) biomarkers, more preferably DNA aptamers. The capture aptamers are preferably modified capture aptamers, such as with amine, carboxyl, hydroxyl, and/or thiol modifications, more preferably of thiol modification, such that they can be bound to the surface herein. When modified, the capture aptamers can have modifications at 5′ and/or 3′ ends, more preferably at 5′ end. The capture aptamers are preferably of 15-60 nucleotides long, more preferably 30 nucleotides. When used for treating the surface herein, the capture aptamer is preferably in a solution at a concentration ranging from about 50 μg/mL to about 1000 μg/mL, such as about 50 μg/mL, about 100 μg/mL, about 200 μg/mL, about 500 μg/mL, about 1000 μg/mL, or any range between the recited values, such as about 100 μg/mL. In some embodiments, the capture aptamer can be spotted onto an inert metal surface of the substrate, such as a gold film of a sensing chip, using a manual arrayer. In some embodiments, the capture aptamer can be spotted onto an inert metal surface of the substrate, such as a gold film of a sensing chip, using a microspotter. The concentrations of capture aptamer for use with a microspotter can be typically lower, such as in the range of about 5 μg/mL to about 150 μg/mL, such as about 5 μg/mL, about 10 μg/mL, about 15 μg/mL, about 20 μg/mL, about 50 μg/mL, about 75 μg/mL, about 100 μg/mL, about 125 μg/mL, about 150 μg/mL or any range or value between the recited values, such as about 10 μg/mL to about 20 μg/mL and about 12.5 μg/mL.

In some embodiments, the surface-agent-independent capture molecule can be a capture aptamer selected from nucleic acids with or without structure modifications. For example, in some preferred embodiments, the capture aptmer can be selected from those nucleic acids with structural modifications, such as locked nucleic acids and peptide nucleic acids, more preferably locked nucleic acids. When the structures of the nucleic acids are modified, the modification is typically for increasing melting temperature and/or strengthening the nucleic acids' binding with targets such as mRNA, DNA, RNA of shorter sequences, preferably mRNA of 18nt-60 nt long. In some preferred embodiments, the capture aptamer can be selected from single-stranded and double-stranded aptamers, more preferably single-stranded aptamers, preferably 5 nt-60 nt long, more preferably 12 nt long. The concentrations of the capture aptamer for use herein can preferably be in the range of 1 μg/mL-300 μg/mL, 2 μg/mL-200 μg/mL. 3 μg/mL-100 μg/mL, 4 μg/mL-75 μg/mL, 5 μg/mL-50 μg/mL. 5 μg/mL-25 μg/mL, such as about 5 μg/mL, about 10 μg/mL, about 15 μg/mL, or any range between the recited values, such as about 15 μg/mL. In some embodiments, the capture aptamer can be spotted onto an inert metal surface of the substrate, such as a gold film of a sensing chip, using a manual arrayer.

Typically, after being treated with the surface agent, regardless whether the entire inert metal surface or only specified areas of the inert metal surface is treated, the inert metal surface becomes a surface-agent-functionalized surface, which can be used to immobilize a surface-agent-dependent capture molecule on the surface-agent-functionalized surface. For example, in preferred embodiments, the method herein comprises immobilizing a surface-agent-dependent capture molecule on the surface-agent-functionalized surface prior to the blocking step b), wherein the immobilizing comprises specifically binding the surface-agent-dependent capture molecule to the surface agent directly or indirectly. In some embodiments, the immobilizing step can include spot-on-spot printing. For example, in some embodiments, the methods herein can comprise printing a thiolated protein A onto a gold film surface, which is followed by adding a capture antibody (CAb) via a spot-on-spot print, and adding antigen on top of CAb, optionally with simultaneous spotting with thiolated capture aptamer on the metal chip surface. Using this method, capture probes in both nucleic acid (RNA/DNA) and protein (antibodies, antigens) forms can be printed simultaneously on a chip.

Suitable surface-agent-dependent capture molecules for the methods herein are also not particularly limited. The surface-agent-dependent capture molecules can specifically bind to the surface agent either directly or indirectly. In some embodiments, the surface-agent-dependent capture molecules can be a capture antibody, such as antibodies against diseases such as infectious diseases, organ injuries, and/or skin diseases, preferably antibodies against Zika, Dengue, and/or Chikungunya viruses, liver, kidney, brain, or lung biomarkers, cytokines related to atopic dermatitis and/or exosomes associated with pancreatic cancer, etc. In some embodiments, the capture antibody can be antibodies of different isotypes IgM, IgG, IgD, IgA, or IgE, preferably of IgG isotype, preferably IgG with Fc region that can bind with protein A to afford an upright antibody orientation. When used for printing on the surface herein, the capture antibody is preferably in a solution with a concentration ranging from about 50 μg/mL to about 1000 μg/mL, such as about 50 μg/mL, about 100 μg/mL, about 200 μg/mL, about 500 μg/mL, about 1000 μg/mL, or any range between the recited values, such as about 100 μg/mL. In some embodiments, the capture antibody can be spotted onto an inert metal surface of the substrate, such as a gold film of a sensing chip, using a manual arrayer. In some embodiments, the capture antibody can be spotted onto an inert metal surface of the substrate, such as a gold film of a sensing chip, using a microspotter. The concentrations of capture antibody for use with a microspotter can be typically lower, such as in the range of about 0.1 μg/mL to about 50 μg/mL, such as about 1 μg/mL, about 10 μg/mL, about 15 μg/mL, about 20 μg/mL, about 50 μg/mL, or any range between the recited values, such as about 12 μg/mL or about 25 μg/mL.

In some embodiments, the surface-agent-dependent capture molecules can specifically bind to the surface agent indirectly. For example, in some embodiments, the surface-agent-dependent capture molecule can specifically bind to a capture antibody described herein, which can specifically bind to the surface-agent, such as through an Fc region that binds with protein A to afford an upright antibody orientation. In some embodiments, the surface-agent-dependent capture molecule can be an antigen, such as those specifically bind with antibodies against infectious diseases, such as Zika, Dengue, and/or Chikungunya viruses, those that are related to liver, kidney, brain, and/or lung diseases, those that are cytokines related to atopic dermatitis, those that are exosomes associated with pancreatic cancer. Concentrations of such antigens are not particularly limited. For example, when used in conjunction with a microspotter, such as spotted onto a gold film of a sensing chip using a microspotter, the antigen can have a concentration preferably in the range of about 5 μg/mL to about 100 μg/mL, more preferably about 2.5 μg/mL. In some embodiments, the antigen can be added during an analysis of the biosamples or can be spotted on top of a capture antibody on the surface, which can have a concentration of about 50 μg/mL to about 500 μg/mL, more preferably about 350 μg/mL.

As discussed herein, the methods herein typically include blocking the surface-agent-functionalized surface to reduce or prevent the inert metal surface from non-specific binding, such as those non-specific binding from proteins in a biological sample. The blocking step of the present disclosure can include treating the surface-agent-functionalized surface, which optionally contains immobilized capture molecule(s) as described herein, with one or more blocking solutions. When more than one blocking solutions are used, the blocking solutions each typically contains a different blocking agent or a different mixture of blocking agents, which can be used to treat the surface concurrently or sequentially in any order. In some embodiments, a combined blocking solution can optionally contain a mixture of all intended blocking agents.

Without wishing to be bound by theories, the blocking system described herein typically can include a blocking agent, such as a thiolated polyethylene glycol (PEG), that can be tethered onto the remaining surface that is not covered by the surface agent, such as gold layer that is not covered by thiolated protein A; and a blocking agent, such as IgG, BSA and/or Fc fragment, which can block the exposed regions of (i) the surface agent such as protein A, and (ii) molecules that are immobilized through binding to the surface agent, such as capture antibody, antigen, and/or control antibody. Thus, after blocking, only capture probe spots specific for target biomarker or indicator are allowed to interact. Target indicators will then be detected and amplified.

In some embodiments, the blocking step b) comprises treating the surface-agent-functionalized surface with a first blocking solution comprising a modified polyethylene glycol (PEG), wherein the modified PEG is modified with an amine, carboxyl, or thiol at one end. Preferably, the modified PEG is a thiolated PEG, wherein the PEG is modified with a thiol at one end. In some embodiments, the thiolated PEG is capped with an alkoxy group (with 1-20 carbon atoms), preferably methoxy, at the other end. In some specific embodiments, the thiolated PEG can be methoxypolyethylene glycol thiol characterized as having a general formula of CH₃O(CH₂CH₂O)ₙCH₂CH₂SH. The molecular weight of the modified PEG is not particularly limited. However, in some preferred embodiments, the modified PEG (such as the thiolated PEG) has a number or weight average molecular weight, preferably, a number average molecular weight, of about 1000 g/mol to about 5000 g/mol, such as about 1000 g/mol, about 2000 g/mol, about 3000 g/mol, about 4000 g/mol, about 5000 g/mol, or any range between the recited values. In some embodiments, the modified PEG can have a number average molecular weight of about 2000 g/mol. The first blocking solution typically comprises the modified PEG at a concentration about 0.1-10 mM. In some specific embodiments, the modified PEG can have a concentration of about 2 mM. In some specific embodiments, the modified PEG can have a concentration of about 4 mg/ml.

In some embodiments, the blocking step b) comprises treating the surface-agent-functionalized surface with a second blocking solution comprising a serum protein, wherein the second blocking solution is different from the first blocking solution, and the treatment with the second blocking solution occurs after the treatment with the first blocking solution. In some embodiments, the serum protein is albumin and/or fibrinogen, preferably, albumin. In some embodiments, the serum protein is albumin, such as bovine serum albumin. The second blocking solution typically comprises the serum albumin at a concentration ranging from about 0.1% to about 5% (w/v), such as about 0.1%, about 1%, about 2%, about 5%, or any range between the recited values. In some preferred embodiments, the second blocking solution comprises the serum protein at a concentration of about 1% (w/v). For example, in some embodiments, the second blocking solution comprises bovine serum albumin at a concentration of about 1% (w/v).

In some embodiments, the blocking step b) comprises treating the surface-agent-functionalized surface with a third blocking solution comprising an antibody, preferably, the antibody is of the IgG isotype, wherein the third blocking solution is different from the first or second blocking solution, and the treatment with the third blocking solution occurs between the treatment with the first and second blocking solutions. In some embodiments, the antibody is a human antibody, a mouse antibody, and/or a rabbit antibody, for example, the third blocking solution comprises a mixture of human IgG and rabbit IgG. In some embodiments, the third blocking solution comprises a human IgG antibody at a concentration of about 10-300 μg/mL (e.g., about 10 μg/mL, about 100 μg/mL, about 300 μg/mL, or any range between the recited values) and a rabbit IgG antibody at a concentration of about 10-300 μg/mL (e.g., about 10 μg/mL, about 100 μg/mL, about 300 μg/mL, or any range between the recited values), preferably, the molar ratio of the human IgG to the rabbit IgG ranges from about 0.1:10 to about 10:0.1. In some preferred embodiments, the third blocking solution comprises a human IgG antibody at a concentration of about 10 μg/mL. In some preferred embodiments, the third blocking solution comprises a rabbit IgG antibody at a concentration of about 10 μg/mL. In some embodiments, the molar ratio of human IgG to rabbit IgG in the third blocking solution is about 1:1.

In some embodiments, the blocking step b) comprises treating the surface-agent-functionalized surface with a fourth blocking solution comprising a fragment crystallizable (Fc) region of an IgG antibody, such as a human, rabbit, or mouse IgG antibody, preferably, rabbit Fc region, for example, at a concentration of about 0.1 μg/mL to about 10 μg/mL, such as about 0.1 μg/mL, about 0.5 μg/mL, about 1 μg/mL, about 5 μg/mL, about 10 μg/mL, or any range between the recited values. In some embodiments, the fourth blocking solution comprises a rabbit IgG, such as the Fc region of a rabbit IgG, for example, at a concentration of about 0.5 μg/mL.

Typically, the first, second, third, and fourth blocking solutions are different from each other, and two or more of the first, second, third, and fourth blocking solutions are used to treat the surface-agent-functionalized surface sequentially in any order. For example, in some embodiments, the blocking step b) comprises treating the surface-agent-functionalized surface with the first blocking solution followed by the second blocking solution. In some embodiments, the blocking step b) comprises treating the surface-agent-functionalized surface with the first blocking solution followed by the third blocking solution and then the second blocking solution. In some embodiments, the blocking step b) does not include treating the surface-agent-functionalized surface with the third blocking solution. Typically, the fourth blocking solution is used to treat the surface-agent-functionalized surface after the second blocking solution, for example, during the analysis of biomarkers.

For example, in some embodiments, the blocking can be characterized as a “partial blocking”, which can include treating the surface:
(a) first with a modified polymer, preferably a PEG modified with amine, carboxyl, hydroxyl or thiol at a terminal end, preferably thiolated PEG, more preferably of an alkoxy having 1-20 carbon atoms such as methoxy modification at the other terminal end, more preferably thiolated methoxy PEG, preferably of molecular weight about 1000-5000 g/mol and concentration about 0.1-10 mM, preferably of molecular weight about 2000 g/mol and concentration about 2 mM (about 4 mg/mL), and then
(b) with a serum protein, such as albumin, fibrinogen, preferably albumin, more preferably bovine serum albumin of concentration about 0.1 to 5% (w/v), more preferably about 1%.

For example, in some embodiments, the blocking can be characterized as a “complete blocking”, which can include treating the surface:
(a) first with a modified polymer, preferably a PEG modified with amine, carboxyl, hydroxyl or thiol at a terminal end, preferably thiolated PEG, more preferably of an alkoxy having 1-20 carbon atoms such as methoxy modification at the other terminal end, more preferably thiolated methoxy PEG, preferably of molecular weight about 1000-5000 g/mol and concentration about 0.1-10 mM, preferably of molecular weight about 2000 g/mol and concentration about 2 mM (about 4 mg/mL), and then
(b) a mixture of human IgG and rabbit IgG: A human blocking agent, preferably human antibody, more preferably human IgG of concentration about 10-300 μg/mL, more preferably about 10 μg/mL. A rabbit blocking agent, preferably rabbit antibody, more preferably rabbit IgG of concentration about 10-300 μg/mL, more preferably about 10 μg/mL. The molar ratio of human IgG to rabbit IgG, preferably about 0.1:10 to about 10:0.1, more preferably about 1:1; and then
(c) with a serum protein, such as albumin. fibrinogen, preferably albumin, more preferably bovine serum albumin of concentration about 0.1 to 5% (w/v), more preferably about 1%

In some preferred embodiments, the blocking step b) can comprise treating the surface-agent-functionalized surface with a combined blocking solution comprising (i) a modified polyethylene glycol (PEG), wherein the modified PEG is modified with an amine, carboxyl, or thiol at one end; and (ii) a serum protein. Useful modified PEG and serum protein include any of those described herein. For example, in some embodiments, the modified PEG is a thiolated PEG, wherein the PEG is modified with a thiol at one end. In some embodiments, the thiolated PEG is capped with an alkoxy having 1-20 carbon atoms, preferably, methoxy, at the other end. In some specific embodiments, the thiolated PEG can be methoxypolyethylene glycol thiol characterized as having a general formula of CH₃O(CH₂CH₂O)ₙCH₂CH₂SH. In some preferred embodiments, the modified PEG (such as the thiolated PEG) has a number or weight average molecular weight, preferably, a number average molecular weight, of about 1000 g/mol to about 5000 g/mol, such as about 1000 g/mol, about 2000 g/mol, about 3000 g/mol, about 4000 g/mol, about 5000 g/mol, or any range between the recited values. In some embodiments, the modified PEG can have a number average molecular weight of about 2000 g/mol. In some embodiments, the serum protein is albumin and/or fibrinogen, preferably, albumin. In some embodiments, the serum protein is albumin, such as bovine serum albumin.

The combined blocking solution typically comprises the modified PEG at a concentration about 0.1 mM to about 10 mM and the serum protein at a concentration ranging from about 0.1% to about 5% (w/v), such as about 0.1%, about 1%, about 2%, about 5%, or any range between the recited values. In some specific embodiments, the combined blocking solution comprises the modified PEG at a concentration of about 2 mM and the serum protein at a concentration of about 1%. In some specific embodiments, the combined blocking solution comprises the modified PEG at a concentration of about 4 mg/ml and the serum protein at a concentration of about 1%.

In some embodiments, the combined blocking solution further comprises an antibody, preferably, the antibody is of the IgG isotype. For example, in some embodiments, the combined blocking solution further comprises a human antibody, a mouse antibody, and/or a rabbit antibody, for example, a mixture of human IgG and rabbit IgG. In some embodiments, the combined blocking solution comprises a human IgG antibody at a concentration of about 10-300 μg/mL (e.g., about 10 μg/mL, about 100 μg/mL, about 300 μg/mL, or any range between the recited values) and a rabbit IgG antibody at a concentration of about 10-300 μg/mL (e.g., about 10 μg/mL, about 100 μg/mL, about 300 μg/mL, or any range between the recited values), preferably, the molar ratio of the human IgG to the rabbit IgG ranges from about 0.1:10 to about 10:0.1. In some preferred embodiments, the combined blocking solution comprises a human IgG antibody at a concentration of about 10 μg/mL. In some preferred embodiments, the combined blocking solution comprises a rabbit IgG antibody at a concentration of about 10 μg/mL. In some embodiments, the molar ratio of human IgG to rabbit IgG in the combined blocking solution is about 1:1. In some embodiments, the combined blocking solution can also be free of the foregoing described antibody. In some embodiments, the combined blocking solution further comprises a fragment crystallizable (Fc) region of an IgG antibody, such as a human, rabbit, or mouse IgG antibody, preferably, rabbit Fc region, for example, at a concentration of about 0.1 μg/mL to about 10 μg/mL, such as about 0.1 μg/mL, about 0.5 μg/mL, about 1 μg/mL, about 5 μg/mL, about 10 μg/mL, or any range between the recited values. In some embodiments, the combined blocking solution comprises a rabbit IgG, such as the Fc region of a rabbit IgG, for example, at a concentration of about 0.5 μg/mL. However, in some embodiments, the combined blocking solution can also be free of the foregoing described Fc region of an IgG antibody.

Additional exemplified combined blocking solutions suitable for the blocking step b) are also described herein, such as in the Blocking Solution section.

The blocking step b) typically also includes treating the surface-agent-functionalized surface with one or more additional ingredients. For example, in some embodiments, the blocking step b) further comprising treating the surface-agent-functionalized surface with one or more ingredients selected from a buffer (e.g., phosphate buffered saline), a silane (e.g., decafluoro-1,1,2,2,-tetrahydrooctyl trichlorosilane (FOTS)), a surfactant (e.g., egg phosphatidylcholine, palmitoyl-oleoylphosphatidylcholine (POPC), Triton X, Tween 20, etc.), and a thiol (e.g., mercaptopropanol (MPO)).

Suitable buffer for use in the methods herein are not particularly limited. In some embodiments, the buffer can be phosphate-buffered saline (PBS), sodium chloride-sodium phosphate-EDTA, or HEPES (4-(2-hydroxyethyl)-1-piperazineethanesulfonic acid). Preferably, the pH of the buffer can range from about 6 to about 8, such as about 7.4. The concentration of the buffer is not particularly limited, which can be for example at about 0.1-10×PBS, more preferably about 2×PBS.

Suitable surfactants for use in the methods herein are also not particularly limited. For example, in some embodiments, the surfactant can be Tween 20, which can be at a concentration of about 0.001-1% (v/v), more preferably about 0.005% (v/v).

The methods of preparing surface herein can also optionally include treating the surface-agent-functionalized surface with a salt (e.g., sodium chloride) and/or a chelating agent (e.g., ethylenediaminetetraacetic acid (EDTA)). For example, in some embodiments, the surface-agent-functionalized surface can be treated with a salt, such as sodium chloride (NaCl), more preferably of concentration about 50 mM-5 M, more preferably about 274 mM. In some embodiments, the surface-agent-functionalized surface can be treated with a chelating agent, preferably ethylenediaminetetraacetic acid (EDTA), more preferably of concentration about 1-50 mM, more preferably about 20 mM.

### Blocking Solution

Some embodiments of the present disclosure are directed to blocking solutions and kits comprising blocking solution(s) herein.

In some embodiments, the present disclosure provides a combined blocking solution, which can be any of those described herein. As described herein, the combined blocking solution can include all of the desired blocking agent(s) in one solution, which can be convenient for use in blocking surfaces in some applications.

In some specific embodiments, the combined blocking solution comprises (a) a thiolated PEG, wherein the PEG is modified with a thiol at one end; (b) a serum protein; and optionally (c) an antibody, such as a human antibody, a mouse antibody, and/or a rabbit antibody. In some embodiments, the thiolated PEG is modified with a thiol at one end and is capped with an alkoxy having 1-20 carbon atoms such as a methoxy at the other end. For example, in some specific embodiments, the thiolated PEG can be methoxypolyethylene glycol thiol characterized as having a general formula of CH₃O(CH₂CH₂O)ₙCH₂CH₂SH. In some preferred embodiments, the thiolated PEG has a number or weight average molecular weight, preferably, a number average molecular weight, of about 1000 g/mol to about 5000 g/mol, such as about 1000 g/mol, about 2000 g/mol, about 3000 g/mol, about 4000 g/mol, about 5000 g/mol, or any range between the recited values. In some embodiments, the thiolated PEG can have a number average molecular weight of about 2000 g/mol. In some embodiments, the scrum protein is albumin and/or fibrinogen, preferably, albumin. In some embodiments, the serum protein is albumin, such as bovine serum albumin. In some embodiments, the combined blocking solution comprises (c) the antibody. For example, in some embodiments, the combined blocking solution comprises (a) the thiolated PEG; (b) the serum protein; and (c) a mixture of a human antibody and rabbit antibody. In some embodiments, the combined blocking solution can also be free of the optional antibody.

In some specific embodiments, the combined blocking solution comprises (a) methoxypolyethylene glycol thiol having a number or weight average molecular weight, preferably, a number average molecular weight, of about 1000 g/mol to about 5000 g/mol, such as about 1000 g/mol, about 2000 g/mol, about 3000 g/mol, about 4000 g/mol, about 5000 g/mol, or any range between the recited values; (b) the scrum protein; and optionally (c) the antibody, such as a mixture of a human antibody and rabbit antibody.

In some specific embodiments, the combined blocking solution comprises (a) the thiolated PEG; (b) bovine serum albumin; and optionally (c) the antibody, such as a mixture of a human antibody and rabbit antibody.

In some specific embodiments, the combined blocking solution comprises (a) the thiolated PEG; (b) albumin; and (c) a mixture of a human IgG and rabbit IgG, with a molar ratio of the human IgG to the rabbit IgG ranging from about 0.1:10 to 10:0.1, such as about 1:1.

In some specific embodiments, the combined blocking solution comprises (a) methoxypolyethylene glycol thiol having a number or weight average molecular weight, preferably, a number average molecular weight, of about 1000 g/mol to about 5000 g/mol, such as about 1000 g/mol, about 2000 g/mol, about 3000 g/mol, about 4000 g/mol, about 5000 g/mol, or any range between the recited values; (b) bovine serum albumin; and optionally (c) the antibody.

In some specific embodiments, the combined blocking solution comprises (a) methoxypolyethylene glycol thiol having a number or weight average molecular weight, preferably, a number average molecular weight, of about 1000 g/mol to about 5000 g/mol, such as about 1000 g/mol, about 2000 g/mol, about 3000 g/mol, about 4000 g/mol, about 5000 g/mol, or any range between the recited values; (b) bovine serum albumin; and (c) a mixture of a human antibody and rabbit antibody, preferably a mixture of human IgG and rabbit IgG.

In some specific embodiments, the combined blocking solution comprises (a) methoxypolyethylene glycol thiol having a number or weight average molecular weight, preferably, a number average molecular weight, of about 1000 g/mol to about 5000 g/mol, such as about 1000 g/mol, about 2000 g/mol, about 3000 g/mol, about 4000 g/mol, about 5000 g/mol, or any range between the recited values; (b) bovine serum albumin; and (c) a mixture of human IgG and rabbit IgG, with a molar ratio of the human IgG to the rabbit IgG ranging from about 0.1:10 to 10:0.1, such as about 1:1.

In some embodiments, the combined blocking solution further comprises a fragment crystallizable (Fc) region of an IgG antibody, such as a human, rabbit, or mouse IgG antibody, preferably, rabbit Fc region, for example, at a concentration of about 0.1 μg/mL to 10 μg/mL.

The combined blocking solution typically comprises the thiolated PEG at a concentration about 0.1 mM to about 10 mM and the serum protein at a concentration ranging from about 0.1% to about 5% (w/v), such as about 0.1%, about 1%, about 2%, about 5%, or any range between the recited values. In some specific embodiments, the combined blocking solution comprises the thiolated PEG at a concentration of about 2 mM and the serum protein at a concentration of about 1% (w/v). In some specific embodiments, the combined blocking solution comprises the thiolated PEG at a concentration of about 4 mg/ml and the serum protein at a concentration of about 1% (w/v).

In some embodiments, the combined blocking solution comprises a mixture of human IgG and rabbit IgG. In such embodiments, the combined blocking solution can typically comprise the human IgG antibody at a concentration of about 10-300 μg/mL (e.g., about 10 μg/mL, about 100 μg/mL, about 300 μg/mL, or any range between the recited values) and the rabbit IgG antibody at a concentration of about 10-300 μg/mL (e.g., about 10 μg/mL, about 100 μg/mL, about 300 μg/mL, or any range between the recited values), preferably, the molar ratio of the human IgG to the rabbit IgG ranges from about 0.1:10 to 10:0.1. In some preferred embodiments, the combined blocking solution comprises the human IgG antibody at a concentration of about 10 μg/mL. In some preferred embodiments, the combined blocking solution comprises the rabbit IgG antibody at a concentration of about 10 μg/mL. In some embodiments, the molar ratio of the human IgG to rabbit IgG in the combined blocking solution is about 1:1.

In some embodiments, the combined blocking solution can comprise: (a) methoxypolyethylene glycol thiol herein, preferably having a number average molecular weight of about 2000 g/mol, in a concentration of about 0.1 mM to about 10 mM, preferably, about 2 mM; and (b) bovine serum albumin, preferably in a concentration of about 0.1% to about 5% (w/v), preferably, about 1% (w/v).

In some embodiments, the combined blocking solution can comprise: (a) methoxypolyethylene glycol thiol herein, preferably having a number average molecular weight of about 2000 g/mol, in a concentration of about 0.1 mM to about 10 mM, preferably, about 2 mM; (b) bovine serum albumin, preferably in a concentration of about 0.1% to about 5% (w/v), preferably, about 1% (w/v); and (c) a mixture of human IgG and rabbit IgG, preferably, the human IgG is at a concentration of about 10-300 μg/mL, more preferably about 10 μg/mL, and the rabbit IgG is at a concentration of about 10-300 μg/mL, more preferably about 10 μg/mL, wherein the molar ratio of the human IgG to the rabbit IgG ranging from about 0.1:10 to 10:0.1, preferably at about 1:1.

In some embodiments, the combined blocking solution further comprises a fragment crystallizable (Fc) region of an IgG antibody, such as a human, rabbit, or mouse IgG antibody, preferably, rabbit Fc region, for example, at a concentration of about 0.1 μg/mL to about 10 μg/mL, such as about 0.1 μg/mL, about 0.5 μg/mL, about 1 μg/mL, about 5 μg/mL, about 10 μg/mL, or any range between the recited values. In some embodiments, the combined blocking solution comprises a rabbit IgG, such as the Fc region of a rabbit IgG, for example, at a concentration of about 0.5 μg/mL. However, in some embodiments, the combined blocking solution can also be free of the foregoing described Fc region of an IgG antibody.

In some embodiments, the combined blocking solution can comprise about 4 mg/mL thiolated polyethylene glycol (PEG-SH), about 10 μg/mL IgG mix (consists of rabbit IgG, human IgG, and/or mouse IgG), about 1% (w/v) bovine serum albumin (BSA), and about 0.1-0.5 μg/mL Fc fragment in about 1-2×phosphate-buffered saline (PBS) solution.

In some embodiments, the combined blocking solution can comprise about 4 mg/ml thiolated methoxy polyethylene glycol (mPEG-SH, MW of 2k), IgG mix (1:1 molar ratio rabbit IgG and human IgG, about 10 μg/mL each), about 1% (w/v) bovine serum albumin (BSA) and about 0.5 μg/mL Fc fragment in 1× phosphate-buffered saline (PBS) solution.

In some embodiments, the combined blocking solution can further comprise one or more optional ingredients, such as a buffer. For example, in some embodiments, the combined blocking solution can comprise phosphate-buffered saline (PBS), sodium chloride-sodium phosphate-EDTA, or HEPES (4-(2-hydroxyethyl)-1-piperazinecthanesulfonic acid), at a pH of about 6-8.

In some embodiments, all of the blocking agents are not included in one solution. In such embodiments, a combination of blocking agents is typically provided in two or more different blocking solutions as described herein.

For example, in some embodiments, the present disclosure provides a combination of blocking agents comprising (a) a first solution comprising a thiolated PEG, wherein the PEG is modified with a thiol at one end; (b) a second solution comprising a serum protein; and optionally (c) a third solution comprising an antibody, such as a human antibody, a mouse antibody, and/or a rabbit antibody, wherein the first, second, and third solution do not contain the same blocking agent(s).

In some embodiments, the thiolated PEG in the first solution is modified with a thiol at one end and is capped with an alkoxy having 1-20 carbon atoms such as a methoxy at the other end. For example, in some specific embodiments, the thiolated PEG can be methoxypolyethylene glycol thiol characterized as having a general formula of CH₃O(CH₂CH₂O)ₙCH₂CH₂SH. In some preferred embodiments, the thiolated PEG has a number or weight average molecular weight, preferably, a number average molecular weight, of about 1000 g/mol to about 5000 g/mol, such as about 1000 g/mol, about 2000 g/mol, about 3000 g/mol, about 4000 g/mol, about 5000 g/mol, or any range between the recited values. In some embodiments, the thiolated PEG can have a number average molecular weight of about 2000 g/mol. In some embodiments, the first solution can comprise a methoxypolyethylene glycol thiol having a number or weight average molecular weight, preferably, a number average molecular weight, of about 1000 g/mol to about 5000 g/mol, such as about 1000 g/mol, about 2000 g/mol, about 3000 g/mol, about 4000 g/mol, about 5000 g/mol, or any range between the recited values, for example, at a concentration about 0.1 mM to about 10 mM. In some embodiments, the first solution can comprise a methoxypolyethylene glycol thiol having a number average molecular weight of about 2000 g/mol, in a concentration of about 0.1 mM to about 10 mM, preferably, about 2 mM.

In some embodiments, the serum protein in the second solution is albumin and/or fibrinogen, preferably, albumin. In some embodiments, the serum protein is albumin, such as bovine serum albumin. In some embodiments, the second solution can comprise the serum protein at a concentration ranging from about 0.1% to about 5% (w/v), such as about 0.1%, about 1%, about 2%, about 5%, or any range between the recited values. For example, in some embodiments, the second solution can comprise bovine serum albumin, which for example can be at a concentration ranging from about 0.1% to about 5% (w/v), such as about 1% (w/v).

In some embodiments, the combination of blocking agents comprises the third solution. In some embodiments, the antibody in the third solution can be a mixture of human IgG and rabbit IgG. For example, in some embodiments, the third solution can comprise the human IgG antibody at a concentration of about 10-300 μg/mL (e.g., about 10 μg/mL, about 100 μg/mL, about 300 μg/mL, or any range between the recited values) and the rabbit IgG antibody at a concentration of about 10-300 μg/mL (e.g., about 10 μg/mL, about 100 μg/mL, about 300 μg/mL, or any range between the recited values), preferably, the molar ratio of the human IgG to the rabbit IgG ranges from about 0.1:10 to 10:0.1. In some preferred embodiments, the third solution comprises the human IgG antibody at a concentration of about 10 μg/mL. In some preferred embodiments, the third solution comprises the rabbit IgG antibody at a concentration of about 10 μg/mL. In some embodiments, the molar ratio of the human IgG to rabbit IgG in the third solution is about 1:1. In some embodiments, the combination of blocking agents does not include the third solution.

In some embodiments, the combination of blocking agents can further comprise a fourth solution comprising a fragment crystallizable (Fc) region of an IgG antibody, such as a human, rabbit, or mouse IgG antibody, preferably, rabbit Fc region, for example, at a concentration of about 0.1 μg/mL to about 10 μg/mL, such as about 0.1 μg/mL, about 0.5 μg/mL, about 1 μg/mL, about 5 μg/mL, about 10 μg/mL, or any range between the recited values. In some embodiments, the fourth solution comprises a rabbit IgG, such as the Fc region of a rabbit IgG, for example, at a concentration of about 0.5 μg/mL. However, in some embodiments, the combined blocking solution does not include the fourth solution.

The first, second, third, and fourth solution typically also comprise a buffer, such as phosphate-buffered saline (PBS), sodium chloride-sodium phosphate-EDTA, or HEPES (4-(2-hydroxyethyl)-1-piperazineethanesulfonic acid), at a pH of about 6-8, such as about 7.4.

In some embodiments, the combination of blocking agents together can include about 4 mg/mL thiolated polyethylene glycol (PEG-SH), about 10 μg/mL IgG mix (consists of rabbit IgG, human IgG, and/or mouse IgG), about 1% (w/v) bovine serum albumin (BSA), and about 0.1-0.5 μg/mL Fc fragment in about 1-2×phosphate-buffered saline (PBS) solution, wherein the thiolated polyethylene glycol, IgG mix, BSA, and Fc fragment are not all in one solution, for example, each of the thiolated polyethylene glycol. IgG mix, BSA, and Fc fragment is included in a separate solution.

In some embodiments, the combination of blocking agents together can include about 4 mg/mL thiolated methoxy polyethylene glycol (mPEG-SH, MW of 2k), IgG mix (1:1 molar ratio rabbit IgG and human IgG, about 10 μg/mL each), about 1% (w/v) bovine serum albumin (BSA) and about 0.5 μg/mL Fc fragment in 1× phosphate-buffered saline (PBS) solution, wherein the mPEG-SH, IgG mix, BSA, and Fc fragment are not all in one solution, for example, each of the mPEG-SH, IgG mix, BSA, and Fc fragment is included in a separate solution.

It should also be clear that the combined blocking solution or the combination of blocking agents described herein can be used for the blocking step in any of the methods described herein for treating a surface described herein. And in the case of treating with the combination of blocking agents described herein, the treatment of the surface typically is conducted sequentially, such as with the first solution, followed by the second solution, or with the first solution, followed by the third solution and then the second solution, etc.

The surface (e.g., a gold surface described herein) or substrate comprising a surface (e.g., inert metal surface such as gold surface) treated with the combined blocking solution or the combination of blocking agents described herein are also novel embodiments of the present disclosure. For example, in some embodiments, the substrate comprises an inert metal surface, wherein the inert surface is treated with any of the combined blocking solution or the combination of blocking agents described herein. In some embodiments, the inert metal surface is a gold surface, a silver surface, or a gold/silver alloy surface coated on the substrate. In some preferred embodiments, the substrate can be a glass substrate having a gold surface, wherein the gold surface is treated with any of the combined blocking solution or the combination of blocking agents described herein. Preferably, the substrate is suitable for use in a SPR analysis.

### Kits

In some embodiments, the surfaces/substrates, surface agents, and/or blocking agents or blocking solutions, can be included in a kit. For example, in some embodiments, the kit can include any of the combination of blocking agents and a container. In some embodiments, the kit can include any of the combined blocking solutions and a container.

In some specific embodiments, the present disclosure provides a kit comprising (i) a substrate having an inert metal surface; and (ii) any of the combined blocking solution as described herein. In some embodiments, the present disclosure provides a kit comprising (i) a substrate having an inert metal surface; and (ii) any of the combination of blocking agents as described herein. Typically, the inert metal surface is a gold, silver, or gold/silver alloy surface coated on the substrate. The substrate can be any of those described herein as suitable, such as a glass, metal, ceramic, and/or polymer substrate, preferably a glass substrate. For example, in some preferred embodiments, the substrate can be a glass substrate having a gold surface, which is suitable for use in a SPR analysis.

In some embodiments, the kit can further comprise a surface agent described herein. For example, in some embodiments, the kit can comprise the substrate having the inert metal surface, wherein the inert metal surface is functionalized with the surface agent described herein. In some embodiments, the kit can comprise (i) the substrate having the inert metal surface, (ii) any of the combined blocking solution or the combination of blocking agents as described herein, and (iii) the surface agent, wherein the inert metal surface is not functionalized with the surface agent described herein. The surface agent is not particularly limited and include any of those described herein. For example, in some preferred embodiments, the surface agent is a thiolated protein A, wherein the protein A is modified with a thiol.

In some embodiments, the kit can further comprise a surface-agent-dependent capture molecule, wherein the surface agent is capable of binding to the inert metal surface and specifically binding to the surface-agent-dependent capture molecule, wherein the surface-agent-dependent capture molecule is capable of specifically binding to one or more analytes. For example, in some embodiments, the inert metal surface can comprise the surface agent and surface-agent-dependent capture molecule, wherein the surface agent bound to the inert metal surface specifically binds to the surface-agent-dependent capture molecule, wherein the surface-agent-dependent capture molecule is capable of specifically binding to one or more analytes.

In some embodiments, the kit can further comprise a surface-agent-independent capture molecule, wherein the surface-agent-independent capture molecule is capable of directly or indirectly binding to the inert surface without binding to a surface agent, and the surface-agent-independent capture molecule is capable of specifically binding to one or more analytes. For example, in some embodiments, the inert metal surface can comprise the surface agent and surface-agent-independent capture molecule, wherein the surface agent bound to the inert metal surface, and the surface-agent-independent capture molecule is capable of directly or indirectly binding to the inert metal surface without binding to the surface agent, and the surface-agent-independent capture molecule is capable of specifically binding to one or more analytes.

Suitable surface-agent-dependent and surface-agent-independent capture molecules are not particularly limited and include any of those described herein.

In some embodiments, the kit can include one or more ingredients selected from a buffer (e.g., phosphate buffered saline), a silane (e.g., decafluoro-1,1,2,2,-tetrahydrooctyl trichlorosilane (FOTS)), a surfactant (e.g., egg phosphatidylcholine, palmitoyl-oleoylphosphatidylcholine (POPC), Triton X. Tween 20, etc.), and a thiol (e.g., mercaptopropanol (MPO)).

In some embodiments, the kit can include a buffer, such as phosphate-buffered saline (PBS), sodium chloride-sodium phosphate-EDTA, or HEPES (4-(2-hydroxyethyl)-1-piperazineethanesulfonic acid), preferably, the buffer has a pH of about 6-8. The concentration of the buffer is not particularly limited, which can be for example at about 0.1-10×PBS, more preferably about 2×PBS.

In some embodiments, the kit can include a surfactant, such as Tween 20, which can be at a concentration of about 0.001-1% (v/v), more preferably about 0.005% (v/v).

In some embodiments, the kit can include a salt (e.g., sodium chloride) such as sodium chloride (NaCl), more preferably of concentration about 50 mM-5M, more preferably about 274 mM.

In some embodiments, the kit can include a chelating agent (e.g., ethylenediaminetetraacetic acid (EDTA)), more preferably of concentration about 1-50 mM, more preferably about 20 mM.

In some embodiments, the kit can include one or more components selected from antigens, serum samples, inhibitors that stabilize a pathogen-related DNA or RNA target in the serum samples, detection molecules such as detection aptamers and detection antibodies, and nanoenhancers.

In some embodiments, the kit can include an antigen, such as those specifically bind with antibodies against infectious diseases, such as Zika, Dengue, and/or Chikungunya viruses, those that are related to liver, kidney, brain, and/or lung diseases, those that are cytokines related to atopic dermatitis, those that are exosomes associated with pancreatic cancer, etc. Concentrations of such antigens are not particularly limited. For example, when the agent is to used in conjunction with a microspotter, such as to be spotted onto a gold film of a sensing chip using a microspotter, the antigen can have a concentration preferably in the range of about 5 μg/mL to about 100 μg/mL, more preferably about 2.5 μg/mL or about 25 μg/mL. In some embodiments, the antigen can be added during an analysis of the biosamples or can be spotted on top of a capture antibody on the surface, which can have a concentration of about 50 g/mL to about 500 μg/mL, more preferably about 350 μg/mL.

In some embodiments, the kit can include a serum sample to be analyzed. For example, the kit can include a human serum sample to be used for analysis, preferable of concentration about 1-100%, more preferably about 10%.

In some embodiments, the kit can include an inhibitor, preferably used to stabilize the pathogen-related DNA or RNA target in the serum sample, preferably enzyme, preferably RNAse inhibitor, preferably of concentration about 40 U/μL, preferably of volume about 0 μL-5 μL, more preferably about 1 μL, more preferably added in the serum sample and injected during the analysis of biomarkers.

In some embodiments, the kit can include a detection aptamer, preferably nucleic acids specific to detect pathogen, organ injury, and/or skin disease targets, preferably targets associated with Zika, Dengue, and/or Chikungunya viruses, or liver, kidney, brain, or lung injuries, more preferably DNA detection aptamers, preferably modified detection aptamers, preferably of biotin, amine, carboxyl, hydroxyl and/or thiol modifications, more preferably of biotin conjugation, preferably of 15-90 nucleotides long, more preferably 30 nucleotides, preferably of concentrations about 10 nM-2 mM, such as about 10 nM, about 50 nM, about 100 nM, about 200 nM, about 300 nM, about 500 nM, about 1 mM, about 2 mM, or any range between the recited values, more preferably about 50 nM. When modified, the detection aptamer can be modified at the 5′ and/or 3′ ends, more preferably, at the 3′ end. The detection aptamer is typically for injection during the analysis of biomarkers.

In some embodiments, the detection aptamer can be selected from nucleic acids with or without structure modifications. In some preferred embodiments, the detection aptamer can have structure modifications, such as locked nucleic acids and peptide nucleic acids, more preferably locked nucleic acids. When the structures of the nucleic acids are modified, the modification is typically for increasing melting temperature and/or strengthening the nucleic acids' binding with targets such as mRNA, DNA, RNA of shorter sequences, preferably mRNA of 18nt-60 nt long. In some preferred embodiments, the detection aptamer can be selected from single-stranded and double-stranded aptamers, more preferably single-stranded aptamers, preferably 5-60 nucleotides long, more preferably 12 nt long. The concentrations of the detection aptamer for use herein can preferably be in the range of 1 nM-2 mM, such as about 1 nM, about 5 nM, about 10 nM, about 50 nM, about 100 nM, about 500 nM, about 1 mM, about 2 mM, or any range between the recited values, such as about 10 nM. In some embodiments, the detection aptamer can be injected during the analysis of a biomarker.

In some embodiments, the kit can include a primary detection antibody (primary antibody), such as primary antibodies against Zika, Dengue, and/or Chikungunya viruses or antigens, or liver, kidney, brain, lung, or post-traumatic stress disorder (PTSD) biomarkers, preferably, the detection antibody is an IgG, preferably modified with biotin, streptavidin, amine, hydroxyl, carboxyl, and/or thiol, preferably biotin, preferably of concentration about 1 μg/mL-50 μg/mL, more preferably about 2.5 μg/mL. The primary detection antibody is typically injected during the analysis of biomarkers.

In some embodiments, the kit can include a secondary detection antibody (secondary antibody), such as secondary antibodies against Zika, Dengue, and/or Chikungunya antibodies, preferably secondary antibodies are against the Fc region of the unconjugated (i.e., no biotin) primary antibody IgM, IgG, IgD, IgA, or IgE, preferably against IgG or IgM, more preferably anti-IgG and/or anti-IgM, preferably of goat, rabbit, and mouse hosts, preferably goat anti-IgG and/or goat anti-IgM, preferably goat anti-human IgM, and goat anti-human IgG, preferably modified with biotin, streptavidin, amine, hydroxyl, carboxyl, and/or thiol, preferably biotin, preferably of concentration about 1 μg/mL-50 μg/mL, more preferably about 2.5 μg/mL and about 10 μg/mL. The secondary detection antibody is typically injected during the analysis of biomarkers.

In some embodiments, the kit can include a nanoenhancer, preferably quantum dots, semiconductor nanoparticles, and/or noble metal nanoparticles, preferably quantum dots, preferably of near-infrared emission range, more preferably of 800 nm emission maxima, preferably less than 100 nm size, more preferably about 20 nm, preferably modified with streptavidin, preferably of concentrations about 1-30 mM, more preferably about 10 nM.

In some embodiments, the kit can include a further blocking agent to block the active site of a streptavidin-coated nanoenhacer, preferably biotin, streptavidin, amine, hydroxyl, carboxyl, and/or thiol, preferably biotin, preferably of concentration about 0.5-100 M, more preferably about 3 μM. The blocking agent is preferably injected during the analysis of biomarkers.

In some embodiments, the kit can also include a press load as described herein.

In some embodiments, the kit can also include a microspotter, such as a CFM, as described herein.

In some embodiments, the kit can include a container.

For components, such as certain blocking agents, that can be provided as a solution or in another form, such as a powder, a solid or liquid, the kit can include such components in a solution form or any other form. For example, for any of the embodiments according to a kit comprising a combined blocking solution herein, alternative embodiments are also provided for a kit comprising the blocking agents in the combined blocking solution in any other forms. Similarly, for any of the embodiments according to a kit comprising a combination of blocking agents in solution forms herein, alternative embodiments are also provided for a kit comprising the blocking agents of the combination of blocking agents in any other forms.

Typically, the kit can also include instructions on surface functionalization, array construction, and/or surface blocking. In some embodiments, the kit can also include any hardware, such as a computer, that may be used in connection with an analysis of a sample using the substrate. In some embodiments, the kit can also include a software useful for an analysis of a sample using the substrate, such as for array construction and/or data analysis.

### Substrates

Some embodiments of the present disclosure are directed to substrates having a functionalized, printed, and/or blocked surface herein.

In some embodiments, the present disclosure provides a substrate having an inert metal surface, wherein the inert metal surface comprises:
a) a surface-agent-dependent capture molecule, which is immobilized on the inert metal surface through specific binding to a surface agent bound to the inert metal surface; and
b) a plurality of blocking agents, which are bound to the inert metal surface directly or indirectly,
wherein the surface-agent-dependent capture molecule is capable of specifically binding to one or more analytes, wherein the plurality of blocking agents is capable of reducing (preferably substantially reducing) or preventing the inert metal surface from non-specific binding.

In some embodiments, the substrate can be a glass, metal, ceramic, and/or polymer substrate, preferably a glass substrate. Typically, the inert metal surface is a gold surface coated on the substrate. For example, in some preferred embodiments, the substrate can be a glass substrate having a gold surface, which is suitable for use in a SPR analysis. In some embodiments, the inert metal surface can also be a silver surface or a gold/silver alloy surface.

In some embodiments, the surface agent can be uniformly bound to the inert metal surface. For example, such substrate can be prepared by using a press load as described herein.

In some embodiments, the surface agent can be bound to the inert metal surface at a predefined area. For example, the surface agent can be spotted at different spots of the inert metal surface, such as using a microspotter as described herein.

The surface agent is typically covalently bound to the inert metal surface, for example, through a functional group such as an amine, carboxyl, hydroxyl, and/or thiol. In preferred embodiments, the surface agent can be a protein, preferably, protein A, protein G, protein L, avidin, or streptavidin. For example, in some preferred embodiments, the surface agent can be protein A modified with amine, carboxyl, hydroxyl, and/or thiol. In any of the embodiments described herein, unless otherwise specified or contrary from context, the surface agent can be thiolated protein A, wherein the protein A is modified with a thiol.

The surface-agent-dependent capture molecule is typically a capture antibody. For example, in some embodiments, the surface agent can be thiolated protein A and the surface-agent-dependent capture molecule is an IgG antibody. In some embodiments, the capture antibody can be selected from antibodies against diseases such as infectious diseases, organ injuries, and/or skin diseases, preferably antibodies against Zika, Dengue, and/or Chikungunya viruses, liver, kidney, brain, or lung biomarkers, cytokines related to atopic dermatitis and/or exosomes associated with pancreatic cancer, etc. In some embodiments, the capture antibody can be selected from antibodies of different isotypes IgM, IgG, IgD, IgA, or IgE, preferably of IgG isotype, preferably IgG with Fc region that can bind with protein A to afford an upright antibody orientation.

In some embodiments, the surface-agent-dependent capture molecule can also include any of those that can specifically bind to the capture antibody described herein. For example, in some embodiments, the surface-agent-dependent capture molecule can include an antigen, such as those specifically bind with antibodies against infectious diseases, such as Zika, Dengue, and/or Chikungunya viruses, those that are related to liver, kidney, brain, and/or lung diseases, those that are cytokines related to atopic dermatitis, those that are exosomes associated with pancreatic cancer.

In some embodiments, the substrate can further comprise a surface-agent-independent capture molecule, directly or indirectly bound to the inert metal surface without binding to the surface agent. For example, in some embodiments, the surface-agent-independent capture molecule can be a capture aptamer, preferably nucleic acids specific to biomarker targets associated with pathogens, organ injuries, skin diseases, and/or psychiatric disorder, preferably capture aptamers specific to DNA/RNA targets associated with Zika, Dengue, and/or Chikungunya viruses, or liver, kidney, brain, lung, or post-traumatic stress disorder (PTSD) biomarkers, more preferably DNA aptamers.

The plurality of blocking agents of the substrate typically comprise a modified polyethylene glycol (PEG), wherein the modified PEG is modified with an amine, carboxyl, or thiol at one end. Preferably, the modified PEG is a thiolated PEG, wherein the PEG is modified with a thiol at one end. In some embodiments, the thiolated PEG is capped with an alkoxy having 1-20 carbon atoms such as a methoxy at the other end. In some specific embodiments, the thiolated PEG can be methoxypolyethylene glycol thiol characterized as having a general formula of CH₃O(CH₂CH₂O)ₙCH₂CH₂SH. The molecular weight of the modified PEG is not particularly limited. However, in some preferred embodiments, the modified PEG (such as the thiolated PEG) has a number or weight average molecular weight, preferably, a number average molecular weight, of about 1000 g/mol to about 5000 g/mol, such as about 1000 g/mol, about 2000 g/mol, about 3000 g/mol, about 4000 g/mol, about 5000 g/mol, or any range between the recited values. In some embodiments, the modified PEG can have a number average molecular weight of about 2000 g/mol.

In some embodiments, the plurality of blocking agents further comprise a serum protein. In some embodiments, the serum protein is albumin and/or fibrinogen, preferably, albumin. In some embodiments, the serum protein is albumin, such as bovine serum albumin.

In some embodiments, the plurality of blocking agents further comprise an antibody, such as a human antibody, a mouse antibody, and/or a rabbit antibody, preferably, the antibody is of the IgG isotype. For example, in some embodiments, the antibody comprises a mixture of human IgG and rabbit IgG. Preferably, the molar ratio of the human IgG to the rabbit IgG ranges from about 0.1:10 to 10:0.1. In some preferred embodiments, the molar ratio of human IgG to rabbit IgG in the third blocking solution is about 1:1.

In some embodiments, the plurality of blocking agents further comprise a fragment crystallizable (Fc) region of an IgG antibody, such as a human, rabbit, or mouse IgG antibody, preferably, rabbit Fc region.

The plurality of blocking agents can include further ingredients described herein, such as one or more ingredients selected from a buffer (e.g., phosphate buffered saline), a silane (e.g., decafluoro-1,1,2,2,-tetrahydrooctyl trichlorosilane (FOTS)), a surfactant (e.g., egg phosphatidylcholine, palmitoyl-oleoylphosphatidylcholine (POPC), Triton X, Tween 20, etc.), and a thiol (e.g., mercaptopropanol (MPO)).

In some embodiments, the substrate can include one or more components selected from antigens, serum samples, inhibitors that stabilize a pathogen-related DNA or RNA target in the serum samples, detection aptamers, detection antibodies, and nanoenhancers, which are bound to or otherwise in contact with the inert metal surface directly or indirectly. Suitable antigens, serum samples, inhibitors, detection aptamers, detection antibodies, and nanoenhances include any of those described herein.

As described herein, the surface treatment, surface, substrates, etc. described herein are subject to many different applications, such as for analyzing a sample, typically biological sample, such as a human serum sample.

For example, in some embodiments, the present disclosure provides a method of analyzing a sample, comprising (a) providing the substrate of any of those described herein, wherein the substrate comprises at least one capture molecule on the inert metal surface that is capable of specifically binding to an analyte; (b) incubating the sample with the substrate under a condition suitable for the at least one capture molecule to specifically bind to the analyte; and (c) determining whether the sample specifically binds the substrate, thereby determining whether the analyte is present in the sample. In some specific embodiments, the substrate is suitable for use in a surface plasmon resonance analysis. In some embodiments, the method comprises surface plasmon resonance analysis. For example, in some embodiments, the determining step c) comprises comparing surface plasmon resonance reflectivity of the substrate incubated with the sample or a control.

### Exemplified Applications in SPRi

In some embodiments, the surface treatment, surface, substrates, etc. described herein can be used on a SPRi-based system, for example, for target biomacromolecule detection in biological samples such as human serum samples.

A typical SPRi biosensor is a glass chip coated with a thin layer of chemically inert metal, usually gold. This chip is normally functionalized by an additional chemical coating, called the immobilization matrix. Ligands or target molecules are attached to the immobilization matrix via either chemical (or covalent) coupling, where a permanent covalent bond is formed, or via capture coupling (or affinity capture), where a non-covalent bond is made. Table 1 provides a list of existing biosensor chip surface chemistries and their applications (8).

Covalent coupling of the ligand (or target) is carried out using amine, thiol, aldehyde, carboxyl, or maleimide groups. Chips coated with carboxylated matrices, such as carboxymethyl dextran, are also widely used for immobilizing ligands ranging from carbohydrates to proteins at high densities. Capture coupling of ligand is based on their specific affinity to interaction partners. Examples are the interactions of biotin/avidin, Ni/NTA (nitrilotriacetic acid), antigen/antibody and protein A/IgG.

The advantages and disadvantages of various functionalization techniques under these two approaches are summarized in Table 2. Covalent coupling involves forming a permanent covalent bond of the ligand to the sensor chip surface. An advantage of covalent coupling, particularly amine coupling, is that it is straightforward, and multiple points of attachment are possible. Therefore, high density surfaces can be prepared. Because a permanent bond is made, the surface is stable. However, ligand orientation cannot be controlled and may result in low or no binding of the analyte. Moreover, the ligand may be deactivated or denatured either during coupling or due to the use of regeneration solutions between analyte injections.

TABLE 1
Summary of biosensor chip surface
chemistries and their applications.

| SPR chip surface chemistry | Applications |
|---|---|
| Long chain dextran/ | General purpose; suitable for protein- |
| carboxymethyl dextran/ | protein interactions and small |
| alginate | molecule analytes |
| Short chain dextran/ | Suitable for protein-protein interactions |
| carboxymethyl dextran/ | and for large analyte molecules |
| alginate or planar SAMs | and particles |
| Immobilized streptavidin | Capture of biotinylated ligands |
| Immobilized NTA | Capture of poly-histidine tagged ligands |
| Lipophilic modification | Capture of liposomes and supported lipid |
| | bilayers |
| Hydrophobic surface | Capture of lipid monolayers |
| Plain gold surface | Surface interaction studies and custom |
| | design of surface chemistry |
| Immobilized Protein A | Capture of IgG |

TABLE 2
Advantages and disadvantages of methods for immobilizing ligands on the biosensor chip surface.

| Sensor chip surface | Advantage | Disadvantage |
|---|---|---|
| A. Covalent | Stable surface. | Irreversible nature of ligand binding |
| coupling | Lower ligand consumption. | renders chip unusable if ligand is |
| | | denatured or inactivated. |
| | | Random orientation of ligand, which |
| | | can result in binding pocket being |
| | | inaccessible. |
| 1. Amine-carboxyl | Easy to perform and consistent. | Amine coupling not suitable for |
| or aldehyde based | Most macromolecules contain amine | acidic ligands ( [symbol] <3.5). |
| | groups: therefore, amine coupling is | |
| | the preferred method. | |
| 2. Disulfide based | Binding is more specific. | Some ligands don't have available |
| | Ligand binding can be reversed using | thiol groups (although they an be added). |
| | reducing agents. | Ligand consumption can be high. |
| B. Affinity capture | Ligand is specifically captured on chip. | Requires the added step of capture |
| based | Does not require highly purified ligand. | molecule immobilization. |
| | In many cases, no permanent bond to the | Ligand consumption high. |
| | surface is made and the chip can be | In many instances, ligand needs to |
| | regenerated. | be tagged. |
| 1. Biotin-avidin | Given the very high affinity of biotin- | Requires a conjugated (e.g., |
| based | avidin binding, ligand capture results in a | biotinylated) target. |
| | stable surface. | |
| 2. Ni-NTA based | Convenient for the capture of ligands | Can result in a decaying surface. |
| | carrying poly-histidine purification tags. | |
| | Chip is easily regenerated using imidazole | |
| | and/or EDTA. | |
| 3. Antibody based | Covalent coupling of antibodies provides | Only works for protein targets. |
| | a stable reusable chip. | |
| 4. Protein A-IgG | IgG ligand is easily eluted using a number | |
| based | of methods, which include low pH, high | |
| | pH, high ionic strength, and competitive | |
| | binding, for chip regeneration. This is | |
| | used as a functionalization agent in this | |
| | invention disclosure. | |

[symbol]

indicates data missing or illegible when filed

In the case of affinity capture, there is no permanent bond to the surface, and the chip can be regenerated. For protein A/IgG capture, the ligand is easily eluted using several methods (e.g., low pH, high pH, high ionic strength, and competitive binding) for chip regeneration.

The present disclosure typically uses protein A as a functionalization agent for immobilizing IgG capture antibody on the SPRi metal surface. The functionalization strategy used herein may employ a press load or a MS (multiple spotting) technology as discussed herein.

Microarray Printing Approaches: Microarrays are a commonly used tool in research and diagnostics. They come in a variety of forms such as antibody arrays, antigen arrays, DNA arrays, and bead arrays. They are used for efficient, high throughput testing of biofluids and patient samples.

Microarrays consist of hundreds or thousands of spots arranged in a well-defined fashion on a suitable surface. Table 3 summarizes the various array technologies to construct these microarrays. These surfaces can be glass slides (nitrocellulose-, polymer-, or N-hydroxysuccinimide-coated), nitrocellulose membrane, nitrocellulose filters, gel pads (acrylamide or gelatin), metal electrodes, epoxy-silane-derivatized or Teflon-masked slides, agarose film, and many others. Depending on the type of assay the array is used for, the spots can consist of various materials requiring adaptability of the dispensing or printing system which can be automated or robotic. Exemplary MS (multiple spotting) technology using HORIBA Scientific CFM is also included in the list to distinguish the novelty of the spotting approach used.

TABLE 3
Different methods to construct antibody, antigen, and DNA microarrays.

| | | | | |
|---|---|---|---|---|
| | Array | Printing | | |
| Name | Technology | Type | Printing Description | Reference |

Antibody microarray

| | | | | |
|---|---|---|---|---|
| Full Moon | Phospho | Single spot | Antibodies are covalently | https://www. [symbol] |
| Biosystems | Explorer S1 | | immobilized on high quality glass | [symbol] |
| | Antibody Array | | surface coated with proprietary 3-D | [symbol] |
| | | | polymer materials to ensure high | |
| | | | binding efficiency and specificity. | |
| Hypromatrix | Signal | Single spot | The antibodies are immobilized on a | https://www. [symbol] |
| | Transduction | | membrane, each at a predetermined | [symbol] |
| | Antibody Array | | position, and they retain their | [symbol] |
| | | | capabilities of recognizing and | |
| | | | capturing antigens as well as | |
| | | | antigen-associated proteins. The | |
| | | | proteins captured on the array can | |
| | | | then be detected by immunoblotting. | |
| Antigen microarray | RayBio Antibody | Single spot | A panel of capture antibodies is | https://www. [symbol] |
| RayBiotech, Inc. | Array G Series | | printed in multiple identical arrays | [symbol] |
| | | | on a standard slide. | [symbol] |
| R&D Systems | Proteome | Single spot | Capture antibodies are spotted on | https://www. [symbol] |
| | Profiler 96 | | the nitrocellulose membrane. | [symbol] |
| | Microplate-based | | | [symbol] |
| | Antibody Arrays | | | [symbol] |
| Sigma Life sciences | Panorama ® | Single spot | The arrays consist of polyclonal and | https://www. [symbol] |
| | Antibody Array | | monoclonal antibodies are spotted | [symbol] |
| | | | on nitrocellulose-coated glass slides. | [symbol] |
| | | | | [symbol] |
| | | | | [symbol] |
| | | | | [symbol] |

Antigen microarray

| | | | | |
|---|---|---|---|---|
| GeneCopoeia | GeneCopoeia's | Single spot | Antigens are spotted onto | https://www. [symbol] |
| | OmicsArray ™ | | nitrocellulose filters, which are | [symbol] |
| | | | adhered to glass slides. | [symbol] |
| Development of an | Microgrid II | Single spot | Biotinylated proteins are spotted at | https://www. [symbol] |
| antigen microarray | arrayer | | the base of streptavidin-coated 96- | [symbol] |
| for high throughput | (BioRobotics) | | well microtiter plates. | [symbol] |
| monoclonal | | | | |
| antibody selection | | | | |
| An antigens | Microgrid | Single spot | Antigens are printed on an | Nat. Protoc. 5, 1932- |
| microarray | Compact | | aldehyde-coated slides. | 1944 (2010). |
| immunoassay for | (Biorobotics) | | | https:// [symbol] |
| multiplex screening | | | | [symbol] |
| of mouse | | | | |
| monoclonal | | | | |
| antibodies | | | | |
| Generation of | V [symbol] Array | Single spot | Antigens including proteins, | PLOS ONE 11(3): |
| Antigen | Chipwriter Pro | | peptides, and lysates are spotted | [symbol] |
| Microarrays to | microarrayer | | onto two-pad FAST nitrocellulose | [symbol] |
| Screen for | (Virtek, Canada) | | coated slides (Maine | [symbol] |
| Autoantibodies in | | | Manufacturing, Sanford, ME). | |
| Heart Failure and | | | Solid Pins (Arrayit, Sunnyvale, | |
| Heart | | | CA) are used to generate features | |
| Transplantation | | | of approximately 500 microns in | |
| | | | diameter. | |

Antibody and Antigen microarray

| | | | | |
|---|---|---|---|---|
| Combinatory | MicroGrid II | Spot-on-spot | Capture antibody and peptides are | PLoS One. 2016 Dec. |
| Antibody± Antigen | arrayer | [symbol] | printed on a microarray slide onto | [symbol] |
| Microarray Assay | (BioRobotics) | | N-hydroxysuccinimide (NHS)- | [symbol] |
| for High-Content | | | activated glass array slides | [symbol] |
| Screening of | | | (SCHOTT NEXTERION ®Slide H) | [symbol] |
| Single-Chain | | | before adding scFv in a spot-on- | |
| Fragment Variable | | | spot print. | |
| Clones from | | | | |
| Recombinant | | | | |
| Libraries | | | | |

DNA/Oligonucleotide Microarray

| | | | | |
|---|---|---|---|---|
| Immobilization | | Self- | Thiol-modified DNA probes are | Sensors 2014, 14, |
| Techniques for | | assembly | immobilized on gold electrodes | 22208-22229: |
| Microarray: | | | through a self-assembly process. | doi:10.3390/s141222208 |
| Challenges | | | | |
| and Applications | | | | |
| Identifying | MicroGrid II | Single spot | PCR products are diluted in print | Antimicrobial Agents |
| Antimicrobial | arrayer | | buffer heat, denatured with a | and Chemotherapy, |
| Resistance Genes | (BioRobotics) | | thermal cycler, and allowed to cool | October 2003, p. 3290- |
| with DNA | | | to room temperature. Each probe | 3295. |
| Microarrays | | | was then deposited onto epoxy- | |
| | | | silane-derivatized, Teflon-masked | |
| | | | slides by using a robotic spotter. | |
| Preparation of | Spotting robot | Single spot | Oligonucleotide or PCR products | Nucleic Acids Res. |
| DNA and protein | (GeneMachines ™) | | are spotted onto activated agarose | 2000: 28(12): E66. |
| micro arrays on | | | film using a pin-tool based spotting | doi:10.1093/ [symbol] /28.12.e66 |
| glass slides coated | | | robot. | |
| with an agarose | | | | |
| Agilent | In situ- | Base-by- | Uses glass slides and inkjet | Clinical Microbiology |
| Technologies | synthesized | base printing | printing. Noncontact inkjet printing | Revies, 2009, 2009 |
| | oligonucleotide | | technology delivers a small volume | p. 611-633. |
| | microarray via | | (picoliters) of nucleotides to the | |
| | noncontact inkjet | | first layer on the microarray | |
| | printing | | surface. Repeated rounds of base- | |
| | | | by-base printing extend the length | |
| | | | of specific oligonucleotide probes | |
| | | | with final product of a 60-mer in | |
| | | | situ-synthesized probe, | |

Simultaneous antibody, antigen, and oligonucleotide
microarray printing on a single chip

| | | | | |
|---|---|---|---|---|
| Luna Labs | SPRi-Continuous | MultiSpot ® [symbol] | Thiolated protein A is printed onto | The present disclosure |
| | Fluidic | | a gold film surface, added with | |
| | Microspotter | | capture antibody (CAb) via a spot- | |
| | (CFM HORIBA | | on-spot print, and finally printed | |
| | Scientific) | | with antigen on top of CAb with | |
| | | | sinmltaneous spotting with | |
| | | | thiolated capture aptamer on the | |
| | | | metal chip surface. Capture probes | |
| | | | in both [symbol] acid (RNA/DNA) | |
| | | | and protein (antibodies, antigens) | |
| | | | forms are printed simultaneously | |
| | | | on a chip. | |

[symbol]

indicates data missing or illegible when filed

As discussed herein, the present disclosure provides an improved surface functionalization, printing, and blocking system (i.e., SAS) that can construct sensing arrays and prevent non-specific binding of biomacromolecules to allow precise detection of low concentration target biomarkers in human serum samples. The functionalization technique typically involves either (1) the use of a press load to achieve a uniform protein A coating across the chip surface or (2) the use of an automated printer, CFM to spot protein A on the chip surface and stack it up with capture probes (e.g., antibody, and antigen). The blocking system can include reagents with appropriate concentrations listed in Table 4 to allow effective blocking of undesirable, abundant proteins.

TABLE 4
List of reagents and corresponding concentrations
investigated for SAS.

| Reagents | Concentration Range | |
|---|---|---|
| Thiolated polyethylene glycol (MW = 1000 | 1-10 | mM |
| g/mol) | | |
| Thiolated polyethylene glycol (MW = 2000 | 0.1-10 | mM |
| g/mol) | | |
| Human IgG, mixed IgG subtypes (human protein | 10-300 | μg/mL |
| blocker) | | |
| Rabbit IgG, mixed IgG subtypes (rabbit protein | 10-300 | μg/mL |
| blocker) | | |
| Mouse IgG, mixed IgG subtypes (mouse protein | 10-100 | μg/mL |
| blocker) | | |
| Bovine serum albumin (BSA) | 0.1-5% | (w/v) |
| Human Fc fragment, mixed IgG subtypes | 0.1-10 | μg/mL |
| Phosphate buffered saline (PBS) | 1-10× | |
| Decafluoro-1,1,2,2,-tetrahydrooctyl | 0.5-1% | |
| trichlorosilane (FOTS) | | |
| Lipid bilayer, egg phosphatidylcholine (EPC) | 0-100% | |
| Lipid bilayer, palmitoylo-leoylphosphatidylcho- | 50-100% | |
| line (POPC) | | |
| Mercaptopropanol (MPO) | 0.5-5 | mM |

| | |
|---|---|
| Triton X | 0.5-5% |
| Tween 20 in PBS buffer | 0.05-1% |
| Tween 20 in PBS buffer | 0.05-1% |

The novel blocking system herein preferably consists of about 4 mg/mL thiolated polyethylene glycol (PEG-SH), about 10 μg/mL IgG mix (consists of rabbit IgG, human IgG, and/or mouse IgG), about 1% (w/v) bovine serum albumin (BSA), and about 0.1-0.5 μg/mL Fc fragment in about 1-2× phosphate-buffered saline (PBS) solution. Hypothetically, these blocking agents work synergistically in a way that the terminal thiol groups of PEG will tether onto the remaining gold layer that was not covered by thiolated protein A; while the IgG, BSA and Fc fragment together will block the exposed regions of protein A, capture antibody, antigen, and control antibody. In this manner, only capture probe spots specific for target biomarker or indicator will be allowed to interact. Target indicators will then be detected and amplified. Since the analysis involves human serum, human biomarker, and capture and detection antibodies (rabbit monoclonal, or recombinant), the IgG blocking mix consisting of rabbit, human, and/or mouse IgG was employed to further optimize blocking. These strategies employed on biosensor surface allow biomarker analyses in serum with low sample volume requirement, dye-free, purification-free, high sensitivity (pg/mL limit of detection) and real-time multiplex monitoring using SPRi-based detection platform.

SAS is Designed for Use as a Universal Surface Activation System (SAS) Compatible with any Type of Surfaces Such as Metals, Glass, Ceramic, and Polymers.

Luna Labs has demonstrated the use of SAS for multiplex detection (See Examples section) of a number of biomarkers (e.g., proteins, engineered antibodies, nucleic acids and extracellular vesicles) and pathogen indicators (e.g., Zika, Dengue, or Chikungunya) in different complex sample matrices such as clinical serum samples, animal serum samples and supernatants. SAS can be extended for the analyses of other matrices such as blood, urine, saliva, semen, and many others. For example, the SAS described herein can be used to treat sensor chip surfaces for the analysis of traumatic brain injury biomarkers in cerebrospinal fluid and methylated genes associated with post-traumatic stress disorder (PTSD) in genomic DNA extracted from whole blood samples.

The SAS protocol can be implemented across multiple other relevant surfaces where functionalization and blocking are required. The thiol moiety of functionalization agent (protein A) can be replaced with other functionalities (amine, carboxyl or hydroxyl group) to be able to perform other types of reactions (such as carbodiimide-based modification, siloxane network formation, etc.) on the surface-active site. The blocking mixture of SAS can potentially be useful for protein A coated plates and beads implemented in ELISA. SAS could also be used for impedance-based biosensors that use gold surfaces and be adapted for blocking alternative solid supports for assays such as electrodes, fiber optics, microplates, and magnetic/polymeric particles.

The array printing herein can be used to simultaneously immobilize different proteins (e.g., antibodies, antigens) as well as nucleic acids on a sensing chip surface. These proteins can be spotted on top of the functionalization agent such as protein A, protein G. protein L, or streptavidin.

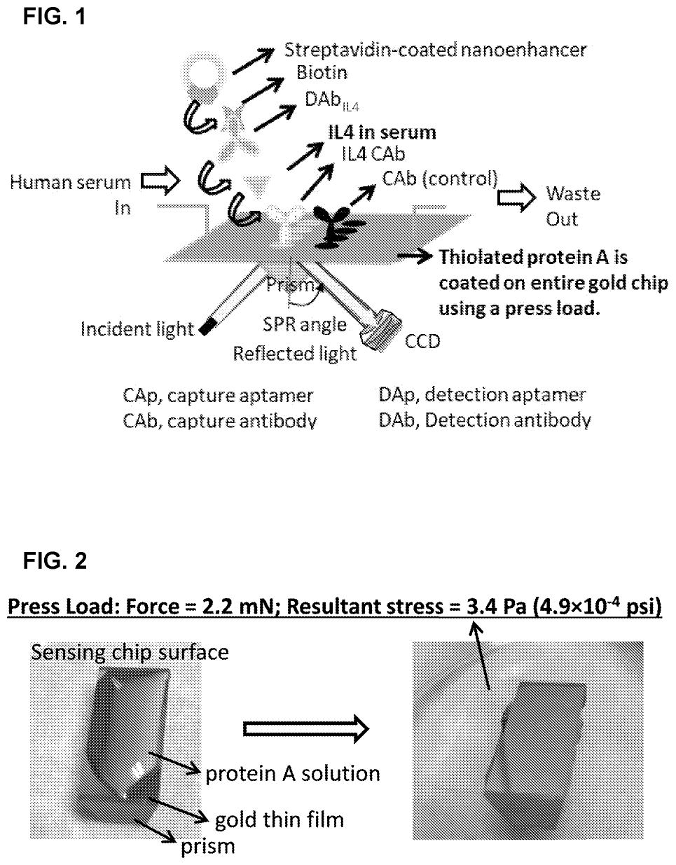

Surface Plasmon Resonance imaging (SPRi) is a phenomenon that occurs when polarized light hits a metal film at the interface of media with different refractive indices (FIG. 1). SPRi techniques excite and detect collective oscillations of free electrons (known as surface plasmons) via the Kretschmann configuration, in which light is focused onto a metal film through a glass prism and the subsequent reflection is detected. The resonance angle can be obtained by observing a dip in SPRi reflection intensity. A shift in the reflectivity curve is characteristic of a specific molecular binding event taking place on or near the metal film. By monitoring this shift vs. time, researchers can study molecular binding events and binding kinetics without the inconvenience of labels. Since the plasmon wave is on the boundary of the conductor and the external medium (e.g., air, water, or vacuum), these oscillations are very sensitive to any change of this boundary, such as the adsorption of proteins to the conducting surface. As the biomolecules bind to the sensor surface, the reflectivity close to the surface changes. The change in SPRi angle is proportional to the mass of material bound.

Like ELISA, the SPRi-based assay involves a sandwich assembly of receptor/ligand matched pair. The general scheme for detection of a biomarker (e.g., cytokine IL4) is presented in FIG. 1. The assays can accommodate up to 400 sample spots (such as proteins, nucleic acids and exosomes) to enable high-throughput biomarker detection. In this type of assay, biotinylated detection antibody (biorecognition probe) is used as the detection system while streptavidin-coated nanoenhancer” quantum dot (QD) is utilized as the signal amplification technique. When QD is covalently bound to the detection antibody via biotin-streptavidin interaction, QD adds mass to the sandwich construct resulting in improved SPRi signal detection. A portable SPRi spectrometer with a highly sensitive CCD camera is used to capture the reflectivity images of each spot of the array. The sensor surface, the microfluidic system, and the SPR detection/imaging unit work together to measure biomolecular interactions. The results from the detection of changes in reflectivity of p-polarized light at a fixed angle is displayed as a “sensorgram” with the change in reflectivity on the y-axis is plotted against time on the x-axis. Binding, specificity, affinity, kinetics and active binding concentration can be determined from the shape of the produced sensorgram.

The overall performance of SPRi-based assay is highly dependent on three steps (Table 5): (1) the surface functionalization, and (2) surface printing to achieve the desired anchoring of the sandwich construct for biomarker or indicator detection and (3) the surface blocking to prevent non-specific binding of proteins and achieve high S/N ratio.

Two different techniques of functionalization are shown herein to illustrate some aspects of the present disclosure. First technique is the use of a press load to achieve a uniform thiolated protein A coating across the gold chip surface. Second technique is the utilization of an automated printer CFM to spot thiolated protein A on specific area of the gold surface which in turn leads to a unique approach of simultaneous antigen and DNA array printing, a MS (multiple spotting) technology.

Table 5 summarizes the coverage of SAS for the single detection of a biomarker cytokine and multiple detection of pathogen indicators of Zika virus (ZIKV). Both functionalization techniques involve the blocking treatment on the chip surface. Printed microarrays are blocked in a blocking buffer to minimize non-specific binding.

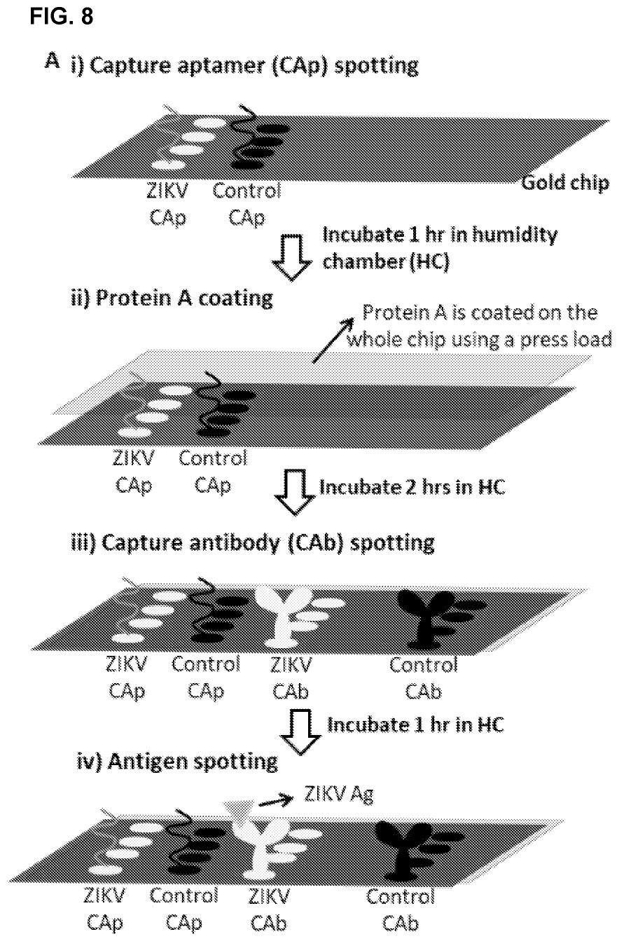

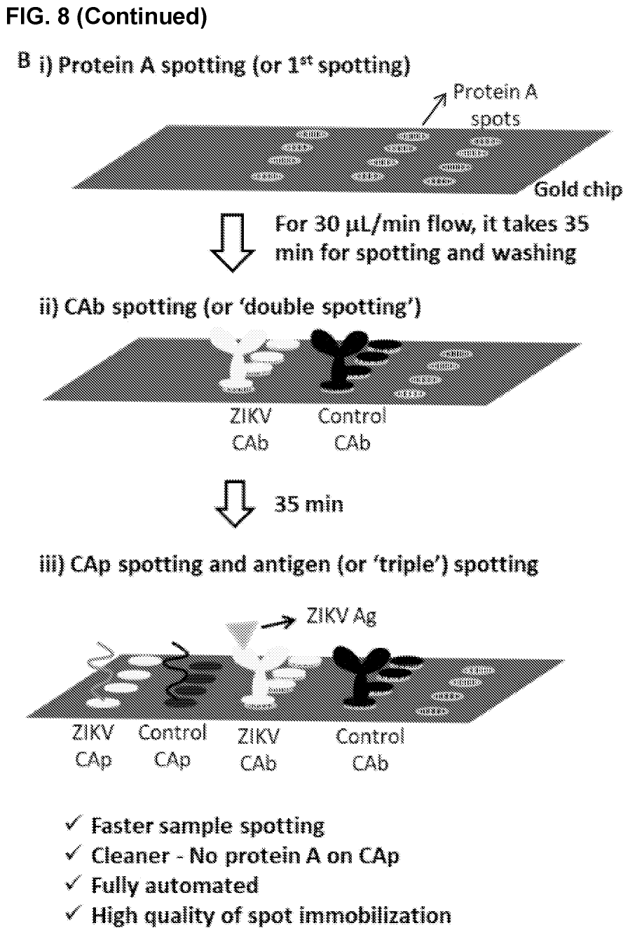

TABLE 5
Established SAS protocols for the detection of 2 different types of target markers in human serum sample.

| Marker | Coverage of Surface Activation System of the Present Disclosure | | |
|---|---|---|---|
| in serum | (1) Functionalization | (2) Array Printing | (3) Blocking |
| I. Biomarker | Thiolated protein A is | Antibody microarray: | (i) 4 mg/mL thiolated methoxy |
| cytokine | coated across the gold | Recombinant IL4 antibody is | polyethylene glycol (mPEG, |
| interleukin 4 | chip with the aid of a | manually printed on the surface | MW of 2,000 g/mol), (ii) 10 μg/mL |
| (IL4) | press load (see FIGS. | of immobilized protein A. | IgG mix (1:1 molar ratio |
| | 2 and 8A). | | of rabbit IgG and human IgG), |
| | | | (iii) 1% (w/v) BSA, and (iv) 0.5 μg/mL |
| | | | Fc fragment in 1 × PBS. |
| II. Pathogen | By using an | Simultaneous printing of | (i) 4 mg/mL thiolated mPEG |
| indicators of | automated | antigen and DNA microarrays | with MW of 2,000 g/mol, (ii) 10 μg/mL |
| ZIKV: RNA, | microspotter (CFM), | (using a MS (multiple spotting) | mouse IgG, (iii) 1% |
| IgM, and IgG | thiolated protein A is | technology)): Recombinant | (w/v) BSA, and (iv) 0.1 μg/mL |
| | printed on a specific | antibody is automatically | Fc fragment in 2 × PBS. |
| | region of the chip | printed on the surface of the | |
| | (see FIG. 8B). | spotted protein A followed by | |
| | | simultaneous spot-on-spot | |
| | | antigen printing on CAb and | |
| | | capture aptamer spotting on | |
| | | gold surface. | |

### Definitions

As used herein, the singular form “a”, “an”, and “the”, includes plural references unless it is expressly stated or is unambiguously clear from the context that such is not intended.

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. As used herein, when “about” is used to modify a range, both the lower limit and higher limit should be understood as preceding with the term “about”, and the lower limit and higher limit should have the same unit unless otherwise specified. For example, about 1-5 mM should be understood as about 1 mM to about 5 mM. Whether or not modified by the term “about”, the claims include equivalents of the recited quantities. In one embodiment, the term “about” means within 20% of the reported numerical value. The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A. B, or C; A or C; A or B; B or C; A and C; A and B; B and C; A (alone); B (alone); and C (alone).

As used herein, “% sequence identity” or “percent identity” refers to protein sequence identity. Percent identity may be determined using standard techniques known in the art. Useful algorithms include the BLAST algorithms (See, Altschul et al., J Mol Biol. 215:403-410, 1990; and Karlin and Altschul, Proc Natl Acad Sci USA, 90:5873-5787, 1993). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402 (1997); and Schaffer et al., Nucleic Acids Res, 29:2994-3005 (2001). Exemplary default BLAST parameters for a nucleic acid sequence searches include: Neighboring words threshold=11: E-value cutoff=10: Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. A percent (%) amino acid sequence identity value is determined by the number of matching identical residues divided by the total number of residues of the “reference” sequence including any gaps created by the program for optimal/maximum alignment. BLAST algorithms refer to the “reference” sequence as the “query” sequence.

The term “antibody” is used herein in the broadest sense and encompasses monoclonal antibodies (including full length monoclonal antibodies), polyclonal antibodies, multi-specific antibodies (e.g., bispecific antibodies), and antibody fragments so long as they exhibit the desired biological activity. “Antibodies” (Abs) and “immunoglobulins” (Igs) are glycoproteins having the same structural characteristics. Antibodies include immunoglobulin molecules and immunologically active fragments of immunoglobulin molecules, namely, molecules that contain an antigen binding site (e.g., a Fv, a Fab, a Fab′, a F(ab′)₂, dsFv, Fd, scFv, and diabodies), or a single-domain antibody. Immunoglobulin molecules can be of any type (for example, IgG, IgE, IgM, IgD, IgA and IgY), class (for example, IgG₁, IgG₂, IgG₃, IgG₄, IgA₁ and IgA₂) or subclass. Suitable antibodies can be generated by any animal (e.g., a bird (e.g., duck, chicken, goose, etc.); a shark; a fish (e.g., zebrafish); a mammal (e.g., a non-primate, e.g., cow, pig, camel, llama, horse, goat, rabbit, sheep, hamster, guinea pig, pig, cat, dog, rat, mouse, etc.; a non-human primate, e.g., monkey, cynomolgus monkey, chimpanzee, etc; a human; etc.), and the like.

The term “alternative binding scaffold” is used herein in the broadest sense and encompasses polypeptides containing alternative binding scaffolds that are able to specifically bind a target antigen of interest with high affinity. An alternative binding scaffold may include, but is not limited to, an affibody, nanobody, anticalin, fynomer, DARPin, Tetranectin, Transbody, AdNectin, Affilin, Microbody, peptide aptamer, alterase, plastic antibody, phylomer, stradobody, maxibody, evibody, Z domain, D domain, armadillo repeat protein, Kunitz domain, avimer, atrimer, probody, immunobody, triomab, troybody, pepbody, vaccibody, UniBody, Affimer, or a DuoBody. Alternative binding scaffolds have uses that include but are not limited to capture molecules and detection molecules as provided herein.

The term “aptamer” is used herein to refer to oligonucleotides (e.g., short oligonucleotides, deoxyribonucleotides, or ribonucleotides) or peptides, that bind (e.g. with high affinity and specificity) to proteins, peptides, and small molecules. Oligonucloetide aptamers may be single stranded or double stranded. The aptamers provided herein are generally fewer than 100 nucleotides, fewer than 75 nucleotides, or fewer than 50 nucleotides in length. The provide aptamers often have Kd's in the nM or pM range, e.g. less than one of 500 nM, 100 nM, 50 nM, 10 nM, 1 nM, 500 pM, 100 pM. Aptamers have uses that include but are not limited to capture molecules and detection molecules (“capture aptamer(s)” and “detection aptamer(s),” respectively).

In the present specification, the nucleic acid constituting the oligonucleotide aptamer is not particularly limited. The nucleotides are preferably selected from AMP, TMP, GMP, UMP, dAMP, dTMP, dGMP or dCMP. The nucleotides may be abasic (i.e. lack a nucleobase).

The aptamer may comprise chemically modified nucleotides or nucleosides, for example one or more chemical substitution at a sugar position, a phosphate position, and/or a base position of the nucleic acid including, for example, incorporation of a modified nucleotide, peptide nucleic acid (PNA), a peptide nucleic acid having a phosphate group (PHONA), a bridged nucleic acid or locked nucleic acid (BNA or LNA), and a morpholino nucleic acid, incorporation of a capping moiety (e.g., 3′ capping), conjugation to a high molecular weight, non-immunogenic compound (e.g. polyethylene glycol (PEG)), conjugation to a lipophilic compound, substitutions in the phosphate backbone. In particular, suitable modified nucleotides include, but are not limited to. 4′-thio pyrimidines (such as 4′-thio uridine and 4′-thio cytidine) and nucleotides having modifications of the nucleobase (such as 5-pentynyl-2′-deoxy uridine, 5-(3-aminopropyl)-uridine and 1.6diaminohexyl-N-5-carbamoylmethyl uridine). Base modifications may include 5-position pyrimidine modifications, modifications at exocyclic amines, substitution of 4-thiouridine, substitution of 5-bromo-or 5-iodo-uracil, backbone modifications. Sugar modifications may include 2′-amine nucleotides (2′—NH₂; e.g., 2′amino pyrimidines (such as 2′-amino cytidine and 2′-amino uridine), 2′-fluoro nucleotides (2′-F; e.g., 2″-fluoro pyrimidines (such as 2′-fluorocytidine and 2′-fluoro uridine), hydroxyl nucleotides (such as 2′-hydroxyl purines) and 2′-O-methyl (2′-OMe) nucleotides (such as, 2′-O-methyl adenosine, 2′-O-methyl guanosine, 2′-O-methyl cytidine and 2′-O-methyl uridine), hydroxyl pyrimidines (such as 5′-a-P-borano uridine),

The nucleotides or nucleosides making up the aptamer nucleic acids may include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate. 5-methylcytidine triphosphate, 5-hydroxymethylcytidine monophosphate, S-hydroxymethylcytidine diphosphate, 5-hydroxymethyl-cytidine triphosphate, cyclic adenosine monophosphate (CAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxygeanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, S-methyl-2′-deoxycytidine triphosphate, 5-hydroxymethyl-2′-deoxycytidine monophosphate, 5-hydroxymethyl-2′, deoxycytidine diphosphate and 5-hydroxymethyl-2′-deoxycytidine triphosphate. The nucleotides may contain additional modifications.

A wide range of nucleotide, nucleoside, base and phosphate modifications are known to those or ordinary skill in the art, e.g. as described in Eaton et al., Bioorganic & Medicinal Chemistry, 5 (6): 1087-1096 (1997). In some embodiments, the “aptamer” is a mirror-image aptamer(s) (e.g., containing high-affinity L-enantiomeric nucleic acids such as, L-ribose or L-2′-deoxyribose units) that confer resistance to enzymatic degradation compared to D-oligonucleotides.

As used herein, a “capture molecule” or “capture probe” is any molecule that is capable of binding to an analyte (i.e. capturing it). Suitable capture molecules include, without limitation, a protein or polypeptide, a nucleic acid molecule, or an organic small molecule probe. In particular embodiments, the capture molecule is an antibody, antigen binding fragment of an antibody, an aptamer, or an alternative binding scaffold. It is desirable that the capture molecule binds specifically to the analyte of interest.

As used herein, an “immobilized” reagent, refers to the reagent that will normally remain on a surface after addition of a sample during the conduct of an assay, although there may be specific conditions that can be used to actively dissociate it from the surface.

As used herein, the term “blocking”, “to block” (e.g., as in the phrase “blocking agent”) refers to preventing non-specific binding from occurring, to a target other than the intended target, when using a one or more (e.g., a cocktail) of antibodies and allowing the specific binding to occur. For example, many antibodies bind to non-target antigens (secondary antigens) (e.g., with lower affinity) in addition to the target antigen (the primary antigen) to which they specifically bind with high affinity. Some species of antibody and individual antibodies can have sticky binding characteristics and thus increase background signal.

The terms “specific binding,” “specifically binds,” and the like, refer to the preferential binding of a molecule (e.g., one binding pair member to the other binding pair member of the same binding pair) relative to other molecules or moieties in a solution or reaction mixture. The term “specific binding member” refers to a member of a specific binding pair. Exemplary specific binding members include, but are not limited to ligand/receptor; antibody/antigen; nucleic acid sequence/complementary strand, and the like. Specific binding members can be proteins (e.g., peptides, polypeptides, etc.), fusion proteins, antibodies, etc. In some embodiments, the affinity between a pair of specific binding members when they are specifically bound to each other in a binding complex is characterized by a K_{D} (dissociation constant) of 10⁻⁵ M or less, 10⁻⁶ M or less, such as 10⁻⁷ M or less, including 10⁻⁸ M or less. e.g., 10⁻⁹ M or less, 10⁻¹⁰ M or less, 10⁻¹¹ M or less, 10⁻¹² M or less, 10⁻¹³ M or less, 10⁻¹⁴ M or less. 10⁻¹⁵ M or less, including 10⁻¹⁶ M or less. “Affinity” refers to the strength of binding, increased binding affinity being correlated with a lower K_{D}.

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### Examples

### Example 1. Surface Functionalization with Protein A

This example shows typical procedures for surface functionalization of a gold surface with protein A. As discussed in FIG. 1, the metal surface is preferably functionalized with an immobilization matrix, which in this Example is the protein A, to achieve sandwich assembly of antibody/antigen matched pair on the sensing chip. The terminal moiety of protein A is modified with a thiol group to allow binding with the gold thin film (50 nm-100 nm thick) of the biosensor chip. The binding pocket of protein A is the foundation of the sandwich construct, which can immobilize the capture antibody (CAb) at a proper orientation (i.e., through antibody's Fc region).

Prior to functionalization, the gold thin film surface was cleaned with a strong oxidizer (piranha solution) to turn gold surface into gold oxide. Thiols in protein A were allowed to react directly with this oxidative gold surface to form Au—S bonds via reduction of the gold oxide and the direct adsorption of thiols onto the reduced gold surface.

For efficient functionalization on the surface, a 10 μg/mL thiolated protein A solution in PBS buffer, pH 7.4 is preferred.

The surface functionalization may be performed with or without a press load. However, it was found that the use of a press load can lead to more uniform treatment across the chip surface.

Functionalization with a press load: A press load was placed on top of the treatment solution (a 10 μg/mL thiolated protein A solution in PBS buffer, pH 7.4) to press protein A on the gold surface of the chip with the application of appropriate force (2 mN) and resultant stress (3 Pa or 5×10⁴ psi). The press load is a 1″×1″ (25 mm×25 mm) glass cover slip of 0.5 mm in thickness and possesses the suitable force and stress needed for functionalization. See also FIG. 2.

Before use, the press load is carefully washed twice with ethanol and then with de-ionized water and is dried using stream of N₂. Once the solution is pipetted onto the gold layer, it is compressed with the cleaned press load to ensure complete and uniform contact with the metal surface. Excess solution escapes from under the press load, leaving uniform protein A treatment across the surface.

Functionalization without a press load: the same process is followed as above except that the press load is not used during functionalization of the surface. See also FIG. 2.

### Example 2. Surface Blocking

This example shows exemplary blocking system to eliminate non-specific binding of proteins on the sensing surface. Subsequent to functionalization, the capture antibody is spotted on the chip and allowed to dry under a humidity chamber (see detailed protocol in Example 3 below). The chip is then mounted onto the SPRi instrument and successive injections of blocking agents are carried out.

The novel blocking system preferably consists of 4 mg/mL thiolated methoxy polyethylene glycol (mPEG-SH, MW of 2k), IgG mix (1:1 molar ratio rabbit IgG and human IgG, 10 μg/mL each), 1% (w/v) bovine serum albumin (BSA) and 0.5 μg/mL Fc fragment in 1× phosphate-buffered saline (PBS) solution. These blocking agents work synergistically in a way that the terminal thiol groups of PEG will tether onto the remaining gold layer that was not covered by thiolated protein A; while the IgG, BSA and Fc fragment together will block the exposed regions of protein A, capture antibody and control capture antibody. In this manner, only capture antibody spots specific for target biomarker will be allowed to interact, be detected and amplified. Since the analysis involves human serum, human biomarker and rabbit monoclonal antibodies for capture antibody and detection antibody, an equimolar ratio of human IgG and rabbit IgG for the IgG mix was employed to further optimize the blocking condition.

All blocking steps are done in-line (i.e., injection of reagents in the flow cell) at a flow rate of 20 μL/min for ˜30 min using 1×PBS running buffer.

For partial blocking, the IgG mix (human IgG and rabbit IgG) is not used.

### Example 3. Preparing and Blocking SPRi Chips for Biomarker Detection

The following shows a general procedure for the detection of biomarker (e.g., IL4) in human serum using SPRi chips. It is expected and assumed that an identical or similar protocol can be implemented across multiple other relevant surfaces where functionalization and blocking are required.
1. Proper functionalization requires a clean surface. Wash re-usable, bare, untreated gold chips with ethanol and de-ionized (DI) water and dry with a gentle stream of N₂ gas. Immerse chips in piranha solution with 3:1 (v/v) H₂SO₄: H₂O₂ for 50 min with gentle sonication. Take chips out of the solution, rinse with running DI water for 5 min and ethanol and dry with N₂ stream. Expose chips to UV-Ozone (Jelight Company, Inc.) twice to afford formation of hydrophilic surface.
2. Carefully wash press load with ethanol twice and then with DI water. Dry it using a stream of N₂. Apply 64 μL 10 μg/mL of thiolated protein A solution per cm² of the gold layer of the chip. Place the cleaned press load onto the solution and avoid any bubble formation between interfaces. The force (2 mN) and the resultant stress (3 Pa or 5×10⁻⁴ psi) of the load allow compression of the solution to make efficient contact with the gold thin film. Excess solution escapes from under the press load cover slide, leaving only a certain amount to ensure uniform formation of the protein A layer on the sensing surface. Incubate for 2 hrs in a humidity chamber (65-75% relative humidity) to allow the binding of thiol molecules on the metal surface. Wash the chip with DI water and dry the chip with N₂ stream. Allow chip to form self-assembly of protein A layer for at least 3 hrs prior to use.
3. Prepare 10 μL of 300 to 1000 μg/mL capture antibody of IL4. Using a pin with 1 mm tip, dip the pin in the capture antibody solution and spot this solution on top of the previously functionalized protein A layer in 5 replicates. Do the same for the negative CAb control (e.g., 300 to 1000 μg/mL rabbit IgG) and place the chip in a humidity chamber (65-75% relative humidity) for 2 hrs to keep capture antibody spots from drying.
4. Mount the chip onto the SPRi instrument. Throughout the analysis, use 1×PBS as the running buffer and the solvent for each blocking agent preparation. Prepare 4 mg/mL of thiolated PEG and inject this solution into the SPRi flow cell. Prepare a mixture of rabbit IgG and human IgG (10 μg/mL each) and inject this mix in the flow cell once PEG signal is stable. Prepare 1% BSA (w/v) and inject this solution in the flow cell once rabbit/human IgG mix signal is stable.

### Example 4. Effect of Press Load on SPRi Assay

To demonstrate the usefulness of a press load in achieving uniform protein A layer on the metal surface, an experiment was carried out using SPRi-based assay. First, a cleaned SPRi chip was functionalized with protein A solution with a press load on top of it. See Example 1. Subsequent to functionalization, capture antibody was spotted (n=5) on the protein A layer using a manual Arrayer (HORIBA Scientific). This was carried out by using a pin with 0.5 mm tip, dipping the pin in the capture antibody solution and manually spotting this solution on top of the previously functionalized protein A layer. Negative control capture antibody was also spotted (n=5). Negative control lacks binding specificity to the target cytokine and should not result in observable signal.

The spotted chip surface was then mounted on SPRi instrument and treated completely with blocking agents, namely, thiolated PEG, IgG mix and BSA (see Example 2). Signals of background (i.e., no capture antibody), capture antibody (CAb, anti-IL4) and control CAb spots were monitored after each blocking agent injection. Three different chips functionalized using the same protocol are presented to demonstrate chip-to-chip reproducibility of protein A treatment.

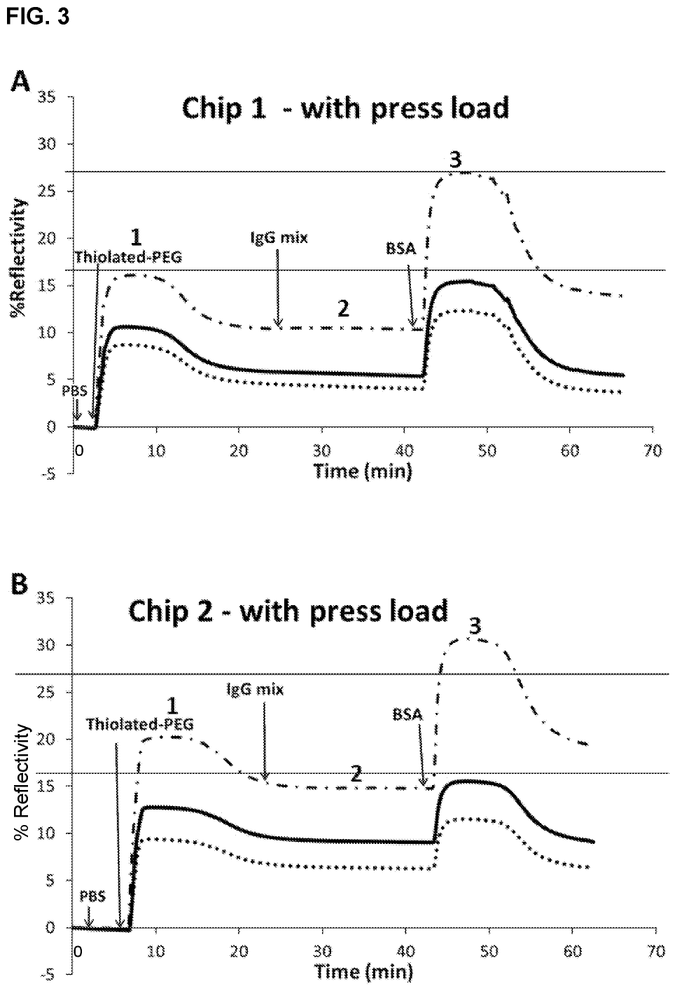

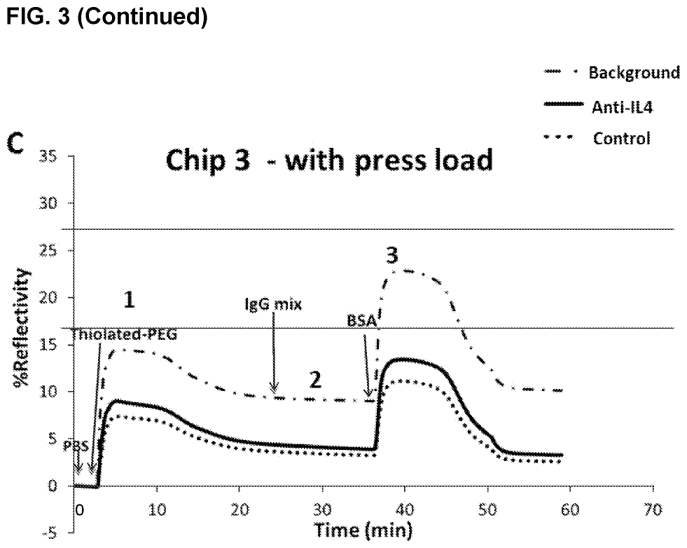

FIGS. 3A-C show the blocking profiles observed on three different chips. For all spots, the consistent signal trend of rise-flat-rise (denoted as 1, 2 and 3, FIGS. 3A-C) was observed after thiolated PEG/IgG mix/BSA injections. The typical increase of signal after thiolated PEG injection indicates the blocking of the surface sites that were not covered with thiolated protein A. Of particular interest is the IgG mix region (point 2) where no signal change is depicted after injection. This suggests minimal adsorption of IgG because of complete surface coverage by functionalized protein A. Highest signal on the background (dash-dotted line) followed by the CAb (solid line) and the control CAb (dotted line) was observed. Highest background signal indicates the highest amount of blocking treatment received since background has the largest unoccupied sites among the 3 spot types. The similarity of signals observed from 3 different chips suggests that the uniformity and density of protein A on the surface are nearly identical after each chip functionalization.

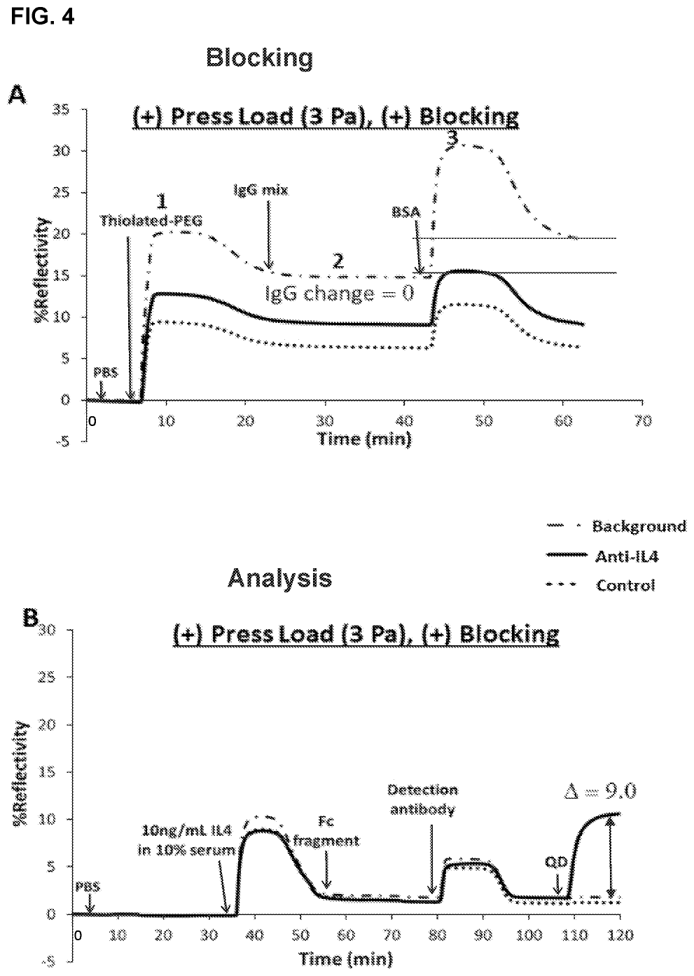

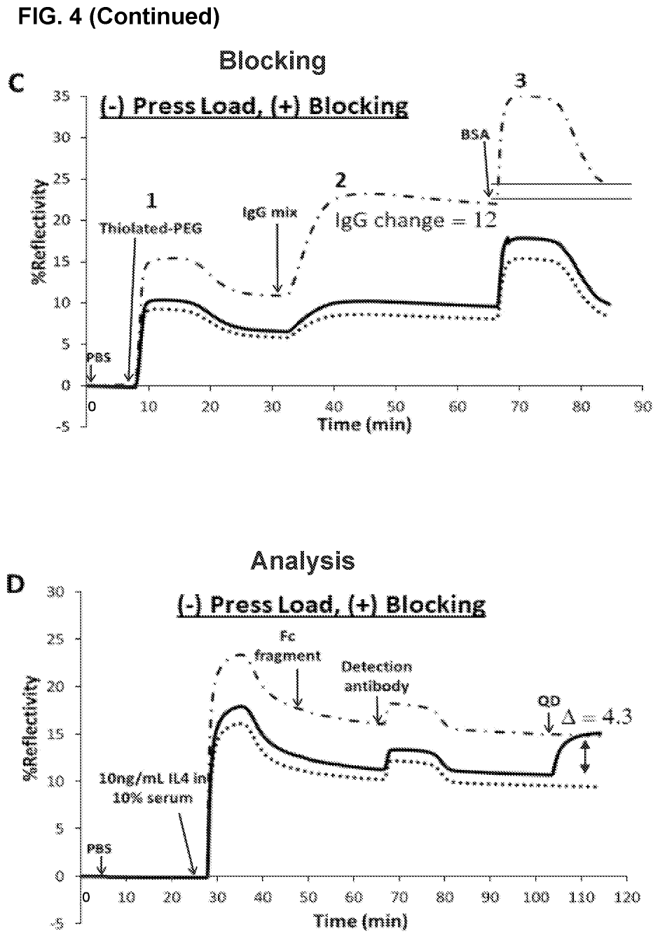

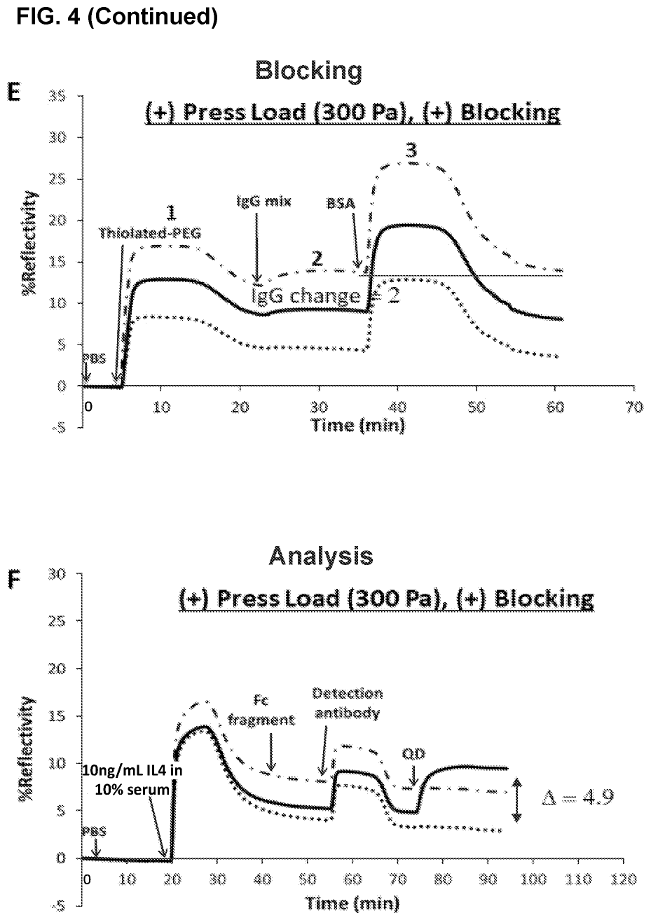

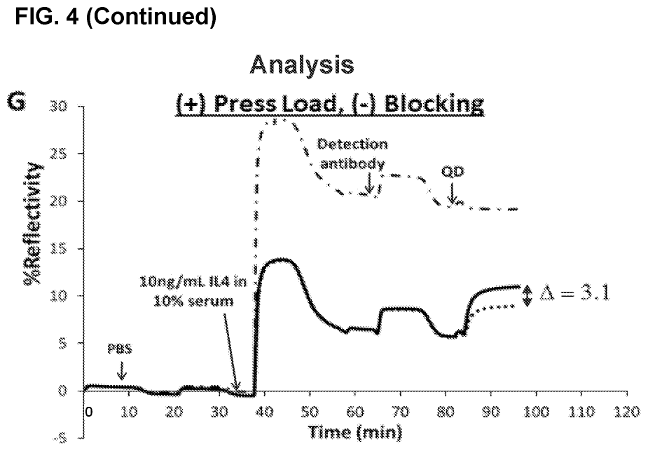

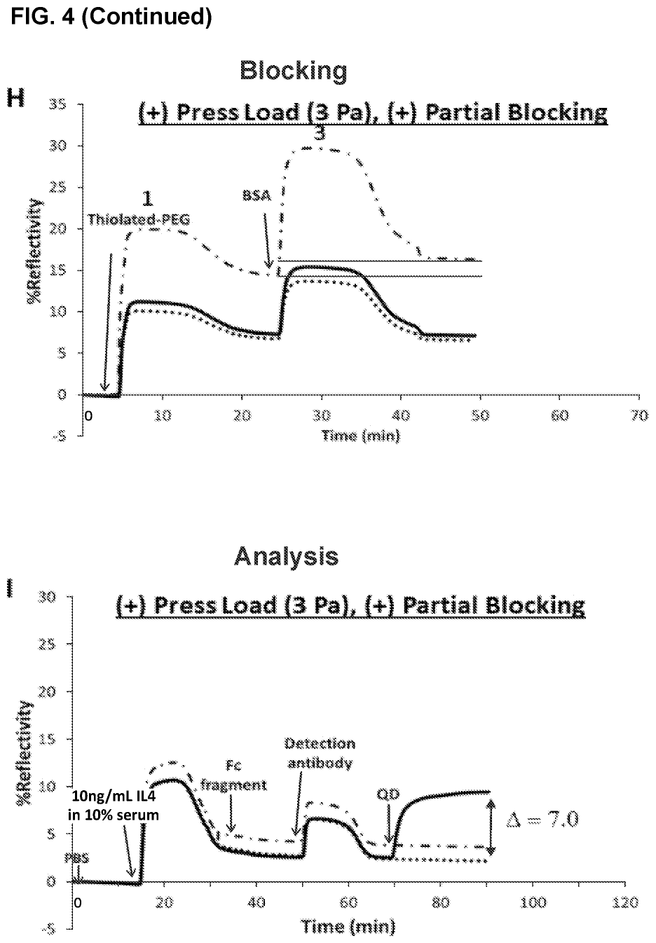

To validate the effectiveness of functionalization, a representative chip with blocking profile presented in FIG. 4A was then used for serum analysis using a validation protocol: 10 ng/ml IL4 spiked in 10% human serum was prepared and injected into the SPRi flow cell. Once signal was stable, final blocking agent, 0.5 μg/mL Fc fragment was injected. Detection of IL4 was carried out by injecting biotinylated anti-IL4 detection antibody followed by 10 nM streptavidin-coated nanoenhancer solution (QD) for signal amplification. The delta value (i.e., control-subtracted reflectivity signal) was then calculated as follows:

Δvalue=[Δ% reflectivity of sample(After QD−Before QD)]−[Δ% reflectivity of control(After QD−Before QD)]

Results show increase of signals (% reflectivity of 10, FIG. 4B) after injection with biomarker (IL4) spiked in 10% human serum. It is normal to observe a signal increase after sample injection because of the non-specific binding of unwanted proteins from the serum constituents. This signal will dramatically decrease if the functionalization and coating on the chip are implemented efficiently.

As shown in FIG. 4B, approximately 20 minutes after injection of the IL4-spike human serum injection, a signal decrease from 10 to 2.5% reflectivity was observed and a baseline similar to that of the control was established. This significant decrease signifies that the abundant non-specific proteins were effectively removed from the surface. The rise of signal after detection antibody injection is normal since the detection antibody is dispersed in a mixture containing 1% BSA, 50% glycerol and 0.05% Tween 20 solution and has a different refractive index than that of the running buffer, PBS. After 10 min, the signal nearly returned to baseline indicating that most of the unwanted proteins/reagents dissociated, leaving only a matched detection antibody to bind with the target IL4. This subtle signal increase from a formed sandwich construct was then amplified by QD.

Interestingly, results in FIG. 4B reveal a significant increase in sample signal (from 2.5 to 11.5% reflectivity) resulting in a very high delta value of 9.0 (with respect to the negative control signal). These results demonstrate that the external force (the press load), assay construction, and blocking agents are essential components to achieve optimal biomarker detection using SPRi biosensor chips.

To demonstrate the effect of detection signal without applying a press load on the surface, the same protein A functionalization protocol was conducted but this time without the use of a press load. Two different chips are presented to demonstrate chip-to-chip reproducibility. Signals of background, CAb and control spots from functionalized chips were also monitored after each blocking injection.

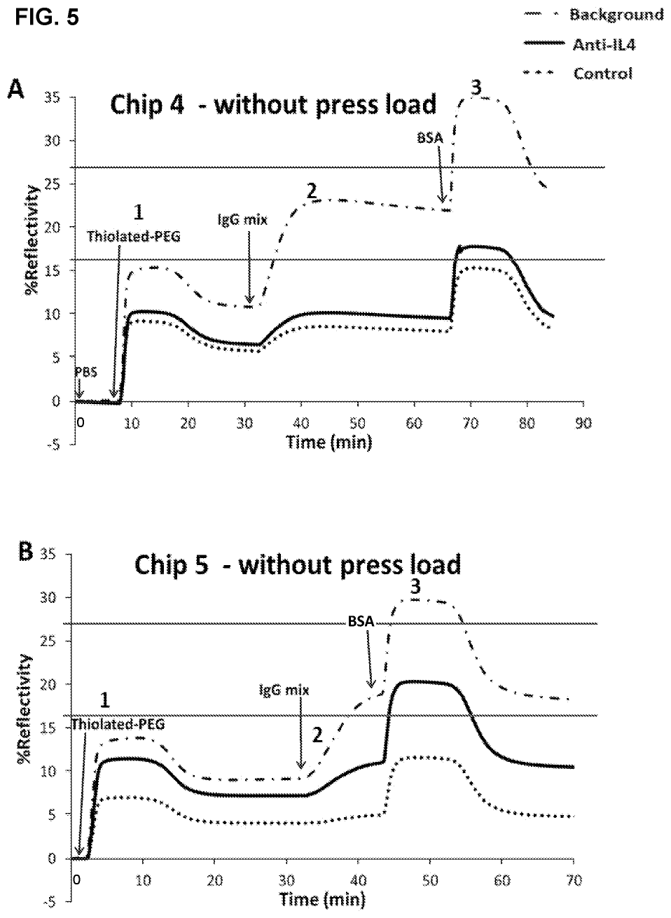

Interestingly, a different blocking profile trend was observed. In particular, the dramatic signal increase after IgG mix injection (point 2, FIG. 5A) was observed. This IgG signal rise may be due to the immobilization of IgG on excess amount of protein A produced from a poor, non-uniform functionalization (i.e., without the use of press load, FIG. 2, left image). This increase was consistently observed on the other chip (FIG. 5B). Moreover, a prominent IgG increase on the capture antibody observed during blocking (point 2, solid line in FIG. 5A) may indicate that the IgG mix blocks the anti-IL4 capture antibody and can result to low target IL4 signal after QD addition.

The chip with the blocking profile presented in FIG. 5A (also presented as FIG. 4C for easy comparison with the corresponding analysis profile) was then analyzed using the validation protocol. Results show a high background signal (% reflectivity of 25, FIG. 4D, dash-dotted line) after injection with IL4 spiked in 10% human serum. This signal stayed high (% reflectivity of 17) throughout the biomarker detection, indicating that the surface did not achieve uniform protein A treatment. Although this chip received a complete blocking treatment, the identified delta value was very low, only 4.3. Without a press load on top of protein A solution, there is no external force that will promote uniform functionalization across the chip surface resulting in poor foundation of the sandwich assembly giving a low biomarker signal. Moreover, the observed high IgG signal increase during blocking (point 2, FIG. 4C) could be used as an indicator for a poor surface functionalization.

Surface Functionalization of the Sensing Chip (with a Heavy Press Load)

To better understand the effect of force and resultant stress on functionalization, a heavy press load exerting 300 Pa resultant stress (2 orders of magnitude higher than the original press load possesses) was utilized. The blocking profile is presented in FIG. 4E while the analysis profile using a validation protocol is shown in FIG. 4F. A noticeable signal increase (A of 2) after IgG mix blocking agent injection was observed. This profile, showing a similar behavior with that observed without the application of press load, indicates poor protein A functionalization. As a result, the A after QD addition is lower (A of 5) than that obtained from the gold standard functionalization protocol (A of 9, FIG. 4B).

These findings suggest that the key to efficient functionalization is to use an appropriate press load force and resultant stress on protein A solution to obtain similar blocking profiles with the ones shown in FIGS. 3A-C and FIG. 4A. These profiles result in increased delta value for the target protein detection (FIG. 4B).

### Example 5. Effect of Blocking on SPRi Assay

To demonstrate the usefulness of the blocking mix in serum analysis, the chip was functionalized with protein A (with the use of a typical press load of 3 Pa) but was not treated with any of the blocking agents. As a result (FIG. 4G), an even higher background signal (% reflectivity of 29.5) was observed throughout the analysis. This condition exhibited a very low S/B signal and a delta value of only 3.1 due to the poor blocking on the sensing surface.

The highest signal observed after sample injection without blocking agent treatment is 30% reflectivity (dash-dotted line, FIG. 4G) while the lowest signal with complete blocking is 2 (dash-dotted line, FIG. 4B). Therefore, the percent removal of non-specific binding proteins using 10% human serum is identified as 93% (>90%), that is, [(30-2)/30*100].

Complete Vs. Partial Blocking

FIG. 4H presents the blocking profile on a surface functionalized with a press load but blocked partially (in this example, all blocking agents applied except for the human IgG and rabbit IgG). The signal after BSA injection is lower than that observed in complete blocking (point 3, FIG. 4A). The obtained delta after QD addition (FIG. 41) shows lower value than that from the golden protocol (FIG. 4B).

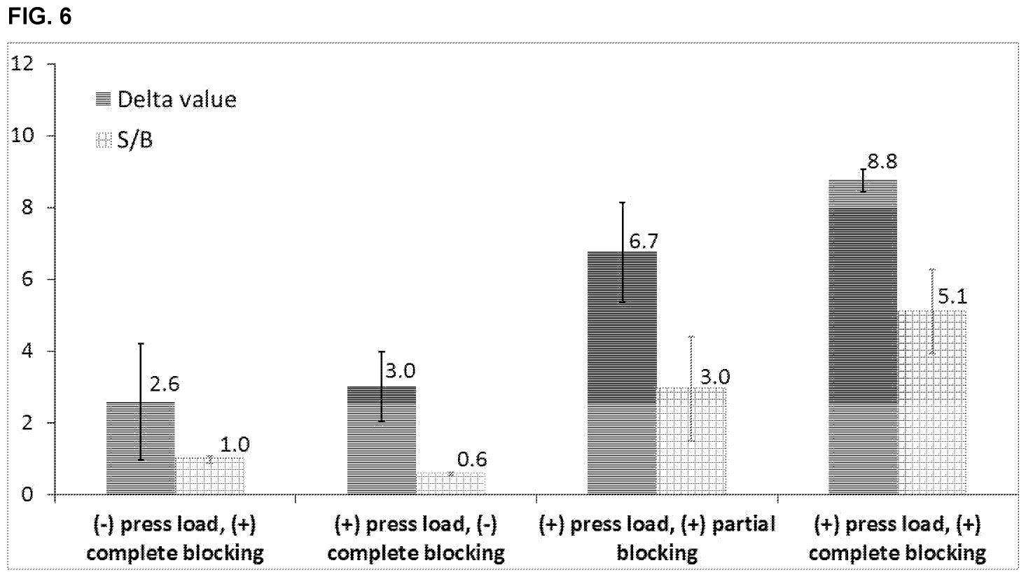

FIG. 6 shows the reproducibility of various conditions employed on the surface of the SPRi-based assay. A total of 12 different chips, ˜60 sample spots and 60 control spots were utilized. The best condition tested (i.e., with press load and complete blocking) reaches a significant average delta value of 8.8. The signal to background ratio (S/B) also exhibits high value of 5 (i.e., 11.5 of IL4 signal÷2.5 of background signal, FIG. 4B). Low background signal is particularly useful for imaging applications.

### Example 6. Continuous Flow Microspotter for Multiplex Detection of Zika Virus

The following section describes the functionalization, array construction, and blocking for the multiplex detection of Zika virus (ZIKV) indicators RNA, IgM, and IgG in human serum sample. This approach uses MS (multiple spotting) technology to functionalize and construct the sensing arrays on the chip surface. Similar blocking approach with the biomarker IL4 presented above was implemented and described below.

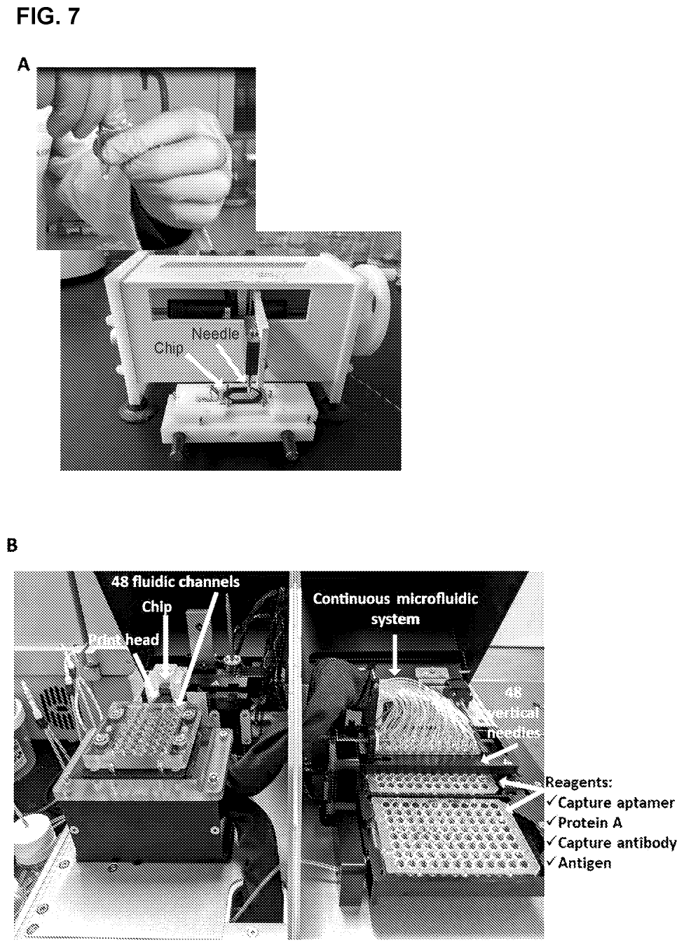

In this example, a robotic printer, the continuous flow microspotter (CFM, HORIBA Scientific) is used for sample spotting. Unlike the typical manual Arrayer that uses a needle to spot sample solution one spot at a time (FIG. 7A), the HORIBA CFM is a fully automated instrument for micro-arraying proteins in a high throughput using continuous sample flow over the spot (FIG. 7B).

### Description of CFM

The CFM has a print head that consists of 48 fluidic channels. Each of these channels creates discrete fluidic path that completely isolates the sample from plate to spot. This enables each channel to be filled with unique sample or replicate. The sample is cycled back and forth through the fluidic channels (100 μm in diameter) at varying spotting periods to efficiently immobilize the protein from the solution. See also “Continuous Flow Microspotter, User Manual, SPRI-CFM 2.5”, HORIBA Scientific, Part Number: 1300042189, 2018, pages 5 and 6, the content of which is herein incorporated by reference in its entirety.

CFM is proven to be more effective (here, 10×better) than the manual Arrayer in immobilizing the capture probes. In the CFM printing technique, the material (e.g., antigen) is cycled back and forth on the surface of the previously immobilized capture probe (e.g. antibody). As this is done continuously during the printing, the frequency of exposing antigen binding sites to the antibody for interaction is higher than the one observed in the Arrayer. Unlike CFM, the Arrayer is governed by static or contact printing. The needle that contains the antigen is dipped onto the antibody surface only once. For this, it is speculated that there is a higher concentration of material that can reach the surface than what is achieved in an Arrayer. Moreover, the immobilization is uniform and clean because the flow washes the surface during immobilization. With CFM, 48 replicates or 48 unique solutions can be spotted to the surface simultaneously.

### A. Surface Functionalization and Construction of Sensing Arrays Using a CFM

The typical steps, using an Arrayer, of functionalizing the chip surface and constructing the sensing arrays for both nucleic acid and protein (e.g., IgG and IgM) detections are presented in FIG. 8A (i-iv). These steps include the spotting of the thiolated capture aptamer (CAP) and control on the surface, coating the whole gold surface with a functionalization agent (e.g., thiolated protein A), spotting capture antibody (CAb) and control on protein A layer, and finally immobilizing antigen on the same location with that of CAb. Although Luna Labs has demonstrated successful simultaneous RNA and IgG detection using this method, the overall process is lengthy (due to the needed incubation hours and one-at-time spot) and requires a skillset to accurately double spot the antigen on the same site of CAb.

With the CFM, however, the overall process is significantly shortened and simplified. In this technique, the functionalization agent (thiolated protein A) as well as the CAb, antigen, and CAp are all spotted. The general protocol for constructing capture arrays using a CFM is presented in FIG. 8B. By using the two 96-well plates, the capture antibodies, antigen (e.g., ZIKV E, ZIKV Vero E6, or ZIKV COS-1), capture aptamers, and controls were placed in the specific well locations. These plates were then mounted onto the stages of CFM and the instrument was programmed to print the desired spot locations (n=4 for each array). A series of spotting was conducted in the order shown in FIG. 8B: (i) protein A. (ii) capture antibody, CAb, and (iii) antigen plus capture aptamer. In this spotting technique, Luna Labs developed a custom methodology for spotting in which the proteins are printed in a spot-on-spot fashion while the capture aptamer is immobilized in a separate location on a chip. Note that the CFM needle manifold has 48 needles and can create 48 spots for each deployment. No spot is created when there's no sample in the well. After the reagents were placed in the right well locations, the plates were mounted on the stages and the CFM was programmed to spot the desired arrays.

Each immobilization round includes spotting and washing of sample. With a flow rate of 30 μL/min, it took 35 min to complete the protein A immobilization on the gold surface. Note that the time can be shortened by using a higher flow rate. However, selecting the right parameters need to be considered. Previous experiments showed that shorter spotting time and higher flow rate resulted in ineffective capture and detection of the target markers in serum. Three rounds (FIG. 8B (i-iii)) were needed to finish the construction of capture arrays of ZIKV RNA and ZIKV IgG (and/or ZIKV IgM). The process is fully automated, as the CFM was performing all these tasks in a straightforward manner. Aside from being faster, accurate, and automated, the other advantage of CFM over an Arrayer is its capability to casily spot thiolated protein A instead of the typical protein A coating onto the whole gold surface (see FIG. 8Aii versus FIG. 8Bi). This leads to a cleaner CAp spot as it avoids protein A from being in contact with CAp. This method is favorable because it prevents possible interference for RNA detection.

### B. Blocking Components of the Sensing Chip and Assay Validation: ZIKV Indicators in Serum

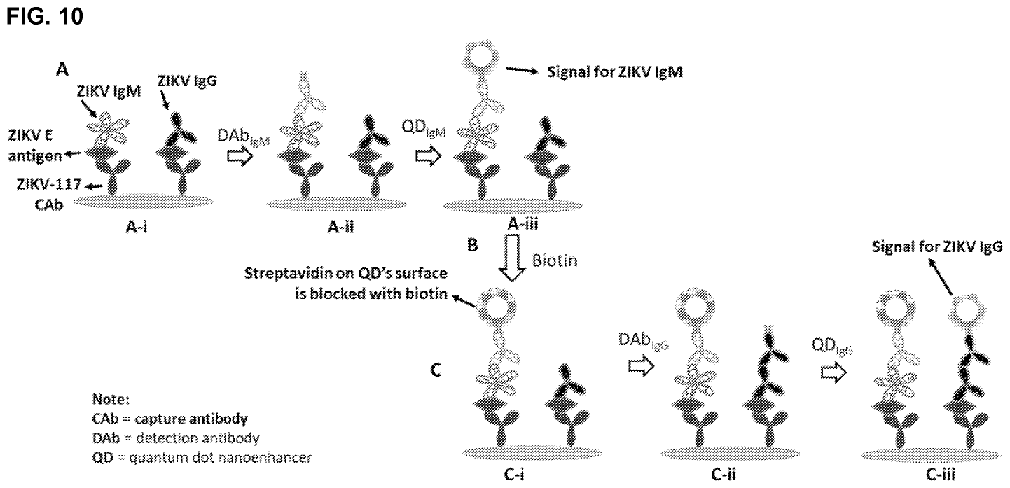

After the chip was spotted with sensing arrays, it was then mounted onto the SPRi instrument and treated with different blocking approaches such as partial, sequential, and mixed described in Table 6. A chip without blocking was also utilized for comparison. Each chip was then analyzed by introducing several injections performed in the order shown in Table 7. This table also lists the purpose for each injection. An overall scheme is presented in FIG. 9 to show the spotting and binding locations while a diagram is shown in FIG. 10 to illustrate the sequential detection of ZIKV antibodies, IgM and IgG.

The MS (multiple spotting) printing was conducted following the general procedure below using Luna Lab's MultiSpot® technology. Using a CFM, capture probes in both nucleic acid (RNA/DNA) and protein (antibodies, antigens) forms are printed simultaneously on the metal surface of a chip. First. thiolated protein A is printed onto a gold film surface, followed by a capture antibody (CAb), and finally an antigen via a spot-on-spot technique on the same location. The final spotting (i.e., antigen) is simultaneously done with capture aptamer (CAP) printed on another specified location of the metal chip surface.

The general printing step is done in order: protein A/CAb/antigen+CAp (see FIG. 8B). However, it is possible to eliminate thiolated protein A, and directly use modified CAb, such as thiolated CAb. It is also feasible to utilize thiolated antigen, bypassing protein A, and CAb spotting.

TABLE 6
Description of blocking techniques implemented on the sensing
chips for multiplex detection. 2 × PBS was used as a diluent.

| Blocking | |
|---|---|
| approach | Blocking Components |
| No blocking | None |
| Partial blocking | 4 mg/mL thiolated methoxy polyethylene glycol |
| | (mPEG, MW of 2,000 g/mol) |
| Sequential | 1^{st} injection: 4 mg/mL thiolated mPEG |
| blocking | (MW of 2,000 g/mol), 2^{nd}: 10 mg/mL mouse IgG, |
| | 3^{rd}: 1% (w/v) BSA |
| Mixed blocking | One injection (all at once): 4 mg/mL thiolated mPEG |
| | (MW of 2,000 g/mol), 10 μg/mL mouse IgG, |
| | 1% (w/v) BSA |

TABLE 7
Series of injections during the analysis of ZIKV RNA, IgM,
and IgG on a chip. A flow rate of 50 μL/min
and a diluent of 2 × PBS buffer were used.

| ID | Injection (in order) | Purpose |
|---|---|---|
| a | 2 × phosphate buffered saline (PBS) | Baseline acquisition |
| b | 0.1 μg/mL rabbit Fc fragment | Final surface blocking |
| c | 1000 ng/mL ZIKV RNA, 1:400 ZIKV | Sample for analysis |
| | IgM, 2.5 μg/mL ZIKV IgG, 1 μL | |
| | RNAse inhibitor spiked in 10% | |
| | human serum | |
| d | 50 nM Biotinylated ZIKV detection | RNA detection |
| | aptamer | |
| e | 10 nM QD_{RNA} | RNA signal amplification |
| f | 10 μg/mL biotinylated anti-human IgM | IgM detection |
| g | 10 nM QD_{IgM} | IgM signal amplification |
| h | 1.5-3 μM biotin | Blocking of QD's |
| | | active site |
| i | 10 μg/mL biotinylated anti-human IgG | IgG detection |
| j | 10 nM QD_{IgG} | IgG signal amplification |

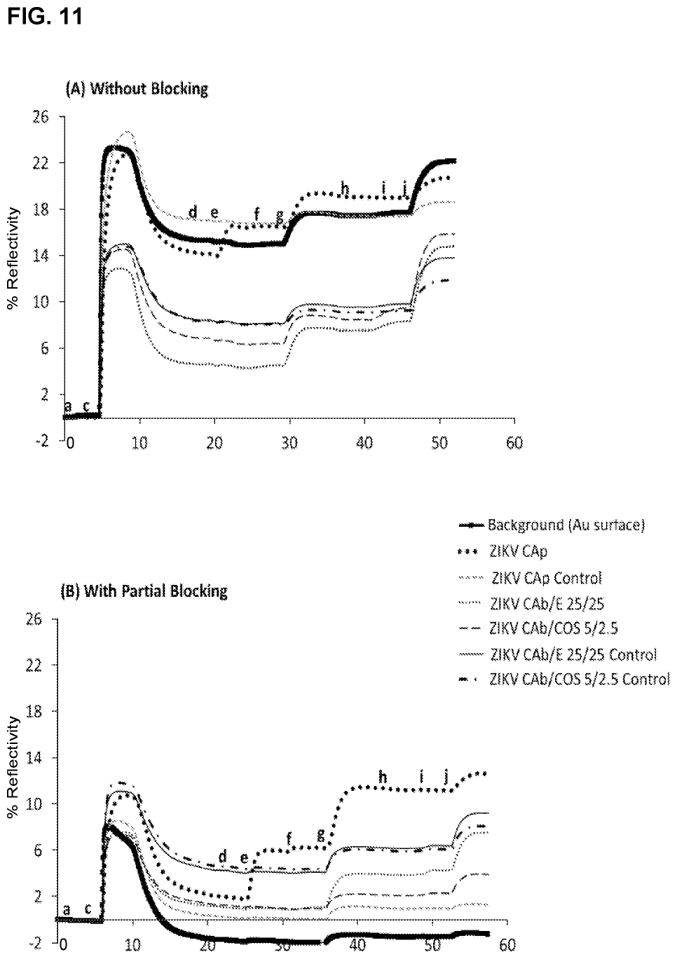

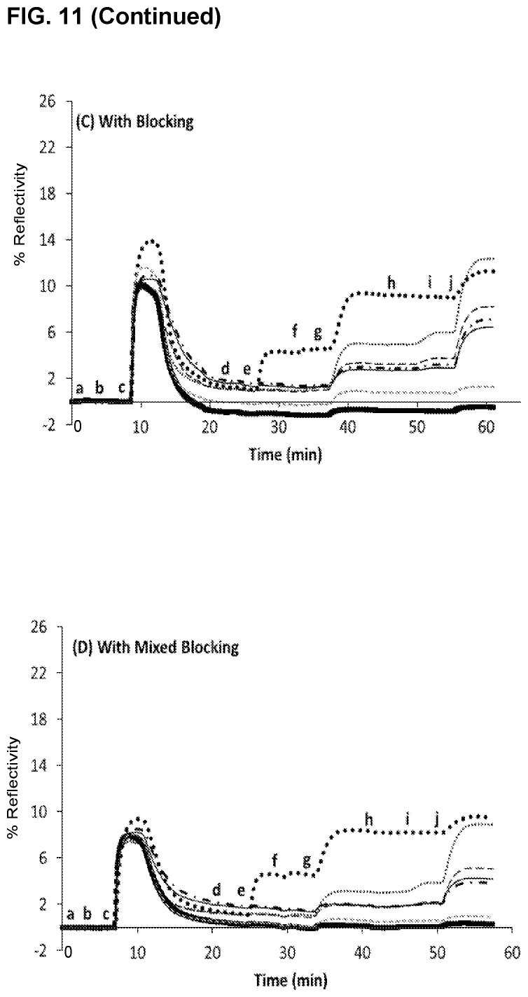

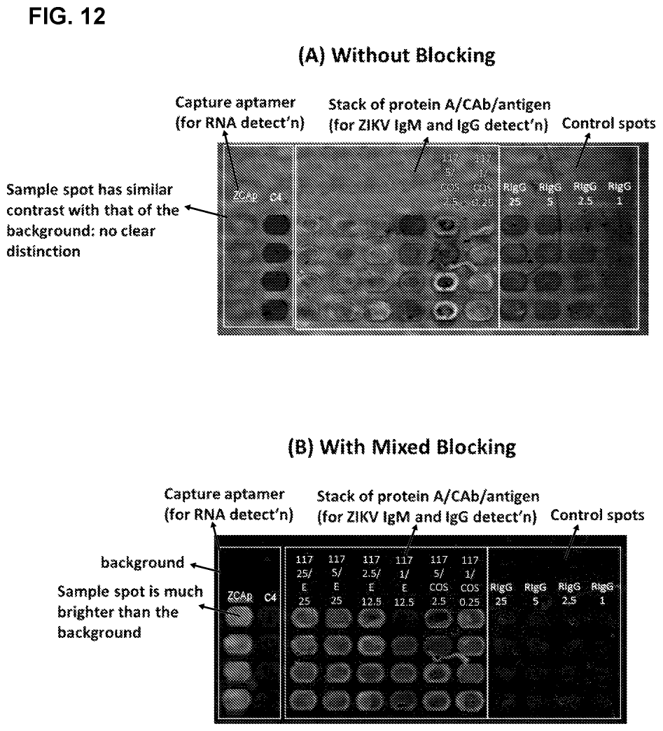

The effectiveness of the blocking was evaluated by the sensorgrams of the background (Au surface only) and the images after QD addition. Thus, for the following assessment, the sensorgram profiles of the gold surface background (thick solid lines in FIGS. 11A-D) and spot images (FIG. 12A and FIG. 12B) were closely monitored.

Without blocking As shown in FIG. 11A, a chip without blocking exhibited a very high background signal (% reflectivity of 22, thick solid line) after serum sample injection and stayed above reflectivity of 15 throughout the analysis. This indicates that the sensing chip was capturing a massive amount of non-specific binding (NSB) molecules from the serum sample. This forms a thick layer on the chip surface. Since SPRi sensing has sensitivity of ˜300 nm from the surface (9), a chip without blocking will not provide accurate detection as SPRi fails to monitor binding events beyond this thickness. All of the capture probes and their controls also exhibited high reflectivity signals (up to 20% reflectivity), hence the detection without blocking the chip surface is not reliable.

Partial blocking FIG. 11B shows the sensorgrams of the background and capture probes for a chip with partial blocking (thiolated mPEG only). Background signal (thick solid line) dramatically decreased below 0 reflectivity indicating successful blocking on the surface, that is, complete elimination of NSB on the gold surface. While capture probes of ZIKV decreased from 10 to 2 reflectivity after serum injection, the control (rabbit IgG) spots remained high (above 5, dash-dotted and thin solid lines) and their reflectivity signals were higher than those of the sample spots. This indicates that the control spots were not sufficiently blocked. The calculation of delta values depends on the signal of the control. Hence, a careful delta value assessment is necessary when implementing a partial blocking as this can lead to erroneous results.

Sequential blocking Sensorgrams for a chip with sequential blocking are presented in FIG. 11C. Background signal exhibited below 0 reflectivity 20 min after serum injection indicating a complete removal of NSB. All of the capture probes decreased from 14 to 0 after serum sample injection and established a baseline at 2 reflectivity starting from 20 min of analysis. This is the correct profile for the analysis in which all spots and background established a baseline closer to the surface allowing accurate monitor of all binding events occurred on the sensing chip as the detection progressed (from ZIKV RNA, IgM, to IgG).

Mixed blocking The blocking approach was further optimized by implementing mixed blocking on the chip surface. The obtained sensorgrams shown in FIG. 11D have similar profiles with those presented in FIG. 11C but this time, the blocking is faster. 95% of NSB elimination was observed (from 15.3 down to 0.77 reflectivity at ˜18 min of analysis). It took an hour to block the chip using a sequential method while only 30 min to treat the surface using mixed blocking. Thus, among the techniques tested, mixed blocking is the most efficient.

FIG. 12A and FIG. 12B show the images of capture probe spots after QD addition on chips that had no treatment and received blocking, respectively. In a chip without blocking, the sample spots have similar contrast with the background hence, detecting the sample true signal is difficult. On the contrary, a chip that was treated with mixed blocking exhibited a very good sample and background contrast. Thus, Luna Labs has successfully demonstrated the importance of proper blocking.

### Example 7. General Protocol for SPRi Analysis Using CFM

The general protocol for SPRi-based detection platform of pathogen indicators in human serum is provided below. It is expected and assumed that an identical or similar protocol can be implemented across multiple other relevant surfaces where functionalization, probe printing, and blocking are required.
1. Proper functionalization requires a clean surface. Wash re-usable, bare, untreated gold chips (from HORIBA Scientific) with ethanol and de-ionized (DI) water and dry with a gentle stream of N₂ gas. Immerse chips in piranha solution with 3:1 (v/v) H₂SO₄: H₂O₂ for 50 min with gentle sonication. Take chips out of the solution, rinse with running DI water for 5 min and ethanol and dry with N₂ stream. Expose chips to UV-Ozone (Jelight Company, Inc.) twice to afford formation of hydrophilic surface.
2. Prepare capture probe layouts on two 96-well plates (Plates 1 and 2) for spotting the sensing arrays using a CFM. First, protein A solution is placed in Plate 1 rows A-D. ZIKV-117 capture antibody (CAb) and control CAb in Plate 2 rows A-D, and ZIKV RNA capture aptamer (CAp), control CAp, and ZIKV E antigen in Plate 2 rows E-H. Using a 48 vertical needle-manifold of CFM, the solutions in the plate will be aspirated to allow successive array spotting of protein A (first spot), followed by a CAb (ZIKV-117, termed as a double spot) and its control, and finally an antigen (ZIKV E, named as a triple spot) together with a CAp and its control (n=4). The resulting arrays will be the one presented in FIG. 8Biii. All of these spots are sequentially printed on the same location via spot-on-spot manner except for the ZIKV RNA CAP which is one-batch spotted on different array site (FIG. 8Biii). Note that the CAp was previously activated using dithiothreitol (DTT) solution. The chip was then placed in a humidity chamber for at least 2 hrs. Negative control lacks binding specificity to the target ZIKV indicators and should not result in observable signal.

Users can establish their own layouts and investigate several probe combinations and concentrations depending on their desired multiple target indicators or biomarkers to be detected. For this type of CFM, a total of 104 various spots can be printed on a single chip. After the reagents are placed in the right well locations, mount the two 96-well plates onto the corresponding stages of CFM. Program the CFM to spot the desired arrays. Use spotting time of 15 min for every batch of spotting at a flow rate of 45 μL/min. The total waiting time to complete the triple printing using these parameters is 1 hr and 45 min. This period can be shortened by lowering the spotting time as well as increasing the flow rate with efficiency that depends on the nature of the capture probes used.

3. Mount the chip onto the SPRi instrument and perform mixed blocking on the surface. Throughout the analysis, use 2×PBS as the running buffer and the solvent for each blocking agent preparation. Prepare 400 μL mixture of 4 mg/mL of thiolated mPEG, 10 μg/mL mouse IgG, and 1% BSA (w/v) and inject this mix into the SPRi flow cell. Wait for 30 min to complete the blocking.

### Example 8. Detection of ZIKV Indicators

Depending on the application, it is desirable that assay methods and systems have one or more of the following characteristics: high throughput, high sensitivity, large dynamic range, high precision and/or accuracy, low cost, low consumption of reagents, compatibility with existing instrumentation for sample handling and processing, short time analysis, multiplexing capability, and applicability to complex sample matrices. Luna Labs's SPRi platform coupled with proprietary SAS possesses most of these attributes.

Luna Labs has demonstrated a number of SAS uses for both single and multiplex detection of various biomarkers (e.g., proteins, engineered antibodies, nucleic acids and extracellular vesicles) in different matrices such as buffers, supernatants, animal serum samples, human serum and clinical serum samples.

In this type of detection, Luna Labs first prepared capture probe layouts on two 96-well plates for spotting the sensing arrays using a CFM. ZIKV capture probe of ZIKV 117 antibody and ZIKV antigens (enveloped E and COS-1) were utilized. Luna Labs implemented various probe combinations and concentrations for analyzing one chip for ZIKV indicator spiked serum sample and another chip for blank (i.e., only serum). 15 min spotting time and 45 μL/min flow rate were the parameters used for spotting.

Subsequent to capture probe spotting, the chip was mounted onto the SPRi instrument and blocked with mixed reagents, one injection of a mixture of 4 mg/mL thiolated mPEG, 10 μg/mL mouse IgG, and 1% w/v BSA. After calibration of the SPRi instrument with high salt concentration (25 mM NaCl solution), the chip was then analyzed using the established SPRi detection protocol. 2×PBS buffer was used as a running buffer and a diluent throughout the experiment.

During the analysis, a series of reagents/sample (see Table 7) was injected to determine the sensorgram for multiplex detection of ZIKV RNA. IgM, and IgG. First, PBS was injected to achieve a background signal. At a stable signal, the final blocking (0.1 μg/mL rabbit Fc fragment) was injected. The ZIKV RNA, IgM, and IgG multiplex analysis was achieved in less than 60 min. Thereafter, a 400 μL sample containing ZIKV RNA (1 μg/mL), RNAse inhibitor (1 μL of 40 U/μL), ZIKV-116 IgG (2.5 μg/mL), and ZIKV IgM (1:4 dilution) spiked in 10% serum was injected. These indicators were captured by their corresponding probes immobilized on the surface and were subsequently detected. A 3-step sequential detection was implemented: (1) Detection of ZIKV RNA was done by injecting 50 nM ZIKV biotinylated detection aptamer (DAp), followed by the nanoenhancer solution, quantum dot QDRNA for signal amplification. (2) Detection of ZIKV IgM was carried out by injecting 10 μg/mL biotinylated anti-human IgM followed by QD_{IgM}. Prior to the last detection, a 3 mM biotin was introduced in the flow to block the active site of the previously injected streptavidin-coated QD_{RNA} and QD_{IgM}. (3) Detection of ZIKV IgG was then conducted by injecting 2.5 μg/mL biotinylated anti-human IgM followed by QD_{IgG}. All QD solutions were prepared at a concentration of 10 nM. For each indicator detection, delta value (i.e., control-subtracted reflectivity signal) was calculated as follows:

Δvalue=[Δ% reflectivity of sample(After QD−Before QD)]−[Δ% reflectivity of control(After QD−Before QD)

Since both spiked sample and blank were analyzed, a delta value ratio (DVR) was also calculated. DVR is the ratio of signal reflectivities obtained in the sample and the blank.

By using the mixed blocking protocol, Luna Labs analyzed both a spiked serum sample and a blank. The obtained delta values were then plotted against the various probe concentrations implemented in the study. Majority of the probes (8 out of 13, data not shown) indicates positive detection of ZIKV RNA, IgM, and IgG indicators in spiked serum samples using Luna Labs's functionalization, printing, and blocking technologies.

### Example 9. Detection of a Biomarker: Traumatic Brain Injury Biomarker

Luna Labs has also demonstrated the analysis of a traumatic brain injury (TBI) biomarker, CCL11 spiked in 10% human serum sample. CCL11 is a protein associated with age-associated cognitive decline. Recent study revealed that it is also dominant in the brain and cerebrospinal fluid in chronic traumatic encephalopathy (CTE) disease (10).

Analysis of CCL11 was carried out using the established SAS protocol for surface activation (see, e.g., Table 5). Results (data not shown) indicate that the injection of a control sample (10% human serum only) did not show any signal on the sensing chip after QD addition. Very subtle spot signals appeared from lower biomarker concentrations (0.5 and 1 ng/mL.) while visible and well-defined spots were observed for higher concentrations (5 and 10 ng/ml). 10 ng/mL CCL11 in 10% serum exhibited appreciable signal with a delta (Δ) value of 3.0 after QD. Overall, cotaxin CCL11 brain injury biomarker was successfully detected using Luna Labs's SPRi/SAS platform with lowest concentration identified at 1 ng/ml in 10% human serum. Note that the TBI-related biomarker serum levels are in the range 1.5 ng/ml-22 ng/ml (11).

### Example 10. Detection of Multiple Protein Biomarkers: Organ Injury Biomarkers

The same SAS protocol was implemented for the simultaneous detection of 6 protein biomarkers: 3 inflammatory biomarkers (IL1b, MIP1b and MIP3) and 3 acute lung injury biomarkers (TNF-α IL1b and IL8) (12) spiked in 10% human serum.

Results (data not shown) indicate that injection of control sample (human serum only) did not show any signal on the sensing chip after QD addition. Spot signals only appeared from samples containing 6 biomarkers spiked in 10% human serum. Note that each biomarker has different binding affinity to its corresponding detection antibody. Thus, different reflectivity values were observed.

### Example 11. Detection of Multiple Protein Biomarkers and Determination of KD: Atopic Dermatitis Biomarkers

Luna Labs has also developed a nanoenhanced Surface Plasmon Resonance imaging-based sparing assay (nanoSPRISA™) for rapid monitoring, multiplex detection, and assessment of cytokines associated with atopic dermatitis (AD) using limited amount of biological samples. Luna Labs has demonstrated the multiplex detection of 4 different cytokines associated with AD: IL13, IL18. IL4 and MIP4 (13). The resulting assay was termed. These cytokines (10 ng/mL each) were spiked in 10% human serum. Following the same SAS protocol on the surface, simultaneous analysis was carried out.

Among the cytokines, MIP4 reveals the highest signal (brightest spot image) while IL18 gives the lowest signal (data not shown). This is attributed to their binding strengths with MIP4 having the strongest and IL18 the weakest cytokine/antibody interaction.

Binding strength/affinity of cytokines to their respective antibodies is an important factor when determining the smallest clinical sample volume possible to be used for the SPRi-based assay. It is related to a kinetic parameter K_{D}, the ratio of dissociation to association constants (k_{d}/kₐ) of the ligand-protein interaction (14). The lower the K_{D} value, the higher the affinity of the antibody to its antigen.

To demonstrate the applicability of SAS in determining the KD value of antibody/antigen binding interaction, SAS was applied on the surface of the chip. The SAS was performed using the procedure below. The functionalized and spotted chip was mounted onto the SPRi instrument and coated with blocking agents in an orderly fashion to effectively prevent unwanted human serum constituents (i.e., non-specific binding proteins) from adhering onto the surface. The blocking system consists of the thiolated mPEG (mPEG-SH. 4 mg/mL with molecular weight 2,000 g/mol), the human: rabbit IgG mix (1:1 molar ratio, 10 μg/mL each), the bovine serum albumin (BSA, 1% wt/v), and the human Fc fragment (0.5 μg/mL). Diluent was 1×PBS.

Analysis was then conducted by injecting 1×PBS buffer to obtain a baseline signal. Single injection of IL13 was introduced in the flow cell at a running buffer flow rate of 20 μL/min to allow effective capture of IL13 by immobilized anti-IL13 spots on the chip surface. The obtained sensorgrams were then globally fit to a 1:1 biomolecular interaction model (software: SrubberGen, HORIBA Scientific) to calculate binding kinetic parameters: kₐ, k_{d} and K_{D}. This protocol was repeated for the binding strength analyses of MIP4 and IL18 with their corresponding antibodies.

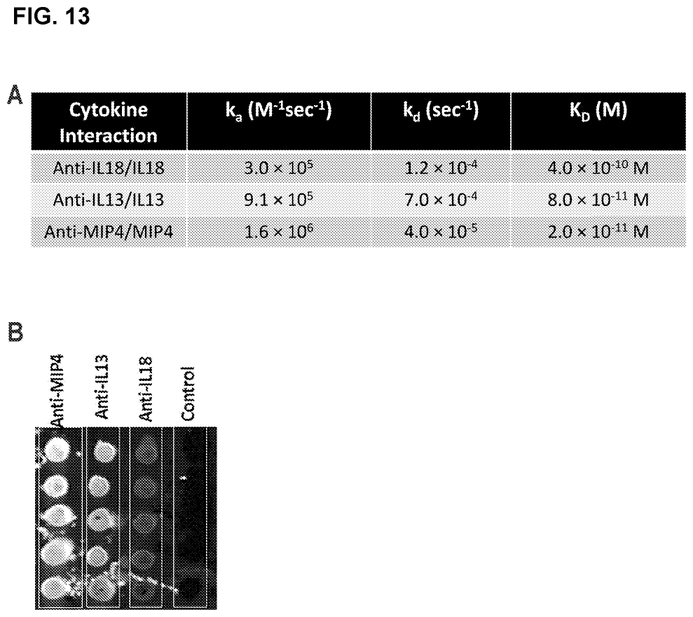

Among the three cytokines (FIG. 13A), MIP4 has the highest binding affinity to its capture antibody (anti-MIP4/MIP4 K_{D})=2×10⁻¹¹ M), followed by IL13 (anti-IL13/IL13 K_{D})=8×10⁻¹¹ M), and then IL18 (anti-IL18/IL18 KD)=4×10⁻¹⁰ M). The calculated K_{D} value of IL18 coincides very well with the value (K_{D}=3.78×10⁻¹⁰ M) obtained by other researchers (15). The binding strength trend (MIP4>IL13 >IL18) is also consistent with the signals obtained from the multiplex detection of MIP4, IL13 and IL18 in buffer (FIG. 13B) and in 10% human serum (data not shown). The strongest anti-MIP/MIP4 coupling results in brightest spot image and highest signal among cytokines. These results demonstrate that by applying SAS on the surface, binding affinity of proteins are successfully assessed.

Luna Labs also performed multiplex detection of cytokines in unknown clinical serum samples from AD patients following the same protocol used for cytokine-spiked serum sample analysis. Results (data not shown) indicate that high levels of MIP4 and IL18 cytokines in AD serum samples with concentration increases with AD severities. The trend of MIP4 levels is similar with existing literature where researchers used ELISA to detect MIP4 from patients with various AD severities (16). Generally, IL4 is hard to observe in AD sample (concentration ˜0 ng/mL, data not shown). IL13 can be present if the sample is from a patient with extremely severe condition. MIP4 and IL18 are in very good correlations with various AD severities via EASI score (data not shown).

The value of Luna Labs's nanoSPRiSA is to monitor the immune status of patients following multiple cytokines to allow effective treatment of AD. By detecting a panel of AD biomarkers, Luna Labs can aid in treatment selection and proper diagnosis distinguishing AD from other dermatological pathologies such as contact dermatitis and psoriasis that can appear to be AD but require alternative treatments.

nanoSPRiSA is a multiplex detection of a panel of cytokines and is able to determine the levels of potential markers for AD. With multiplexing capability. Luna Labs's nanoSPRi platform can give a snapshot of a patient's status at a single point in time and provide high diagnostic accuracy and evidence on AD patients. The identified cytokine concentrations are associated with various severities of AD. With this kind of information, doctors can identify the patient's status, predict flare-ups, and determine how well the patient is responding to the given drugs/treatment. By monitoring the cytokines, clinicians know if they need to adjust treatment strategies like prescribing different or additional medicines to minimize the severity of a patient's symptoms in a pre-emptive nature, thus improving patient care.

### Example 12. Detection of a Nucleic Acid: Detection of DNA-Based Liver Injury Biomarkers in Serum Samples

Luna Labs has demonstrated the capability of SAS to be used for the detection of smaller molecules such as DNA-based biomarkers in serum. Results (data not shown) indicate that successful detection of 42-nucleotide (nt) long single-stranded DmiR122 DNA biomarker in 10% serum with high S/N ratio was achieved. DmiR122 is a DNA counterpart of microRNA122, a biomarker for liver injury (17,18). DNA aptamer probes were used to capture and detect DmiR122.

Luna Labs has also performed simultaneous detection of DmiR122 with ANGPTL3. ANGPTL3 (human angiopoietin-like 3), encodes a member of a family of secreted proteins that are expressed predominantly in the liver, and is important for lipid metabolism and angiogenesis (19.20). Results (data not shown) indicate that control spots exhibited flat profile (Δ% reflectivity=0) and dark image indicating negligible binding of any of biomarkers with the control aptamers. Similarly, background signal revealed 0% reflectivity and black image signifying that the sensing chip surface was completely blocked by Luna Labs's blocking mix in SAS resulting in high S/B ratio of DmiR 122 and ANGPTL3 biomarkers.

Luna Labs demonstrated the use of SAS in detecting even shorter sequence nucleic acids, such as microRNAs which typically consist of only 21 to 23 nucleotides (nt). In general, hybridization of a short strand (≤23-nt long) with its complementary aptamer is weak, making the detection technically challenging. To circumvent this problem, Luna Labs strategically designed and incorporated locked nucleic acid (LNA) probe in the original capture and detection aptamer sequence design to increase melting temperature and hence strengthen aptamer binding with DNA/RNA targets of shorter sequence. By using SAS and a transition from DNA to LNA aptamers. Luna Labs has also demonstrated a robust detection of 23-nt long DNA-based liver injury biomarker in serum with high S/N ratio (data not shown).

### Example 13. Detection of Nucleic Acid and Protein on Same Chip

To utilize the full potential of SAS in preventing non-specific binding proteins in human serum. Luna Labs integrated the design for simultaneous analysis of protein (IL4 as a representative) and nucleic acid (DmiR122) biomarkers on the sensing chip surface.

To do this, thiolated capture LNA aptamer was first spotted onto the gold thin film. Thereafter, thiolated protein A was coated on the chip using a press load. Anti-IL4 capture antibody was spotted onto the chip which was subsequently blocked by a blocking mix in SAS. DmiR122 and IL4 biomarkers in 10% serum were injected followed by Fc fragment, detection mix (consisting of LNA aptamer and detection IL4 antibody) and finally the nanoenhancer QD. In this specific example, the blocking system consisted of thiolated mPEG (4 mg/mL with molecular weight of 2,000 g/mol), IgG mix (1:1 molar ratio human IgG: rabit IgG, 10 μg/mL each), bovine serum albumin (BSA. 1% wt/v) and Fc fragment (0.5 u g/mL). Diluent was 2×PBS.

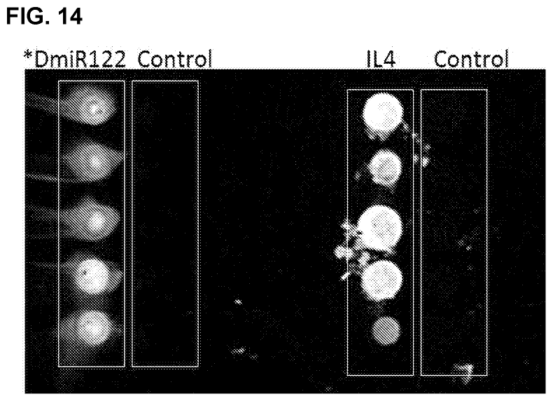

By using this method, DmiR122 and IL4 signals were visible after QD addition (FIG. 14) indicating successful detection of these biomarkers in serum sample on a single chip.

### Example 14. Detection of Purified Exosome Vesicles in Phosphate Buffered Saline

In the quest to determine extracellular vesicles (EV) associated with cancer, Luna Labs has used SAS to prevent the surface from binding with unwanted proteins on the exosome surface. Luna Labs has successfully detected CD63 and CD9, the general markers on the surface of EV (data not shown). This platform is designed to detect other EV-derived biomarkers (such as glypican-1, tetraspanin 8, and CD44v6) in serum samples from patients with pancreatic cancer (21,22).

### Example 15. Detection of Ebola and Dengue Gene-Encoded Monoclonal Antibodies in Purified Supernatants

By using the established SAS protocol (see e.g., Table 5), Luna Labs has also carried out multiplex detection of two gene-encoded monoclonal antibody (DMAb) types: Ebola DMAb11 and Dengue DVSF1 in purified supernatant samples (23.24). Obtained sensorgrams (data not shown) indicate successful detection of Ebola and Dengue DMAbs. Luna Labs is confident that this established protocol can be extended to detect antigen, antibody isotypes and genomic DNA/RNA indicators from different pathogens (e.g., Zika virus, Dengue virus, many others.).

### Example 16. Detection of Methylated Genes Associated with Post-Traumatic Stress Disorder (PTSD)

Luna Labs also successfully demonstrated the proof-of-concept for the assay's multiplexing capability of detecting methylated genes associated with PTSD. First, Luna Labs demonstrated the simultaneous detection of 2 different PTSD bases from 2 target methylated genes (data not shown). Detection of a PTSD base using the EpiNanoSPRi platform was carried out as follows: Briefly, the gold sensing chip surface was first spotted with dithiothreitol (DTT)-activated capture aptamer (CAp or PTSD site probe, see sequences below) of different concentrations (50, 100, 200, and 400 μg/mL) and incubated for 1 hr in a humidity chamber (65-75% relative humidity). A control CAp (with a guanine base at the PTSD site, 100 μg/mL) was spotted as well. The sensing chip was then washed with de-ionized water (DI), dried with gentle stream of N₂ and mounted onto the SPRi instrument.

The chip was then blocked in-line with thiolated methoxypolyethylene glycol (MW of 2,000) by injecting 4 mg/mL of thiolated mPEG in the flow cell with running buffer flow rate of 50 μL/min. This in-line coating was done to obtain monolayer mPEG assembly on chip surface. Prior to analysis, the instrument was calibrated with high salt concentration, 25 mM NaCl. Throughout the experiment, the running buffer of HEPES-EP containing 10 mM HEPES, 150 mM NaCl, 3 mM EDTA and 0.005% Tween20 surfactant, pH7.4 was utilized.

The methylated AHRR34 gene analysis was carried out as follows: HEPES-EP buffer was first injected in the flow cell to provide the baseline signal. After the signal was stabilized, 1000 ng/ml of methylated AHRR34 spiked in hybridization buffer, 3×SSPE (sodium chloride-sodium phosphate-EDTA, 0.2 M phosphate buffer, 2.98 M NaCl, 20 mM EDTA, pH7.4) was injected allowing target DNA to hybridize with the immobilized Cap, PTSD site probe, array. Thereafter, 3 μg/mL biotinylated DAp in in 2×PBS containing 0.05% (v/v) Tween 20 and 0.1% (w/v) BSA was injected at a slow flow rate of 20 μL/min to allow effective binding of anti-5mC to 5mC, PTSD base. After approximately 15 min, the flow rate was returned to the typical 50 μL/min rate and injection of 10 nM streptavidin-coated QD was performed for signal amplification.

Luna Labs then extended the number of targets analyzed and achieved multiplex detection for 5 PTSD bases from 5 synthetic methylated genes and genomic DNA (data not shown).

These results show a definitive demonstration of a rapid, high throughput, simplified assay platform that can monitor a panel of PTSD bases in target genes within an hour using Luna Labs's SAS technology and SPRi-based platform. This detection platform could potentially be extended to whole blood from PTSD patients to monitor target PTSD bases.

Table 8 shows the list of representative use cases that utilized Luna Labs's surface functionalization, blocking and printing techniques for the successful detections of different types of biomarkers using SPRi-based platform.

TABLE 8
Summary of representative use cases for successful detections
of various biomarkers using Luna Labs's surface functionalization,
blocking, and printing technologies.

| Biomarker | Biomarker of | | Associated |
|---|---|---|---|
| Type | Interest | Matrix | Disease |
| Antibodies, and | ZIKV RNA, IgM, | Serum | Zika virus |
| nucleic acid | and IgG | | |
| (RNA) | | | |
| Antigen | CCL11 | Serum | Traumatic |
| | | | brain injury |
| Antigens | IL1b, MIP1b, MIP3, | Serum | Organ (acute |
| | TNF-α IL1b, | | lung and liver) |
| | and IL8 | | injuries |
| Antigens | IL4, IL13, IL18, | Serum, and | Atopic |
| | and MIP4 (or | clinical | dermatitis |
| | CCL18) | serum samples | |
| Nucleic acids | DNA counterpart of | Serum | Liver injury |
| (DNA) | miR122 (DmiR22), | | |
| | and ANGPTL3 | | |
| | (human angiopoietin- | | |
| | like 3) | | |
| Protein and | IL4 and DmiR122 | Serum | Inflammatory |
| nucleic | | | and lung |
| acid (DNA) | | | injury |
| Exosomes or | CD9, CD63 | Buffer | Pancreatic |
| extracellular | | | cancer |
| vesicles | | | |
| Gene-encoded | DMAb-11, and | Supernatant | Ebola virus |
| monoclonal | DVSF1 | | and Dengue |
| antibody | | | virus |
| Human cell | 5-methylcytosine | Buffer | Post-traumatic |
| line genomic | bases in AHRR, | | stress disorder |
| DNA | MAD1L1, FKBP5 | | (PTSD) |
| | genes | | |

TABLE 9
Exemplary Surface Agent Amino Acid Sequences

| Identity | Sequence |
|---|---|
| Protein A | AAQHDEAQQNAFYQVLNMPNLNADQRNGFIQSLKDDPSQSANVLGEAKKLNDSQAPKAD |
| | AQQNNFNKDQQSAFYEILNMPNLNEAQRNGFIQSLKDDPSQSTNVLGEAKKLNESQAPK |
| | ADNNFNKEQQNAFYEILNMPNLNEEQRNGFIQSLKDDPSQSANLLSEAKKLNESQAPKA |
| | DNKFNKEQQNAFYEILHLPNLNEEQQNAFYEILHLPNLTEEQRNGFIQSLKDDPSVSKE |
| | ILAEAKKLNDAQAP (SEQ ID NO: 1) |
| Protein A | AQHDEAQQNAFYQVLNMPNLNADQRNGFIQSLKDDPSQSANVLGEAQKLNDSQAP SEQ |
| Ig-binding | ID NO: 2) |
| domain | |
| Protein A | ADAQQNNFNKDQQSAFYEILNMPNLNEAQRNGFIQSLKDDPSQSTNVLGEAKKLNESQA |
| Ig-binding | PSLKDDPSQSTNVLGEAKKLNESQAP (SEQ ID NO: 3) |
| domain | |
| Protein A | ADNNFNKEQQNAFYEILNMPNLNEEQRNGFIQSLKDDPSQSANLLSEAKKLNESQAP |
| Ig-binding | MPNLNEEQRNGFIQSLKDDPSQSANLLSEAKKLNESQAP (SEQ ID NO: 4) |
| domain | |
| Protein A | ADNKFNKEQQNAFYEILHLPNLNEEQRNGFIQSLKDDPSQSANLLAEAKKLNDAQAP |
| Ig-binding | (SEQ ID NO: 5) |
| domain | |
| Protein A | ADNKFNKEQQNAFYEILHLPNLTEEQRNGFIQSLKDDPSVSKEILAEAKKLNDAQAP |
| Ig-binding | (SEQ ID NO: 6) |
| domain | |
| Protein A | VDNKFNKEQQNAFYEILHLPNLNEEQRNAFIQSLKDDPSQSANLLAEAKKLNDAQAPAN |
| Ig-binding | LLAEAKKLNDAQAP (SEQ ID NO: 7) |
| domain | |
| Reduced | FNMQQQRRFYEALHDPNLNEEQRNAKIKSIRDD (SEQ ID NO: 8) |
| Z domain | |
| Protein G | GETTTEAVDAATAEKVFKQVANDNGVDGEWTYDDATKTFTVTEKPEVIDASELTPAVTTYKLVI |
| | NGKTLKGETTTEAVDAATAEKVFKQYANDNGVDGEWTYDDATKTFTVTEKPEVIDASELTPAVT |
| | TYKLVINGKTLKGETTTKAVDAETAEKAFKQYANDNGVDGVWTYDDATKTFTVTE |
| | (SEQ ID NO: 9) |
| Protein G | GETTTEAVDAATAEKVFKQYANDNGVDGEWTYDDATKTFTVTEK (SEQ ID NO: 10) |
| Ig-binding | |
| domain | |
| Protein G | ELTPAVTTYKLVINGKTLKGETTTEAVDAATAEKVFKQYANDNGVDGEWTYDDATKTFT |
| Ig-binding | VTE (SEQ ID NO: 11) |
| domain | |
| Protein G | ELTPAVTTYKLVINGKTLKGETTTKAVDAETAEKAFKQYANDNGVDGVWTYDDATKTFT |
| Ig-binding | VTE (SEQ ID NO: 12) |
| domain | |
| Protein L | KEETPETPETDSEEEVTIKANLIFANGSTQTAEFKGTFEKATSEAYAYADTLKKDNGEY |
| | TVDVADKGYTLNIKFAGKEKTPEEPKEEVTIKANLIYADGKTQTAEFKGTFEEATAEAY |
| | RYADALKKDNGEYTVDVADKGYTLNIKFAGKEKTPEEPKEEVTIKANLIYADGKTQTAE |
| | FKGTFEEATAEAYRYADLLAKENGKYTVDVADKGYTLNIKFAGKEKTPEEPKEEVTIKA |
| | NLIYADGKTQTAEFKGTFAEATAEAYRYADLLAKENGKYTADLEDGGYTINIRFAG |
| | (SEQ ID NO: 13) |
| Protein L | KEETPETPETDSEEEVTIKANLIFANGSTQTAEFKGTFEKATSEAYAYADTLKKDNGEY |
| Ig-binding | TVDVADKGYTLNIKFAG (SEQ ID NO: 14) |
| domain | |
| Protein L | KEKTPEEPKEEVTIKANLIYADGKTQTAEFKGTFEEATAEAYRYADALKKDNGEYTVDV |
| Ig-binding | ADKGYTLNIKFAG (SEQ ID NO: 15) |
| domain | |
| Protein L | KEKTPEEPKEEVTIKANLIYADGKTQTAEFKGTFEEATAEAYRYADLLAKENGKYTVDV |
| Ig-binding | ADKGYTLNIKFAG (SEQ ID NO: 16) |
| domain | |
| Protein L | KEKTPEEPKEEVTIKANLIYADGKTQTAEFKGTFAEATAEAYRYADLLAKENGKYTADL |
| Ig-binding | EDGGYTINIRFAG (SEQ ID NO: 17) |
| domain | |
| Ig-binding | WQRHGI (SEQ ID NO: 18) |
| peptide | |
| Ig-binding | MWRGWQ (SEQ ID NO: 19) |
| peptide | |
| Ig-binding | RHLGWF (SEQ ID NO: 20) |
| peptide | |
| Ig-binding | GWLHQR (SEQ ID NO: 21) |
| peptide | |
| Ig-binding | EPIHRSTLTALL (SEQ ID NO: 22) |
| peptide | |
| Ig-binding | HWRGWV (SEQ ID NO: 23) |
| peptide | |
| Ig-binding | HYFKFD (SEQ ID NO: 24) |
| peptide | |
| Ig-binding | HFRRHL (SEQ ID NO: 25) |
| peptide | |
| Ig-binding | HWCGWV (SEQ ID NO: 26) |
| peptide | |
| Ig-binding | RWHYFK (SEQ ID NO: 27) |
| peptide | |
| Ig-binding | WFRHYK (SEQ ID NO: 28) |
| peptide | |
| Ig-binding | NKFRGKYK (SEQ ID NO: 29) |
| peptide | |
| Ig-binding | NARKFYKG (SEQ ID NO: 30) |
| peptide | |
| Ig-binding | FYWHCLDE (SEQ ID NO: 31) |
| peptide | |
| Ig-binding | FYCHWALE (SEQ ID NO: 32) |
| peptide | |
| Ig-binding | FYCHTIDE (SEQ ID NO: 33) |
| peptide | |
| Ig-binding | RRGW (SEQ ID NO: 34) |
| peptide | |
| Ig-binding | KHRFNKD (SEQ ID NO: 35) |
| peptide | |
| Ig-binding | RWHYFK (SEQ ID NO: 36) |
| peptide | |
| Ig-binding | DCAWHLGELVWCT (SEQ ID NO: 37) |
| peptide | |
| Ig-binding | TWKTSRISIF (SEQ ID NO: 38) |
| peptide | |
| Streptavidin | DPSKDSKAQVSAAEAGITGTWYNQLGSTFIVTAGADGALTGTYESAVGNAESRYVLTGR |
| | YDSAPATDGSGTALGWTVAWKNNYRNAHSATTWSGQYVGGAEARINTQWLLTSGTTEAN |
| | AWKSTLVGHDTFTKVKPSAASIDAAKKAGVNNGNPLDAVQQ (SEQ ID NO: 39) |
| Streptavidin | EAGITGTWYNQLGSTFIVTAGADGALTGTYESAVGNAESRYVLTGRYDSAPATDGSGTA |
| fragment | LGWTVAWKNNYRNAHSATTWSGQYVGGAEARINTQWLLISGTTEANAWKSTLVGHDTFT |
| | KVKPSAAS (SEQ ID NO: 40) |
| Avidin | ARKCSLTGKWTNDLGSNMTIGAVNSRGEFTGTYITAVTATSNEIKESPLHGTQNTINKR |
| | TQPTFGFTVNWKFSESTTVFTGQCFIDRNGKEVLKTMWLLRSSVNDIGDDWKATRVGIN |
| | IFTRLRTQKE (SEQ ID NO: 41) |
| Tamavidin | MSDVQSSLTGTWYNELNSKMELTANKDGTLTGKYLSKVGDVYVPYPLSGRYNLQPPAGQ |
| 2 | GVALGWAVSWENSKIHSATTWSGQFFSESSPVILTQWLLSSSTARGDVWESTLVGNDSF |
| | TKTAPTEQQIAHAQLHCRAPRLK (SEQ ID NO: 42) |
| Tamavidin | MSDVQSSLTGTWYNELNSKMELTANKDGTLTGKYLAKVGDVYVPYPLSGRYNLQPPAGQ |
| 2-S36A | GVALGWAVSWENSKIHSATTWSGQFFSESSPVILTQWLLSSSTARGDVWESTLVGNDSF |
| | TKTAPTEQQIAHAQLHCRAPRLK (SEQ ID NO: 43) |

### REFERENCES

1. Amsbio. Immunoassay blocking agents: A practical guide. World wide web: amsbio.com pp 1-16.
2. Nicoya Life Sciences. Reducing non-specific binding in surface plasmon resonance experiments. World wide web:.nicoyalife.com; World wide web:.nicoyalife.com/wp-content/uploads/2015/11/Reducing-Non-Specific-Binding-in-Surface-Plasmon-Resonance-Experiments.pdf pp 1-9.
3. Buchwalow I, Samoilova V, Boecker W, & Tiemann M (2011) Non-specific binding of antibodies in immunohistochemistry: fallacies and facts. Scientific Reports 1:28.
4. Contreras-Naranjo JE & Aguilar O (2019) Suppressing Non-Specific Binding of Proteins onto Electrode Surfaces in the Development of Electrochemical Immunosensors. Biosensors 9 (1).
5. Malic L, Sandros MG, & Tabrizian M (2011) Designed biointerface using near-infrared quantum dots for ultrasensitive surface plasmon resonance imaging biosensors. Analytical Chemistry 83 (13): 5222-5229.
6. Vance S, Zeidan E, Henrich VC, & Sandros MG (2016) Comparative Analysis of Human Growth Hormone in Serum Using SPRi, Nano-SPRi and ELISA Assays. Journal of Visualized Experiments (107).
7. Zeidan E, Li S, Zhou Z, Miller J, & Sandros MG (2016) Single-multiplex detection of rrgan injury biomarkers using SPRi based nano-immunosensor Scientific Reports 6 (36348): 1-8.
8. Reichert Technologies. Explained: Sensor chips for surface plasmon resonance and other applications. https://bitesizebio.com/34644/biosensor-chips-surface-plasmon-resonance/.
9. Zeidan E, Li S, Zhou Z, Miller J & Sandros MG (2016) Single-multiplex detection of organ injury biomarkers using SPRi based nano-immunosensor. Scientific Reports 6:36348.
10. Cherry J D, et al. (2017) CCL11 is increased in the CNS in chronic traumatic encephalopathy but not in Alzheimer's disease. Plos One 12 (9): e0185541.
11. Dash P K, Zhao J, Hergenroeder G, & Moore AN (2010) Biomarkers for the diagnosis, prognosis, and evaluation of treatment efficacy for traumatic brain injury. Neurotherapeutics: The Journal of the American Society for Experimental NeuroTherapeutics 7 (1): 100-114.
12. Barnett N & Ware L B (2011) Biomarkers in acute lung injury-Marking forward progress. Critical Care Clinics 27 (3): 661-683.
13. Bin L & Leung D Y (2016) Genetic and epigenetic studies of atopic dermatitis. Allergy, Asthma, and Clinical Immunology: Official Journal of the Canadian Society of Allergy and Clinical Immunology 12:52.
14. Kamat V & Rafique A (2017) Exploring sensitivity & throughput of a parallel flow SPRi biosensor for characterization of antibody-antigen interaction. Analytical Biochemistry 525:8-22.
15. Bio-Rad (2006) Rapid and detailed analysis of multiple antigen-antibody pairs using the ProteOn™ XPR36 protein interaction array system. Bio-Rad Bulletin Tech Note 5360:1-4.
16. Gunther C, et al. (2005) CCL18 is expressed in atopic dermatitis and mediates skin homing of human memory T cells. Journal of Immunology 174 (3): 1723-1728.
17. Bandiera S, Pfeffer S, Baumert T F, & Zeisel MB (2015) miR-122-A key factor and therapeutic target in liver disease. Journal of Hepatology 62 (2): 448-457
18. Coulouarn C. Factor V M, Andersen J B, Durkin M E, & Thorgeirsson SS (2009) Loss of miR-122 expression in liver cancer correlates with suppression of the hepatic phenotype and gain of metastatic properties. Oncogene 28 (40): 3526-3536.
19. Conklin D, et al. (1999) Identification of a mammalian angiopoietin-related protein expressed specifically in liver. Genomics 62 (3): 477-482.
20. Kersten S (2017) Angiopoietin-like 3 in lipoprotein metabolism. Nature Reviews. Endocrinology 13 (12): 731-739.
21. Melo S A, et al. (2015) Glypican-1 identifies cancer exosomes and detects early pancreatic cancer. Nature 523 (7559): 177-182.
22. Wang H, Rana S, Giese N, Buchler M W, & Zoller M (2013) Tspan8, CD44v6 and alpha6beta4 are biomarkers of migrating pancreatic cancer-initiating cells. International Journal of Cancer 133 (2): 416-426.
23. Muralidhara B K, et al. (2016) Critical considerations for developing nucleic acid macromolecule based drug products. Drug Discovery Today 21 (3): 430-444.
24. Flingai S, et al. (2015) Protection against dengue disease by synthetic nucleic acid antibody prophylaxis/immunotherapy. Scientific Reports 5:12616.

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus. all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the ordinary skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the ordinarily skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A method of preparing a substrate having an inert metal surface, comprising:
a) functionalizing the inert metal surface with a surface agent to produce a surface-agent-functionalized surface; and
b) blocking the inert metal surface to reduce or prevent non-specific binding.

2. The method of claim 1, wherein the inert metal surface is a gold surface, a silver surface, or a gold/silver alloy surface coated on the substrate.

3. The method of claim 1 or 2, wherein the surface agent is a protein, preferably, protein A, protein G, protein L, avidin, or streptavidin.

4. The method of claim 1 or 2, wherein the surface agent is protein A modified with amine, carboxyl, hydroxyl, and/or thiol.

5. The method of claim 1 or 2, wherein the surface agent is a thiolated protein A, wherein the protein A is modified with a thiol.

6. The method of any of claims 1-5, wherein the functionalizing step a) comprises treating the inert metal surface with a solution containing the surface agent at a concentration of about 5 μg/mL to about 50 μg/mL with a press load, preferably, the press load is a ceramic, glass or polymer load, which is applied to the substrate to result a stress from about 3-30 Pa.

7. The method of any of claims 1-5, wherein the functionalizing step a) comprises treating a first area of the inert metal surface with a solution containing the surface agent at a concentration of about 0.1 μg/mL to about 15 μg/mL, e.g., using a CFM.

8. The method of claim 7, further comprising treating a second area of the inert metal surface with a surface-agent-independent capture molecule, wherein the second area is different from the first area, wherein the surface-agent-independent capture molecule is capable of directly or indirectly binding to the inert metal surface without binding to the surface agent.

9. The method of any of claims 1-8, wherein the blocking step b) comprises treating the surface-agent-functionalized surface with a first blocking solution comprising a modified polyethylene glycol (PEG), wherein the modified PEG is modified with an amine, carboxyl, or thiol at one end.

10. The method of claim 9, wherein the modified PEG is a thiolated PEG, wherein the PEG is modified with a thiol at one end.

11. The method of claim 10, wherein the thiolated PEG is capped with an alkoxy having 1-20 carbon atoms such as a methoxy at the other end.

12. The method of any of claims 9-11, wherein the modified PEG has a number or weight average molecular weight, preferably, a number average molecular weight, of about 1000-5000 g/mol, such as about 2000 g/mol.

13. The method of any of claims 9-12, wherein the first blocking solution comprises the modified PEG at a concentration about 0.1-10 mM.

14. The method of any of claims 9-13, wherein the blocking step b) further comprises treating the surface-agent-functionalized surface with a second blocking solution comprising a serum protein, wherein the second blocking solution is different from the first blocking solution, and the treatment with the second blocking solution occurs after the treatment with the first blocking solution.

15. The method of claim 14, wherein the serum protein is albumin and/or fibrinogen, preferably, albumin.

16. The method of claim 14, wherein the serum protein is albumin, such as bovine serum albumin.

17. The method of any of claims 14-16, wherein the second blocking solution comprises the serum protein at a concentration of about 0.1% to about 5% (w/v), such as about 1% (w/v).

18. The method of any of claims 9-17, wherein the blocking step b) further comprises treating the surface-agent-functionalized surface with a third blocking solution comprising an antibody, preferably, the antibody is of the IgG isotype, wherein the third blocking solution is different from the first or second blocking solution, and the treatment with the third blocking solution occurs between the treatment with the first and second blocking solutions.

19. The method of claim 18, wherein the antibody is a human antibody, a mouse antibody, and/or a rabbit antibody, for example, the third blocking solution comprises a mixture of human IgG and rabbit IgG.

20. The method of claim 18, wherein the third blocking solution comprises a human IgG antibody at a concentration of about 10-300 μg/mL and a rabbit IgG antibody at a concentration of about 10-300 μg/mL, preferably, the molar ratio of the human IgG to the rabbit IgG ranges from about 0.1:10 to about 10:0.1.

21. The method of any of claims 9-20, wherein the blocking step b) further comprises treating the surface-agent-functionalized surface with a fourth blocking solution comprising a fragment crystallizable (Fc) region of an IgG antibody, such as a human, rabbit, or mouse IgG antibody, preferably, rabbit Fc region, for example, at a concentration of about 0.1 μg/mL to about 10 μg/mL.

22. The method of any of claims 1-8, wherein the blocking step b) comprises treating the surface-agent-functionalized surface with a combined blocking solution comprising (i) a modified polyethylene glycol (PEG), wherein the modified PEG is modified with an amine, carboxyl, or thiol at one end; and (ii) a serum protein.

23. The method of claim 22, wherein the modified PEG is a thiolated PEG, wherein the PEG is modified with a thiol at one end.

24. The method of claim 23, wherein the thiolated PEG is capped with an alkoxy having 1-20 carbon atoms such as a methoxy at the other end.

25. The method of any of claims 22-24, wherein the modified PEG has a number or weight average molecular weight, preferably, a number average molecular weight, of about 1000-5000 g/mol.

26. The method of any of claims 22-25, wherein the combined blocking solution comprises the modified PEG at a concentration about 0.1-10 mM.

27. The method of any of claims 22-26, wherein the serum protein is albumin and/or fibrinogen, preferably, albumin.

28. The method of any of claims 22-26, wherein the serum protein is albumin, such as bovine serum albumin.

29. The method of any of claims 22-28, wherein the combined blocking solution comprises the serum protein at a concentration of about 0.1% to about 5% (w/v), such as about 1% (w/v).

30. The method of any of claims 22-29, wherein the combined blocking solution further comprises an antibody, preferably, the antibody is of the IgG isotype.

31. The method of claim 30, wherein the antibody is a human antibody, a mouse antibody, and/or a rabbit antibody, for example, the combined blocking solution comprises a mixture of human IgG and rabbit IgG.

32. The method of claim 30 or 31, wherein the combined blocking solution comprises a human IgG antibody at a concentration of about 10-300 μg/mL and a rabbit IgG antibody at a concentration of about 10-300 μg/mL, preferably, the molar ratio of the human IgG to the rabbit IgG ranges from about 0.1:10 to about 10:0.1.

33. The method of any of claims 22-32, wherein the combined blocking solution further comprises a fragment crystallizable (Fc) region of an IgG antibody, such as a human, rabbit, or mouse IgG antibody, preferably, rabbit Fc region, for example, at a concentration of about 0.1 μg/mL to about 10 μg/mL.

34. The method of any of claims 1-33, wherein the blocking step b) further comprising treating the surface-agent-functionalized surface with one or more ingredients selected from a buffer (e.g., phosphate buffered saline), a silane (e.g., decafluoro-1,1,2,2,-tetrahydrooctyl trichlorosilane (FOTS)), a surfactant (e.g., egg phosphatidylcholine, palmitoyl-oleoylphosphatidylcholine (POPC), Triton X, Tween 20, etc.), and a thiol (e.g., mercaptopropanol (MPO)).

35. The method of claim 34, wherein the blocking step b) further comprising treating the surface-agent-functionalized surface with the buffer, preferably phosphate-buffered saline (PBS), sodium chloride-sodium phosphate-EDTA, or HEPES (4-(2-hydroxyethyl)-1-piperazineethanesulfonic acid), preferably, the buffer has a pH of about 6-8.

36. The method of claim 34 or 35, wherein the blocking step b) further comprising treating the surface-agent-functionalized surface with the surfactant, such as Tween 20.

37. The method of any of claims 1-36, further comprising treating the surface-agent-functionalized surface with a salt (e.g., sodium chloride) and/or a chelating agent (e.g., ethylenediaminetetraacetic acid (EDTA)).

38. The method of any of claims 1-37, further comprising immobilizing a surface-agent-dependent capture molecule on the surface-agent-functionalized surface prior to the blocking step b), wherein the immobilizing comprises specifically binding the surface-agent-dependent capture molecule to the surface agent directly or indirectly.

39. The method of any of claims 1-38, wherein the substrate is a glass, metal, ceramic, or polymer substrate, preferably a glass substrate.

40. The method of any of claims 1-38, wherein the substrate is a glass substrate suitable for use in a surface plasmon resonance imaging analysis.

41. The substrate having the inert metal surface prepared by the method of any of claims 1-40.

42. A combined blocking solution comprising: (a) a thiolated PEG, wherein the PEG is modified with a thiol at one end; (b) a serum protein; and optionally (c) an antibody, such as a human antibody, a mouse antibody, and/or a rabbit antibody.

43. The combined blocking solution of claim 42, wherein the thiolated PEG is capped with an alkoxy having 1-20 carbon atoms such as a methoxy at the other end.

44. The combined blocking solution of claim 42 or 43, wherein the thiolated PEG has a number or weight average molecular weight, preferably, a number average molecular weight, of about 1000-5000 g/mol.

45. The combined blocking solution of any of claims 42-44, wherein the thiolated PEG is at a concentration about 0.1-10 mM.

46. The combined blocking solution of any of claims 42-45, wherein the serum protein is albumin, such as bovine serum albumin.

47. The combined blocking solution of any of claims 42-46, wherein the serum protein is bovine serum albumin, and the combined blocking solution comprises the bovine serum albumin at a concentration of about 0.1% to about 5% (w/v), such as about 1% (w/v).

48. The combined blocking solution of any of claims 42-47, wherein the combined blocking solution comprises the antibody, such as a human antibody, a mouse antibody, and/or a rabbit antibody, for example, the combined blocking solution comprises a mixture of human IgG and rabbit IgG.

49. The combined blocking solution of claim 48, wherein the combined blocking solution comprises a human IgG antibody at a concentration of about 10-300 μg/mL and a rabbit IgG antibody at a concentration of about 10-300 μg/mL, preferably, the molar ratio of human IgG to the rabbit IgG ranges from about 0.1:10 to about 10:0.1.

50. The combined blocking solution of any of claims 42-49, further comprising a fragment crystallizable (Fc) region of an IgG antibody, such as a human, rabbit, or mouse IgG antibody, preferably, rabbit Fc region, for example, at a concentration of about 0.1 μg/mL to about 10 μg/mL.

51. The combined blocking solution of any of claims 42-50, further comprising a buffer, such as phosphate-buffered saline (PBS), sodium chloride-sodium phosphate-EDTA, or HEPES (4-(2-hydroxyethyl)-1-piperazineethanesulfonic acid), at a pH of about 6-8.

52. A combination of blocking agents comprising (a) a first solution comprising a thiolated PEG, wherein the PEG is modified with a thiol at one end; (b) a second solution comprising a serum protein; and optionally (c) a third solution comprising an antibody, such as a human antibody, a mouse antibody, and/or a rabbit antibody, wherein the first, second, and third solution do not contain the same blocking agent(s).

53. The combination of claim 52, wherein the thiolated PEG is capped with an alkoxy having 1-20 carbon atoms such as a methoxy at the other end.

54. The combination of claim 52 or 53, wherein the thiolated PEG has a number or weight average molecular weight, preferably, a number average molecular weight, of about 1000-5000 g/mol.

55. The combination of any of claims 52-54, wherein the first solution comprises the thiolated PEG at a concentration about 0.1-10 mM.

56. The combination of any of claims 52-55, wherein the serum protein is albumin, such as bovine serum albumin.

57. The combination of any of claims 52-55, wherein the serum protein is bovine serum albumin, and the second solution comprises the bovine serum albumin at a concentration of about 0.1% to about 5% (w/v), such as about 1% (w/v).

58. The combination of any of claims 52-57, comprising the third solution, wherein the third solution comprises a human antibody, a mouse antibody, and/or a rabbit antibody, for example, the third solution comprises a mixture of human IgG and rabbit IgG.

59. The combination of claim 58, wherein the third solution comprises a human IgG antibody at a concentration of about 10-300 μg/mL and a rabbit IgG antibody at a concentration of about 10-300 μg/mL, preferably, the molar ratio of human IgG to the rabbit IgG ranges from about 0.1:10 to about 10:0.1.

60. The combination of any of claims 52-59 further comprising a fourth solution comprising a fragment crystallizable (Fc) region of an IgG antibody, such as a human, rabbit, or mouse IgG antibody, preferably, rabbit Fc region, for example, at a concentration of about 0.1 μg/mL to about 10 μg/mL.

61. The combination of any of claims 52-60, wherein as applicable, the first, second, third, and fourth solution comprise a buffer, such as phosphate-buffered saline (PBS), sodium chloride-sodium phosphate-EDTA, or HEPES (4-(2-hydroxyethyl)-1-piperazineethanesulfonic acid), at a pH of about 6-8.

62. A substrate having an inert metal surface, wherein the inert metal surface is treated with any of the combined blocking solution of claims 42-51 or any of the combination of any of claims 52-61.

63. The substrate of claim 62, wherein the inert metal surface is a gold surface, a silver surface, or a gold/silver alloy surface coated on the substrate.

64. A kit comprising (i) a substrate having an inert metal surface; and (ii) any of the combined blocking solution of claims 42-51 or any of the combination of any of claims 52-61.

65. The kit of claim 64, wherein the inert metal surface is a gold surface, a silver surface, or a gold/silver alloy surface coated on the substrate.

66. The kit of claim 64 or 65, further comprising a surface agent.

67. The kit of any of claims 64-66, wherein the inert metal surface is functionalized with a surface agent.

68. The kit of claim 66 or 67, further comprising a surface-agent-dependent capture molecule, wherein the surface agent is capable of binding to the inert metal surface and specifically binding to the surface-agent-dependent capture molecule, wherein the surface-agent-dependent capture molecule is capable of specifically binding to one or more analytes.

69. The kit of any of claims 64-68, further comprising a surface-agent-independent capture molecule, wherein the surface-agent-independent capture molecule is capable of directly or indirectly binding to the inert surface without binding to a surface agent, and the surface-agent-independent capture molecule is capable of specifically binding to one or more analytes.

70. The kit of any of claims 66-69, wherein the surface agent is a thiolated protein A, wherein the protein A is modified with a thiol.

71. The kit of any of claims 64-70, comprising one or more ingredients selected from a buffer (e.g., phosphate buffered saline), a silane (e.g., decafluoro-1,1,2,2,-tetrahydrooctyl trichlorosilane (FOTS)), a surfactant (e.g., egg phosphatidylcholine, palmitoyl-oleoylphosphatidylcholine (POPC), Triton X, Tween 20, etc.), and a thiol (e.g., mercaptopropanol (MPO)).

72. The kit of any of claims 64-71, comprising a buffer, preferably phosphate-buffered saline (PBS), sodium chloride-sodium phosphate-EDTA, or HEPES (4-(2-hydroxyethyl)-1-piperazineethanesulfonic acid), preferably, the buffer has a pH of about 6-8.

73. The kit of any of claims 64-72, comprising a surfactant, such as Tween 20.

74. The kit of any of claims 64-73, comprising a salt (e.g., sodium chloride) and/or a chelating agent (e.g., ethylenediaminetetraacetic acid (EDTA)).

75. The kit of any of claims 64-74, further comprising one or more components selected from antigens, serum samples, inhibitors that stabilize a pathogen-related DNA or RNA target in the serum samples, detection aptamers, detection antibodies, and nanoenhancers.

76. The kit of any of claims 64-75, wherein the substrate is a glass substrate.

77. The kit of claim 76, wherein the glass substrate is suitable for use in a surface plasmon resonance imaging analysis.

78. A substrate having an inert metal surface, wherein the inert metal surface comprises:
a) a surface-agent-dependent capture molecule, which is immobilized on the inert metal surface through specific binding to a surface agent bound to the inert metal surface; and
b) a plurality of blocking agents, which are bound to the inert metal surface directly or indirectly,
wherein the surface-agent-dependent capture molecule is capable of specifically binding to one or more analytes, wherein the plurality of blocking agents are capable of reducing (preferably substantially reducing) or preventing the inert metal surface from non-specific binding.

79. The substrate of claim 78, wherein the surface agent is a protein, preferably, protein A, protein G, protein L, avidin, or streptavidin.

80. The substrate of claim 79, wherein the surface agent is protein A modified with amine, carboxyl, hydroxyl, and/or thiol.

81. The substrate of claim 79, wherein the surface agent is a thiolated protein A, wherein the protein A is modified with a thiol.

82. The substrate of any one of claims 78-81, wherein the surface-agent-dependent capture molecule is a capture antibody.

83. The substrate of claim 82, wherein the capture antibody is an IgG isotype antibody, and the surface agent is a thiolated protein A.

84. The substrate of any one of claims 78-83, wherein the plurality of blocking agents comprise a modified polyethylene glycol (PEG), wherein the modified PEG is modified with an amine, carboxyl, or thiol at one end.

85. The substrate of claim 84, wherein the modified PEG is a thiolated PEG, wherein the PEG is modified with a thiol at one end.

86. The substrate of claim 85, wherein the thiolated PEG is capped with an alkoxy having 1-20 carbon atoms such as a methoxy at the other end.

87. The substrate of any one of claims 84-86, wherein the modified PEG has a number or weight average molecular weight, preferably, a number average molecular weight, of about 1000-5000 g/mol.

88. The substrate of any one of claims 84-87, wherein the plurality of blocking agents further comprise a serum protein.

89. The substrate of claim 88, wherein the serum protein is albumin and/or fibrinogen, preferably, albumin.

90. The substrate of claim 89, wherein the serum protein is bovine serum albumin.

91. The substrate of any one of claims 84-90, wherein the plurality of blocking agents further comprise an antibody, such as a human antibody, a mouse antibody, and/or a rabbit antibody, preferably, the antibody is of the IgG isotype.

92. The substrate of any one of claims 84-90, wherein the plurality of blocking agents further comprise a mixture of human IgG and rabbit IgG.

93. The substrate of any one of claims 84-92, wherein the plurality of blocking agents further comprise a fragment crystallizable (Fc) region of an IgG antibody, such as a human, rabbit, or mouse IgG antibody, preferably, rabbit Fc region.

94. The substrate of any one of claims 84-93, wherein the plurality of blocking agents further comprise one or more ingredients selected from a buffer (e.g., phosphate buffered saline), a silane (e.g., decafluoro-1,1,2,2,-tetrahydrooctyl trichlorosilane (FOTS)), a surfactant (e.g., egg phosphatidylcholine, palmitoyl-oleoylphosphatidylcholine (POPC), Triton X, Tween 20, etc.), and a thiol (e.g., mercaptopropanol (MPO)).

95. The substrate of any one of claims 78-94, wherein the surface agent is uniformly bound to the inert metal surface.

96. The substrate of any one of claims 74-94, wherein the surface agent is bound to the inert metal surface at a predefined area.

97. The substrate of claim 96, further comprising a surface-agent-independent capture molecule, directly or indirectly bound to the inert metal surface without binding to the surface agent, for example, the surface-agent-independent capture molecule is a capture aptamer.

98. The substrate of any one of claims 74-97, wherein the inert metal surface is a gold surface, a silver surface, or a gold/silver alloy surface coated on the substrate.

99. The substrate of any one of claims 74-98, which is a glass, metal, ceramic, or polymer substrate, preferably a glass substrate.

100. The substrate of any one of claims 74-99, which is a glass substrate suitable for use in a surface plasmon resonance imaging analysis.

101. A method of analyzing a sample, comprising (a) providing the substrate of any one of claims 41, 62, 63, and 78-100, wherein the substrate comprises at least one capture molecule on the inert metal surface that is capable of specifically binding to an analyte; (b) incubating the sample with the substrate under a condition suitable for the at least one capture molecule to specifically bind to the analyte; and (c) determining whether the sample specifically binds the substrate, thereby determining whether the analyte is present in the sample.

102. The method of claim 101, wherein the determining step c) comprises comparing surface plasmon resonance reflectivity of the substrate incubated with the sample or a control.


SEQUENCE LISTING (43 sequences)

SEQ ID NO: 1 - protein, 250 aa, unidentified
AAQHDEAQQNAFYQVLNMPNLNADQRNGFIQSLKDDPSQSANVLGEAKKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQQNAFYEILHLPNLTEEQRNGFIQSLKDDPSVSKEILAEAKKLNDAQAP

SEQ ID NO: 2 - protein, 55 aa, unidentified
AQHDEAQQNAFYQVLNMPNLNADQRNGFIQSLKDDPSQSANVLGEAQKLNDSQAP

SEQ ID NO: 3 - protein, 85 aa, unidentified
ADAQQNNFNKDQQSAFYEILNMPNLNEAQRNGFIQSLKDDPSQSTNVLGEAKKLNESQAPSLKDDPSQSTNVLGEAKKLNESQAP

SEQ ID NO: 4 - protein, 96 aa, unidentified
ADNNFNKEQQNAFYEILNMPNLNEEQRNGFIQSLKDDPSQSANLLSEAKKLNESQAPMPNLNEEQRNGFIQSLKDDPSQSANLLSEAKKLNESQAP

SEQ ID NO: 5 - protein, 57 aa, unidentified
ADNKFNKEQQNAFYEILHLPNLNEEQRNGFIQSLKDDPSQSANLLAEAKKLNDAQAP

SEQ ID NO: 6 - protein, 57 aa, unidentified
ADNKFNKEQQNAFYEILHLPNLTEEQRNGFIQSLKDDPSVSKEILAEAKKLNDAQAP

SEQ ID NO: 7 - protein, 73 aa, unidentified
VDNKFNKEQQNAFYEILHLPNLNEEQRNAFIQSLKDDPSQSANLLAEAKKLNDAQAPANLLAEAKKLNDAQAP

SEQ ID NO: 8 - protein, 33 aa, unidentified
FNMQQQRRFYEALHDPNLNEEQRNAKIKSIRDD

SEQ ID NO: 9 - protein, 183 aa, unidentified
GETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTE

SEQ ID NO: 10 - protein, 44 aa, unidentified
GETTTEAVDAATAEKVFKQYANDNGVDGEWTYDDATKTFTVTEK

SEQ ID NO: 11 - protein, 62 aa, unidentified
ELTPAVTTYKLVINGKTLKGETTTEAVDAATAEKVFKQYANDNGVDGEWTYDDATKTFTVTE

SEQ ID NO: 12 - protein, 62 aa, unidentified
ELTPAVTTYKLVINGKTLKGETTTKAVDAETAEKAFKQYANDNGVDGVWTYDDATKTFTVTE

SEQ ID NO: 13 - protein, 292 aa, unidentified
KEETPETPETDSEEEVTIKANLIFANGSTQTAEFKGTFEKATSEAYAYADTLKKDNGEYTVDVADKGYTLNIKFAGKEKTPEEPKEEVTIKANLIYADGKTQTAEFKGTFEEATAEAYRYADALKKDNGEYTVDVADKGYTLNIKFAGKEKTPEEPKEEVTIKANLIYADGKTQTAEFKGTFEEATAEAYRYADLLAKENGKYTVDVADKGYTLNIKFAGKEKTPEEPKEEVTIKANLIYADGKTQTAEFKGTFAEATAEAYRYADLLAKENGKYTADLEDGGYTINIRFAG

SEQ ID NO: 14 - protein, 76 aa, unidentified
KEETPETPETDSEEEVTIKANLIFANGSTQTAEFKGTFEKATSEAYAYADTLKKDNGEYTVDVADKGYTLNIKFAG

SEQ ID NO: 15 - protein, 72 aa, unidentified
KEKTPEEPKEEVTIKANLIYADGKTQTAEFKGTFEEATAEAYRYADALKKDNGEYTVDVADKGYTLNIKFAG

SEQ ID NO: 16 - protein, 72 aa, unidentified
KEKTPEEPKEEVTIKANLIYADGKTQTAEFKGTFEEATAEAYRYADLLAKENGKYTVDVADKGYTLNIKFAG

SEQ ID NO: 17 - protein, 72 aa, unidentified
KEKTPEEPKEEVTIKANLIYADGKTQTAEFKGTFAEATAEAYRYADLLAKENGKYTADLEDGGYTINIRFAG

SEQ ID NO: 18 - protein, 6 aa, unidentified
WQRHGI

SEQ ID NO: 19 - protein, 6 aa, unidentified
MWRGWQ

SEQ ID NO: 20 - protein, 6 aa, unidentified
RHLGWF

SEQ ID NO: 21 - protein, 6 aa, unidentified
GWLHQR

SEQ ID NO: 22 - protein, 12 aa, unidentified
EPIHRSTLTALL

SEQ ID NO: 23 - protein, 6 aa, unidentified
HWRGWV

SEQ ID NO: 24 - protein, 6 aa, unidentified
HYFKFD

SEQ ID NO: 25 - protein, 6 aa, unidentified
HFRRHL

SEQ ID NO: 26 - protein, 6 aa, unidentified
HWCGWV

SEQ ID NO: 27 - protein, 6 aa, unidentified
RWHYFK

SEQ ID NO: 28 - protein, 6 aa, unidentified
WFRHYK

SEQ ID NO: 29 - protein, 8 aa, unidentified
NKFRGKYK

SEQ ID NO: 30 - protein, 8 aa, unidentified
NARKFYKG

SEQ ID NO: 31 - protein, 8 aa, unidentified
FYWHCLDE

SEQ ID NO: 32 - protein, 8 aa, unidentified
FYCHWALE

SEQ ID NO: 33 - protein, 8 aa, unidentified
FYCHTIDE

SEQ ID NO: 34 - protein, 4 aa, unidentified
RRGW

SEQ ID NO: 35 - protein, 7 aa, unidentified
KHRFNKD

SEQ ID NO: 36 - protein, 6 aa, unidentified
RWHYFK

SEQ ID NO: 37 - protein, 13 aa, unidentified
DCAWHLGELVWCT

SEQ ID NO: 38 - protein, 10 aa, unidentified
TWKTSRISIF

SEQ ID NO: 39 - protein, 159 aa, unidentified
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 40 - protein, 126 aa, unidentified
EAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 41 - protein, 128 aa, unidentified
ARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 42 - protein, 141 aa, unidentified
MSDVQSSLTGTWYNELNSKMELTANKDGTLTGKYLSKVGDVYVPYPLSGRYNLQPPAGQGVALGWAVSWENSKIHSATTWSGQFFSESSPVILTQWLLSSSTARGDVWESTLVGNDSFTKTAPTEQQIAHAQLHCRAPRLK

SEQ ID NO: 43 - protein, 141 aa, unidentified
MSDVQSSLTGTWYNELNSKMELTANKDGTLTGKYLAKVGDVYVPYPLSGRYNLQPPAGQGVALGWAVSWENSKIHSATTWSGQFFSESSPVILTQWLLSSSTARGDVWESTLVGNDSFTKTAPTEQQIAHAQLHCRAPRLK